(19) United States
(12) Patent Application Publication
(10) Pub. No.: US 2003/0224411 A1
(43) Pub. Date: Dec. 4, 2003

(54) Genes that are up- or down-regulated during differentiation of human embryonic stem cells

(76) Inventors: Lawrence W. Stanton, Singapore (SG); Ralph Brandenberger, Menlo Park, CA (US); Joseph D. Gold, San Francisco, CA (US); John M. Irving, San Mateo, CA (US); Ramkumar Mandalam, Union City, CA (US); Michael Mok, Palo Alto, CA (US); Dawne Shelton, Salt Lake City, UT (US)

Correspondence Address: GERON CORPORATION, 230 CONSTITUTION DRIVE, MENLO PARK, CA, 94025

(21) Appl. No.: 10/388,578

(22) Filed: Mar. 13, 2003

(51) Int. Cl. C12Q 1/68; G01N 33/53; G01N 33/567; C12N 5/08

(52) U.S. Cl. 435/6; 435/7.2; 435/366

(57) ABSTRACT

Genes that are up- or down-regulated during differentiation provide important leverage by which to characterize and manipulate early-stage pluripotent stem cells. Over 35,000 unique transcripts have been amplified and sequenced from undifferentiated human embryonic stem cells, and three types of differentiated progeny. Statistical analysis of the assembled transcripts identified genes that alter expression levels as differentiation proceeds. The expression profile provides a marker system that has been used to identify particular culture components for maintaining the undifferentiated phenotype. The gene products can also be used to promote differentiation; to assess other relatively undifferentiated cells (such as cancer cells); to control gene expression; or to separate cells having desirable characteristics. Manipulation of particular genes can be used to forestall or focus the differentiation process, en route to producing a specialized homogenous cell population suitable for human therapy.

### TECHNICAL FIELD

This invention relates generally to the field of cell biology of stem cells. More specifically, it relates to phenotypic markers that can be used to characterize, qualify, and control differentiation of pluripotent cells, and to evaluate clinical conditions associated with marker expression.

### BACKGROUND

A promising development in the field of regenerative medicine has been the isolation and propagation of human stem cells from the early embryo. These cells have two very special properties: First, unlike other normal mammalian cell types, they can be propagated in culture almost indefinitely, providing a virtually unlimited supply. Second, they can be used to generate a variety of tissue types of interest as a source of replacement cells and tissues for use in therapy.

Thomson et al. (Science 282:114, 1998; U.S. Pat. No. 6,200,806) were the first to successfully isolate and propagate embryonic stem cells from human blastocysts. Gearhart and coworkers derived human embryonic germ cell lines from fetal gonadal tissue (Shamblott et al., Proc. Natl. Acad. Sci. USA 95:13726, 1998;U.S. Pat. No. 6,090,622).

International Patent Publication WO 99/20741 (Geron Corp.) describes methods and materials for the growth of primate-derived primordial stem cells. International Patent Publication WO 01/51616 (Geron Corp.) provides techniques for growth and differentiation of human pluripotent stem cells. An article by Xu et al. (Nature Biotechnology 19:971, 2001) describes feeder-free growth of undifferentiated human embryonic stem cells. Lebkowski et al. (Cancer J. 7 Suppl. 2:S83, 2001) discuss the culture, differentiation, and genetic modification of human embryonic stem cell for regenerative medicine applications. These publications report exemplary culture methods for propagating human embryonic stem cells in an undifferentiated state, and their use in preparing cells for human therapy.

Markers for identifying undifferentiated pluripotent stem cells include SSEA-4, Tra-1-60, and Tra-1-81 (Thomson et al. and Gearhart et al., supra). They also express human telomerase reverse transcriptase, and the POU transcription factor Oct 3/4 (WO 01/51616; Amit et al., Dev. Biol. 227:271, 2000; Xu et al., supra).

Loring et al. (Restor. Neurol. Neurosci. 18:81, 2001) review gene expression profiles of embryonic stem cells and ES-derived neurons. Pesce et al. (Bioessays 20:722, 1998) comment on the potential role of transcription factor Oct-4 in the totipotent germ-line cycle of mice. Gajovic et al. (Exp. Cell Res. 242:138, 1998) report that genes expressed after retinoic acid-mediated differentiation of embryoid bodies are likely to be expressed during embryo development. Zur Nieden et al. (Toxicol. in Vitro 15:455, 2001) propose certain molecular markers for embryonic stem cells. Henderson et al. (Stem Cells 20:329, 2002) report that pre-implantation human embryos and ES cells have comparable expression of SSEAs. Tanaka et al. (Genome Res. 12:1921, 2002) profile gene expression in mouse ES cells to identify candidate genes associated with pluripotency and lineage specificity. Draper et al. (J. Anat. 299:249, 2002) review change of surface antigens of human embryonic stem cells upon differentiation in culture.

Kelly et al. (Mol Reprod. Dev. 56:113, 2000) report DNA microarray analyses of genes regulated during the differentiation of embryonic stem cells. Woltjen et al. (Nucl. Acids Res. 28:E41, 2000) report retro-recombination screening of a mouse embryonic stem cell genomic library. Monk et al. (Oncogene 20:8085, 2001) list human embryonic genes re-expressed in cancer cells. Tanaka et al. (Genome Res. 12:1921, 2002) discuss gene expression profiling of embryo-derived stem cells, and candidate genes putatively associated with pluripotency and lineage specificity. Monk et al. report developmental genes identified by differential display (Reprod. Fertil. Dev. 13:51, 2001). Natale et al. (Reprod. 122:687, 2001) characterize bovine blastocyst gene expression patterns by differential display RT-PCR.

Fan et al. (Dev. Biol. 210:481,1999) propose that forced expression of the homeobox-containing gene Pem blocks differentiation of embryonic stem cells. Abdel-Rahman et al. (Hum. Reprod. 10:2787, 1995) report the effect of expressing transcription regulating genes in human preimplantation embryos. Jackson et al. (J. Biol. Chem. 277:38683, 2002) describe the cloning and characterization of Ehox, a homeobox gene that reportedly plays a role in ES cell differentiation.

The following disclosure provides new markers and marker combinations that are effective means to identify, characterize, qualify, and control differentiation of pluripotent cells.

### SUMMARY OF THE INVENTION

This invention identifies a number of genes that are up- or down-regulated during the course of differentiation of early-stage pluripotent stem cells obtained from primates, exemplified by human embryonic stem cells. As a consequence, the genes are differentially expressed in undifferentiated versus differentiated cells. This property confers special benefit on these genes for identification, characterization, culturing, differentiation, and manipulation of stem cells and their progeny, and other cells that express the same markers.

One aspect of this invention is a system for assessing a culture of undifferentiated primate pluripotent stem (pPS) cells or their progeny, in which expression of one or more of the identified markers listed in the disclosure is detected or measured. The level of expression can be measured in isolation or compared with any suitable standard, such as undifferentiated pPS cells maintained under specified conditions, progeny at a certain stage of differentiation, or stable end-stage differentiated cells, such as may be obtained from the ATCC. Depending on whether the marker(s) are up- or down-regulated during differentiation, presence of the markers is correlated with the presence or proportion of undifferentiated or differentiated cells in the population.

An exemplary (non-limiting) combination suitable for qualifying cultures of undifferentiated pPS cells is a marker selected from the list of Cripto, gastrin-releasing peptide (GRP) receptor, and podocalyxin-like protein, in combination with either hTERT and/or Oct 3/4 (POU domain, class 5 transcription factor), or a second marker from the list. Additional markers can also be measured as desired. Markers can be detected at the mRNA level by PCR amplification, at the protein or enzyme product level by antibody assay, or by any suitable technique.

The marker system of this invention can be used for quantifying the proportion of undifferentiated pPS cells or differentiated cells in the culture; for assessing the ability of a culture system or component thereof (such as a soluble factor, culture medium, or feeder cell) to maintain pPS cells in an undifferentiated state; for assessing the ability of a culture system or component thereof to cause differentiation of pPS cells into a culture of lineage-restricted precursor cells or terminally differentiated cells; or for any other worthwhile purpose. This invention includes kits and the use of specific reagents in order to measure the expression of the markers whenever appropriate.

This invention also provides a system assessing the growth characteristics of a cell population by detecting or measuring expression of one or more of the differentially expressed marker genes identified in this disclosure. This can be applied not only to various types of pPS cells and progenitor cells in various stages of differentiation, but also to clinical samples from a disease condition associated with abnormal cell growth. Renewed expression of markers of a relatively undifferentiated phenotype may be diagnostic of disease conditions such as cancer, and can serve as a means by which to target therapeutic agents to the disease site.

The marker system can also be used to regulate gene expression. Transcriptional control elements for the markers will cause an operatively linked encoding region to be expressed preferentially in undifferentiated or differentiated cells. For example, the encoding sequence can be a reporter gene (such as a gene that causes the cells to emit fluorescence), a positive selection marker (such as a drug resistance gene), or a negative selection marker. Vector constructs comprising recombinant elements linked in this fashion can be used to positively select or deplete undifferentiated, differentiated, or cancerous cells from a mixed population or in vivo, depending on the nature of the effector gene and whether transcription is up- or down-regulated during differentiation. They can also be used to monitor culture conditions of pPS cells, differentiation conditions, or for drug screening.

The marker system of this invention can also be used to sort differentiated cells from less differentiated cells. The marker can be used directly for cell separation by adsorption using an antibody or lectin, or by fluorescence activated cell sorting. Alternatively, these separation techniques can be effected using a transcription promoter from the marker gene in a promoter-reporter construct.

The marker system of this invention can be used to map differentiation pathways or influence differentiation. Markers suited for this purpose may act as transcription regulators, or encode products that enhance cell interaction in some fashion. pPS cells or their differentiated progeny are genetically altered to increase expression of one or more of the identified genes using a transgene, or to decrease expression, for example, using an antisense or siRNA construct. Alternatively, gene products involved in cell interaction or signaling can be added directly to the culture medium. The effect of this can be to help maintain the transfected cell in the undifferentiated state, promote differentiation in general, or direct differentiation down a particular pathway.

Another aspect of the invention are methods for identifying these and other genes that are up- or down-regulated upon differentiation of any cell type. The methods involve comparing expression libraries obtained from the cells before and after differentiation, by sequencing transcripts in each of the libraries, and identifying genes that have statistically significant differences in the relative number of transcripts (as a percentage of transcripts in each library) at a confidence level of 67%, 95%, or 98%. The method can be enhanced by creating assemblies in which different sequences are counted for the same transcript if they are known to correspond to a single transcript according to previously compiled data.

Amongst the differentially expressed markers identified in this disclosure are 39 nucleotide sequences which are not present in their entirety in the UniGene database. These are listed in this disclosure as SEQ. ID NOs:101 to 139. This invention includes novel nucleic acids consisting of or containing any of these sequences or the complementary sequences, and novel fragments thereof. This invention also includes novel polypeptides encoded in these sequences (made either by expressing the nucleic acid or by peptide synthesis), antibodies specific for the polypeptides (made by conventional techniques or through a commercial service), and use of these nucleic acids, peptides, and antibodies for any industrial application.

Also embodied in this invention are culture conditions and other cell manipulations identified using the marker system of this invention that are suitable for maintaining or proliferating pPS cells without allowing differentiation, or causing them to differentiate in a certain fashion. Culture conditions tested and validated according to this invention are illustrated in the example section.

Other embodiments of the invention will be apparent from the description that follows.

### DRAWINGS

FIG. 1 shows the profile of genes preferentially expressed in undifferentiated pluripotent stem cells, upon preliminary differentiation of the cells by culturing in retinoic acid or DMSO. Level of gene expression at the mRNA level was measured by real-time PCR assay. Any of the genes showing substantial down-regulation upon differentiation can be used to characterize the undifferentiated cell population, and culture methods suitable for maintaining them in an undifferentiated state.

FIG. 2 shows the level of expression of five genes in hES cells, compared with fully differentiated cells. This five-marker panel provides robust qualification of the undifferentiated phenotype.

FIG. 3 show results of an experiment in which hES cells of the H1 line were maintained for multiple passages in different media. Medium conditioned with feeder cells provides factors effective to allow hES cells to proliferate in culture without differentiating. However, culturing in unconditioned medium leads to decreased percentage of cells expressing CD9, and the classic hES cell marker SSEA-4.

FIG. 4 illustrates the sensitivity of hTERT, Oct 3/4, Cripto, GRP receptor, and podocalyxin-like protein (measured by real-time PCR) as a means of determining the degree of differentiation of the cells. After multiple passages in unconditioned medium, all five markers show expression that has been downregulated by 10 to 10⁴-fold.

FIG. 5 shows results of an experiment in which the hES cell line H1 was grown on different feeder cell lines: mEF=mouse embryonic fibroblasts; hMSC=human mesenchymal stem cells; UtSMC =uterine smooth muscle cells; WI-38=human lung fibroblasts. As monitored using Cripto, the hMSC is suitable for use as feeder cells to promote hES cell proliferation without differentiation.

FIG. 6 shows results of an experiment in which different media were tested for their ability to promote growth of hES cells without proliferation. The test media were not preconditioned, but supplemented with 8-40 ng/mL bFGF, with or without stem cell factor, Flt3 ligand, or LIF. Effective combinations of factors (Conditions 4 to 8) were identified by following the undifferentiated phenotype using the markers of this invention. Alterations in expression profiles were temporary and reversible, showing that the cells are still undifferentiated.

### DETAILED DESCRIPTION

The propensity of pluripotent stem cells to differentiate spontaneously has made it challenging for investigators to work with these cells. Consistent cultures of undifferentiated stem cells are required to compare results obtained from multiple experiments performed within or between laboratories. Unfortunately, morphological characterization is subjective and especially difficult for cultures that often contain 10-20% differentiated cells. Nevertheless, having a set of standardized criteria will be important in qualifying these cells for use in clinical therapy.

The marker system identified in this disclosure provides the basis for establishing these standards. 148,453 different transcripts were amplified and sequenced from undifferentiated human embryonic stem cells, and three types of progeny. As a result of this sequencing effort, 532 genes were identified having substantially higher EST counts in undifferentiated cells, and 142 genes were identified having substantially higher EST counts after differentiation. Other differentially expressed genes were identified by microarray analysis of undifferentiated cells, compared with cells at the beginning of the differentiation process.

The system provided by this invention can be used not only to qualify populations of undifferentiated cells, but in other powerful ways of maintaining and manipulating cells described later in this disclosure. Culture systems have been identified and protocols have been developed to expand cultures of undifferentiated cells and produce commercially viable quantities of cells for use in research, drug screening, and regenerative medicine.

### Definitions

“Pluripotent Stem cells” (pPS cells) are pluripotent cells that have the characteristic of being capable under appropriate conditions of producing progeny of several different cell types that are derivatives of all of the three germinal layers (endoderm, mesoderm, and ectoderm), according to a standard art-accepted test, such as the ability to form a teratoma in 8-12 week old SCID mice. The term includes both established lines of stem cells of various kinds, and cells obtained from primary tissue that are pluripotent in the manner described. For the purposes of this disclosure, the pPS cells are not embryonal carcinoma (EC) cells, and are not derived from a malignant source. It is desirable (but not always necessary) that the cells be euploid. Exemplary pPS cells are obtained from embryonic or fetal tissue at any time after fertilization.

“Human Embryonic Stem cells” (hES cells) are pluripotent stem cells derived from a human embryo in the blastocyst stage, or human pluripotent cells produced by artificial means (such as by nuclear transfer) that have equivalent characteristics. Exemplary derivation procedures and features are provided in a later section.

hES cell cultures are described as “undifferentiated” when a substantial proportion (at least 20%, and possibly over 50% or 80%) of stem cells and their derivatives in the population display morphological characteristics of undifferentiated cells, distinguishing them from differentiated cells of embryo or adult origin. It is understood that colonies of undifferentiated cells within the population will often be surrounded by neighboring cells that are differentiated. It is also understood that the proportion of cells displaying the undifferentiated phenotype will fluctuate as the cells proliferate and are passaged from one culture to another. Cells are recognized as proliferating in an undifferentiated state when they go through at least 4 passages and/or 8 population doublings while retaining at least about 50%, or the same proportion of cells bearing characteristic markers or morphological characteristics of undifferentiated cells.

A “differentiated cell” is a cell that has progressed down a developmental pathway, and includes lineage-committed progenitor cells and terminally differentiated cells.

“Feeder cells” or “feeders” are terms used to describe cells of one type that are co-cultured with cells of another type, to provide an environment in which the cells of the second type can grow. hES cell populations are said to be “essentially free” of feeder cells if the cells have been grown through at least one round after splitting in which fresh feeder cells are not added to support the growth of pPS cells.

The term “embryoid bodies” refers to aggregates of differentiated and undifferentiated cells that appear when pPS cells overgrow in monolayer cultures, or are maintained in suspension cultures. Embryoid bodies are a mixture of different cell types, typically from several germ layers, distinguishable by morphological criteria and cell markers detectable by immunocytochemistry.

A cell “marker” is any phenotypic feature of a cell that can be used to characterize it or discriminate it from other cell types. A marker of this invention may be a protein (including secreted, cell surface, or internal proteins; either synthesized or taken up by the cell); a nucleic acid (such as an mRNA, or enzymatically active nucleic acid molecule) or a polysaccharide. Included are determinants of any such cell components that are detectable by antibody, lectin, probe or nucleic acid amplification reaction that are specific for the cell type of interest. The markers can also be identified by a biochemical or enzyme assay that depend on the function of the gene product. Associated with each marker is the gene that encodes the transcript, and the events that lead to marker expression.

The terms “polynucleotide” and “nucleic acid” refer to a polymeric form of nucleotides of any length. Included are genes and gene fragments, mRNA, cDNA, plasmids, viral and non-viral vectors and particles, nucleic acid probes, amplification primers, and their chemical equivalents. As used in this disclosure, the term polynucleotide refers interchangeably to double- and single-stranded molecules. Unless otherwise specified, any embodiment of the invention that is a polynucleotide encompasses both a double-stranded form, and each of the two complementary single-stranded forms known or predicted to make up the double-stranded form.

A cell is said to be “genetically altered” or “transtected” when a polynucleotide has been transferred into the cell by any suitable means of artificial manipulation, or where the cell is a progeny of the originally altered cell that has inherited the polynucleotide.

A “control element” or “control sequence” is a nucleotide sequence involved in an interaction of molecules that contributes to the functional regulation of a polynucleotide, including replication, duplication, transcription, splicing, translation, or degradation of the polynucleotide. “Operatively linked” refers to an operative relationship between genetic elements, in which the function of one element influences the function of another element. For example, an expressible encoding sequence may be operatively linked to a promoter that drives gene transcription.

The term “antibody” as used in this disclosure refers to both polyclonal and monoclonal antibody. The ambit of the term deliberately encompasses not only intact immunoglobulin molecules, but also such fragments and derivatives of immunoglobulin molecules that retain a desired binding specificity.

### General Techniques

Methods in molecular genetics and genetic engineering are described generally in the current editions of Molecular Cloning: A Laboratory Manual, (Sambrook et al.); Oligonucleotide Synthesis (M. J. Gait, ed.); Animal Cell Culture (R.l. Freshney, ed.); Gene Transfer Vectors for Mammalian Cells (Miller & Calos, eds.); Current Protocols in Molecular Biology and Short Protocols in Molecular Biology, 3rd Edition (F. M. Ausubel et al., eds.); and Recombinant DNA Methodology (R. Wu ed., Academic Press). Antibody production is described in Basic Methods in Antibody Production and Characterization (Howard & Bethell eds., CRC Press, 2000).

A survey of relevant techniques is provided in such standard texts as DNA Sequencing (A. E. Barron, John Wiley, 2002), and DNA Microarrays and Gene Expression (P. Baldi et al., Cambridge U. Press, 2002). For a description of the molecular biology of cancer, the reader is referred to Principles of Molecular Oncology (M. H. Bronchud et al. eds., Humana Press, 2000); The Biological Basis of Cancer (R. G. McKinnel et al. eds., Cambridge University Press, 1998); and Molecular Genetics of Cancer (J. K. Cowell ed., Bios Scientific Publishers, 1999).

Sources of Stem Cells

This invention is based on observations made with established lines of hES cells. The markers are suitable for identifying, characterizing, and manipulating related types of undifferentiated pluripotent cells. They are also suitable for use with pluripotent cells obtained from primary embryonic tissue, without first establishing an undifferentiated cell line. It is contemplated that the markers described in this application will in general be useful for other types of pluripotent cells, including embryonic germ cells (U.S. Pat. Nos. 6,090,622 and 6,251,671), and ES and EG cells from other mammalian species, such as non-human primates.

Embryonic Stem Cells

Embryonic stem cells can be isolated from blastocysts of members of primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, outlined in WO 01/51610 (Bresagen).

hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (˜200 U/mL; Gibco) or by selection of individual colonies by micropipette. Clump sizes of about 50 to 100 cells are optimal.

Propagation of pPS Cells in an Undifferentiated State

pPS cells can be propagated continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (US 20020076747 A1, Life Technologies Inc.), 1% non-essential amino acids, 1 mM L-glutamine, and 0.1 mM β-mercaptoethanol. Just before use, human bFGF is added to 4 ng/mL (WO 99/20741, Geron Corp.).

Traditionally, ES cells are cultured on a layer of feeder cells, typically fibroblasts derived from embryonic or fetal tissue. Embryos are harvested from a CF1 mouse at 13 days of pregnancy, transferred to 2 mL trypsin/EDTA, finely minced, and incubated 5 min at 37° C. 10% FBS is added, debris is allowed to settle, and the cells are propagated in 90% DMEM, 10% FBS, and 2 mM glutamine. To prepare a feeder cell layer, cells are irradiated to inhibit proliferation but permit synthesis of factors that support ES cells (˜4000 rads γ-irradiation). Culture plates are coated with 0.5% gelatin overnight, plated with 375,000 irradiated mEFs per well, and used 5 h to 4 days after plating. The medium is replaced with fresh hES medium just before seeding pPS cells.

Scientists at Geron have discovered that pPS cells can be maintained in an undifferentiated state even without feeder cells. The environment for feeder-free cultures includes a suitable culture substrate, particularly an extracellular matrix such as Matrigel® or laminin. The pPS cells are plated at >15,000 cells cm⁻² (optimally 90,000 cm⁻² to 170,000 cm⁻²). Typically, enzymatic digestion is halted before cells become completely dispersed (say, ˜5 min with collagenase IV). Clumps of ˜10 to 2,000 cells are then plated directly onto the substrate without further dispersal. Alternatively, the cells can be harvested without enzymes before the plate reaches confluence by incubating ˜5 min in a solution of 0.5 mM EDTA in PBS. After washing from the culture vessel, the cells are plated into a new culture without further dispersal. In a further illustration, confluent human embryonic stem cells cultured in the absence of feeders are removed from the plates by incubating with a solution of 0.05% (wt/vol) trypsin (Gibco) and 0.053 mM EDTA for 5-15 min at 37° C. The remaining cells in the plate are removed and the cells are triturated into a suspension comprising single cells and small clusters, and then plated at densities of 50,000-200,000 cells cm⁻² to promote survival and limit differentiation.

Feeder-free cultures are supported by a nutrient medium containing factors that support proliferation of the cells without differentiation. Such factors may be introduced into the medium by culturing the medium with cells secreting such factors, such as irradiated (˜4,000 rad) primary mouse embryonic fibroblasts, telomerized mouse fibroblasts, or fibroblast-like cells derived from pPS cells. Medium can be conditioned by plating the feeders at a density of ˜5-6×10⁴ cm⁻² in a serum free medium such as KO DMEM supplemented with 20% serum replacement and 4 ng/mL bFGF. Medium that has been conditioned for 1-2 days is supplemented with further bFGF, and used to support pPS cell culture for 1-2 days. Alternatively or in addition, other factors can be added that help support proliferation without differentiation, such as ligands for the FGF-2 or FGF-4 receptor, ligands for c-kit (such as stem cell factor), ligands for receptors associated with gp 130, insulin, transferrin, lipids, cholesterol, nucleosides, pyruvate, and a reducing agent such as β-mercaptoethanol. Aspects of the feeder-free culture method are further discussed in International Patent Publications WO 99/20741, WO 01/51616; Xu et al., Nat. Biotechnol. 19:971, 2001; and PCT application PCT/US02/28200. Exemplary culture conditions tested and validated using the marker system of this invention are provided below in Example 6.

Under the microscope, ES cells appear with high nuclear/cytoplasmic ratios, prominent nucleoli, and compact colony formation with poorly discernable cell junctions. Conventional markers for hES cells are stage-specific embryonic antigen (SSEA) 3 and 4, and markers detectable using antibodies Tra-1-60 and Tra-1-81 (Thomson et al., Science 282:1145, 1998). Differentiation of pPS cells in vitro results in the loss of SSEA-4, Tra-1-60, and Tra-1-81 expression, and increased expression of SSEA-1.

### Markers of Undifferentiated pPS Cells and Their Differentiated Progeny

The tables and description provided later in this disclosure provide markers that distinguish undifferentiated pPS cells from their differentiated progeny.

Expression libraries were made from ES cells (WO 01/51616), embryoid bodies (WO 01/51616), and cells differentiated towards the hepatocyte (WO 01/81549) or neural cell (WO 01/88104) lineage. mRNA was reverse transcribed and amplified, producing expressed sequence tags (ESTs) occurring in frequency proportional to the level of expression in the cell type being analyzed. The ESTs were subjected to automatic sequencing, and counted according to the corresponding unique (non-redundant) transcript. A total of 148,453 non-redundant transcripts were represented in each of the 4 libraries. Genes were then identified as having a differential expression pattern if the number of EST counts of the transcript was statistically different between the libraries being compared.

In a parallel set of experiments, mRNA from each of the cell types was analyzed for binding to a broad-specificity EST-based microarray, performed according to the method described in WO 01/51616. Genes were identified as having a differential expression pattern if they showed a comparatively different signal on the microarray.

Significant expression differences determined by EST sequencing, microarray analysis, or other observations were confirmed by real-time PCR analysis. The mRNA was amplified by PCR using specific forward and reverse primers designed from the GenBank sequence, and the amplification product was detected using labeled sequence-specific probes. The number of amplification cycles required to reach a threshold amount was then compared between different libraries.

Distinguishing markers fall into several categories. Those of particular interest include the following:

Markers characteristically expressed at a higher level in undifferentiated pPS cells than any of the differentiated cells, indicating down-regulation during differentiation. The gene products may be involved in maintaining the undifferentiated phenotype.

Markers characteristically expressed at a higher level in the three differentiated cell types than in the undifferentiated cells, indicating up-regulation during differentiation. The gene products may be involved in the general differentiation process.

Markers characteristically expressed at a higher level in one of the differentiated cell types. The encoded genes may be involved in differentiation down restricted lineages.

Markers can also be classified according to the function of the gene product or its location in the cell. Where not already indicated, protein gene products can be predicted by referencing public information according to the GenBank accession number, or by translating the open reading frame after the translation start signal though the genetic code. Features of the markers listed can be determined by the descriptors give in the tables below, or by using the accession number or sequence data to reference public information. Marker groups of particular interest include the following:

Secreted proteins—of interest, for example, because they can be detected by immunoassay of the culture supernatant, and may transmit signals to neighboring cells. Secreted proteins typically have an N-terminal signal peptides, and may have glycosylation sites.

Surface membrane proteins—of interest, for example, because they can be used for cell-surface labeling and affinity separation, or because they act as receptors for signal transduction. They may have glycosylation sites and a membrane spanning region. A Markov model for predicting transmembrane protein topology is described by Krogh et al., J. Mol Biol. 305:567, 2001.

Enzymes with relevant function. For example, enzymes involved in protein synthesis and cleavage or in apoptosis may influence differentiation. Glycosyltransferases decorate the cell membrane with distinguishing carbohydrate epitopes that may play a role in cellular adhesion or localization.

Transcription regulatory factors—of interest for their potential to influence differentiation, as explained later in this disclosure. These factors sometimes have zinc fingers or other identifiable topological features involved in the binding or metabolism of nucleic acids.

Through the course of this work, the key signaling pathways Wnt, Sonic hedgehog (Shh), and Notch emerged as regulators of growth of pPS cells. Interestingly, these pathways have also been shown to play a role in the growth of tumor cells of various kinds, and in embryonic development of lower species.

Now that genes have been identified that are up-regulated or down-regulated upon differentiation, a number of commercial applications of these markers will be apparent to the skilled reader. The sections that follow provide non-limiting illustrations of how some of these embodiments can be implemented.

### Use of Cell Markers to Characterize DPS Cells and Their Differentiated Progeny

The markers provided in this disclosure can be used as a means to identify both undifferentiated and differentiated cells—either a population as a whole, or as individual cells within a population. This can be used to evaluate the expansion or maintenance of pre-existing cell populations, or to characterize the pluripotent nature (or lineage commitment) of newly obtained populations.

Expression of single markers in a test cell will provide evidence of undifferentiated or differentiated phenotype, according to the expression pattern listed later in this disclosure. A plurality of markers (such as any 2, 3, 4, 5, 6, 8, 10, 12, 15, or 20 markers from Tables 2-3 or 5-9) will provide a more detailed assessment of the characteristics of the cell. Expression of genes that are down-regulated and/or lack of expression of genes that are up-regulated upon differentiation correlates with a differentiated phenotype. Expression of genes that are up-regulated and/or lack of expression of genes that are down-regulated upon differentiation correlates with an undifferentiated phenotype. The markers newly identified in this disclosure may be analyzed together (with or without markers that were previously known) in any combination effective for characterizing the cell status or phenotype.

Tissue-specific markers can be detected using any suitable immunological technique—such as flow cytochemistry for cell-surface markers, or immunocytochemistry (for example, of fixed cells or tissue sections) for intracellular or cell-surface markers. Expression of a cell-surface antigen is defined as positive if a significantly detectable amount of antibody will bind to the antigen in a standard immunocytochemistry or flow cytometry assay, optionally after fixation of the cells, and optionally using a labeled secondary antibody or other conjugate to amplify labeling.

The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods. See U.S. Pat. No. 5,843,780 for further details. Sequence data for particular markers listed in this disclosure can be obtained from public databases such as GenBank.

These and other suitable assay systems are described in standard reference texts, such as the following: PCR Cloning Protocols, 2^{nd} Ed (James & Chen eds., Humana Press, 2002); Rapid Cycle Real-Time PCR: Methods and Applications (C. Wittwer et al. eds., Springer-Verlag NY, 2002); Immunoassays: A Practical Approach (James Gosling ed., Oxford Univ Press, 2000); Cytometric Analysis of Cell Phenotype and Function (McCarthy et al. eds., Cambridge Univ Press, 2001). Reagents for conducting these assays, such as nucleotide probes or primers, or specific antibody, can be packaged in kit form, optionally with instructions for the use of the reagents in the characterization or monitoring of pPS cells, or their differentiated progeny.

### Use of Cell Markers for Clinical Diagnosis

Stem cells regulate their own replenishment and serve as a source of cells that can differentiate into defined cell lineages. Cancer cells also have the ability to self-renew, but lack of regulation results in uncontrolled cellular proliferation. Three key signaling pathways, Wnt, Sonic hedgehog (Shh), and Notch, are known growth regulators of tumor cells. The genomics data provided in this disclosure indicate that all three of these pathways are active in hES cells.

It is a hypothesis of this invention that many of the markers discovered to be more highly expressed in undifferentiated pPS cells can also be up-regulated upon dedifferentiation of cells upon malignant transformation. Accordingly, this disclosure provides a system for evaluating clinical conditions associated with abnormal cell growth, such as hyperplasia or cancers of various kinds. Markers meeting the desired criteria include those contained in Tables 2, 5, 7 and 9.

Expression of each marker of interest is determined at the mRNA or protein level using a suitable assay system such as those described earlier; and then the expression is correlated with the clinical condition that the patient is suspected of having. As before, combinations of multiple markers may be more effective in doing the assessment. Presence of a particular marker may also provide a means by which a toxic agent or other therapeutic drug may be targeted to the disease site.

In a similar fashion, the markers of this invention can be used to evaluate a human or non-human subject who has been treated with a cell population or tissue generated by differentiating pPS cells. A histological sample taken at or near the site of administration, or a site to which the cells would be expected to migrate, could be harvested at a time subsequent to treatment, and then assayed to assess whether any of the administered cells had reverted to the undifferentiated phenotype. Reagents for conducting diagnostic tests, such as nucleotide probes or primers, or specific antibody, can be packaged in kit form, optionally with instructions for the use of the reagents in the determination of a disease condition.

### Use of Cell Markers to Assess and Manipulate Culture Conditions

The markers and marker combinations of this invention provide a system for monitoring undifferentiated pPS cells and their differentiated progeny in culture. This system can be used as a quality control, to compare the characteristics of undifferentiated pPS cells between different passages or different batches. It can also be used to assess a change in culture conditions, to determine the effect of the change on the undifferentiated cell phenotype.

Where the object is to produce undifferentiated cells, a decrease in the level of expression of an undifferentiated marker because of the alteration by 3-, 10-, 25-, 100- and 1000-fold is progressively less preferred. Corresponding increases in marker expression may be more beneficial. Moderate decreases in marker expression may be quite acceptable within certain boundaries, if the cells retain their ability to form progeny of all three germ layers is retained, and/or the level of the undifferentiated marker is relatively restored when culture conditions are returned to normal.

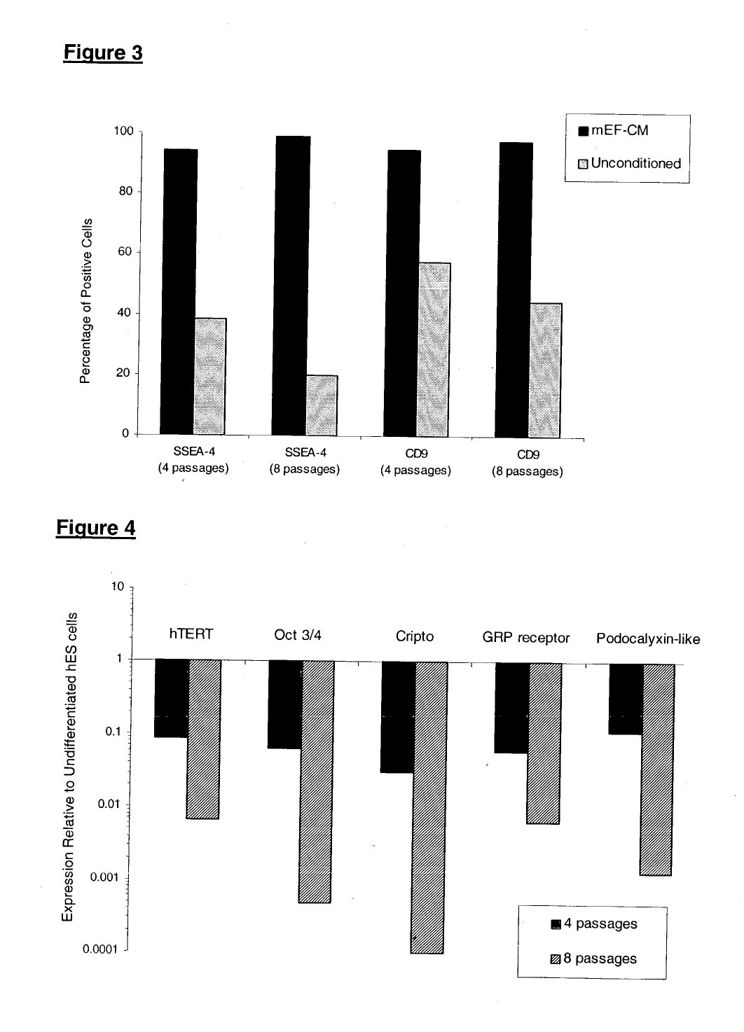

In this manner, the markers of this invention can be used to evaluate different feeder cells, extracellular matrixes, base media, additives to the media, culture vessels, or other features of the culture as illustrated in WO 99/20741 and PCT application PCT/US02/28200. Illustrations of this technique are provided below in Example 6 (FIGS. 3 to 6).

In a similar fashion, the markers of this invention can also be used to monitor and optimize conditions for differentiating cells. Improved differentiation procedures will lead to higher or more rapid expression of markers for the differentiated phenotype, and/or lower or more rapid decrease in expression of markers for the undifferentiated phenotype.

### Use of Cell Markers to Regulate Gene Expression

Differential expression of the markers listed in this disclosure indicates that each marker is controlled by a transcriptional regulatory element (such as a promoter) that is tissue specific, causing higher levels of expression in undifferentiated cells compared with differentiated cells, or vice versa. When the corresponding transcriptional regulatory element is combined with a heterologous encoding region to drive expression of the encoding region, then the expression pattern in different cell types will mimic that of the marker gene.

Minimum promoter sequences of many of the genes listed in this disclosure are known and further described elsewhere. Where a promoter has not been fully characterized, specific transcription can usually be driven by taking the 500 base pairs immediately upstream of the translation start signal for the marker in the corresponding genomic clone.

To express a heterologous encoding region according to this embodiment of the invention, a recombinant vector is constructed in which the specific promoter of interest is operatively linked to the encoding region in such a manner that it drives transcription of the encoding region upon transfection into a suitable host cell. Suitable vector systems for transient expression include those based on adenovirus and certain types of plasmids. Vectors for long-term expression include those based on plasmid lipofection or electroporation, episomal vectors, retrovirus, and lentivirus.

One application of tissue-specific promoters is expression of a reporter gene. Suitable reporters include fluorescence markers such as green fluorescent protein, luciferase, or enzymatic markers such as alkaline phosphatase and β-galactosidase. Other reporters such as a blood group glycosyltransferase (WO 02/074935), or Invitrogen's pDisplay™, create a cell surface epitope that can be counterstained with labeled specific antibody or lectin. pPS cells labeled with reporters can be used to follow the differentiation process directly, the presence or absence of the reporter correlating with the undifferentiated or differentiated phenotype, depending on the specificity of the promoter. This in turn can be used to follow or optimize culture conditions for undifferentiated pPS cells, or differentiation protocols. Alternatively, cells containing promoter-reporter constructs can be used for drug screening, in which a test compound is combined with the cell, and expression or suppression of the promoter is correlated with an effect attributable to the compound.

Another application of tissue-specific promoters is expression of a positive or negative drug selection marker. Antibiotic resistance genes such as neomycin phosphotransferase, expressed under control of a tissue-specific promoter, can be used to positively select for undifferentiated or differentiated cells in a medium containing the corresponding drug (geneticin), by choosing a promoter with the appropriate specificity. Toxin genes, genes that mediate apoptosis, or genes that convert a prodrug into a toxic compound (such as thymidine kinase) can be used to negatively select against contaminating undifferentiated or differentiated cells in a population of the opposite phenotype (WO 02/42445; GB 2374076).

Promoters specific for the undifferentiated cell phenotype can also be used as a means for targeting cancer cells—using the promoter to drive expression of a gene that is toxic to the cell (WO 98/14593, WO 02/42468), or to drive a replication gene in a viral vector (WO 00/46355). For example, an adenoviral vector in which the GRPR promoter (AY032865) drives the E1a gene should specifically lyse cancer cells in the manner described in Majumdar et al., Gene Ther. 8:568, 2001. Multiple promoters for the undifferentiated phenotype can be linked for improved cancer specificity (U.S. Ser. No. 10/206,447).

Other useful applications of tissue-specific promoters of this invention will come readily to the mind of the skilled reader.

### Use of Markers for Cell Separation or Purification

Differentially expressed markers provided in this disclosure are also a means by which mixed cell populations can be separated into populations that are more homogeneous. This can be accomplished directly by selecting a marker of the undifferentiated or differentiated phenotype, which is itself expressed on the cell surface, or otherwise causes expression of a unique cell-surface epitope. The epitope is then used as a handle by which the marked cells can be physically separated from the unmarked cells. For example, marked cells can be aggregated or adsorbed to a solid support using an antibody or lectin that is specific for the epitope. Alternatively, the marker can be used to attach a fluorescently labeled antibody or lectin, and then the cell suspension can be subject to fluorescence-activated cell sorting.

An alternative approach is to take a tissue-specific promoter chosen based on its expression pattern (as described in the last section), and use it to drive transcription of a gene suitable for separating the cells. In this way, the marker from which the promoter is chosen need not itself be a cell surface protein. For example, the promoter can drive expression of a fluorescent gene, such as GFP, and then cells having the marked phenotype can be separated by FACS. In another example, the promoter drives expression of a heterologous gene that causes expression of a cell-surface epitope. The epitope is then used for adsorption-based separation, or to attach a fluorescent label, as already described.

### Use of Cell Markers to Influence Differentiation

In another embodiment of this invention, the differentially expressed genes of this invention are caused to increase or decrease their expression level, in order to either inhibit or promote the differentiation process. Suitable genes are those that are believed in the normal case of ontogeny to be active in maintaining the undifferentiated state, active in the general process of differentiation, or active in differentiation into particular cell lineages. Markers of interest for this application are the following:

Transcription factors and other elements that directly affect transcription of other genes, such as Forkhead box O1A (FOXO1A); Zic family member 3 (ZIC3); Hypothetical protein FLJ20582; Forkhead box H1 (FOXH1); Zinc finger protein, Hsal2; KRAB-zinc finger protein SZF1-1; Zinc finger protein of cerebellum ZIC2; and Coup transcription factor 2 (COUP-TF2). Other candidates include those marked in Tables 5 and 6 with the symbol “\mathring{x}”, and other factors with zinc fingers or nucleic acid binding activity.

Genes that influence cell interaction, such as those that encode adhesion molecules, and enzymes that make substrates for adhesion molecules

Genes encoding soluble factors that transmit signals within or between cells, and specific receptors that recognize them and are involved in signal transduction.

One way of manipulating gene expression is to induce a transient or stable genetic alteration in the cells using a suitable vector, such as those already listed. Scientists at Geron Corp. have determined that the following constitutive promoters are effective in undifferentiated hES cells: for transient expression CMV, SV40, EF1α, UbC, and PGK; for stable expression, SV40, EF1α, UbC, MND and PGK. Expressing a gene associated with the undifferentiated phenotype may assist the cells to stay undifferentiated in the absence of some of the elements usually required in the culture environment. Expressing a gene associated with the differentiated phenotype may promote early differentiation, and/or initiate a cascade of events beneficial for obtaining a desired cell population. Maintaining or causing expression of a gene of either type early in the differentiation process may in some instances help guide differentiation down a particular pathway.

Another way of manipulating gene expression is to alter transcription from the endogenous gene. One means of accomplishing this is to introduce factors that specifically influence transcription through the endogenous promoter. Another means suitable for down-regulating expression at the protein level is to genetically alter the cells with a nucleic acid that removes the mRNA or otherwise inhibits translation (for example, a hybridizing antisense molecule, ribozyme, or small interfering RNA). Dominant-negative mutants of the target factor can reduce the functional effect of the gene product. Targeting a particular factor associated with the undifferentiated phenotype in this fashion can be used to promote differentiation. In some instances, this can lead to de-repression of genes associated with a particular cell type.

Where the gene product is a soluble protein or peptide that influences cell interaction or signal transduction (for example, cytokines like osteopontin and Cripto), then it may be possible to affect differentiation simply by adding the product to the cells—in either recombinant or synthetic form, or purified from natural sources. Products that maintain the undifferentiated phenotype can then be withdrawn from the culture medium to initiate differentiation; and products that promote differentiation can be withdrawn once the process is complete.

Since differentiation is a multi-step process, changing the level of gene product on a permanent basis may cause multiple effects. In some instances, it may be advantageous to affect gene expression in a temporary fashion at each sequential step in the pathway, in case the same factor plays different effects at different steps of differentiation. For example, function of transcription factors can be evaluated by changing expression of individual genes, or by invoking a high throughput analysis, using cDNAs obtained from a suitable library such as exemplified in Example 1. Cells that undergo an alteration of interest can be cloned and pulled from multi-well plates, and the responsible gene identified by PCR amplification.

The effect of up- or down-regulating expression of a particular gene can be determined by evaluating the cell for morphological characteristics, and the expression of other characteristic markers. Besides the markers listed later in this disclosure, the reader may want to follow the effect on particular cell types, using markers for later-stage or terminally differentiated cells. Tissue-specific markers suitable for this purpose are listed in WO 01/81549 (hepatocytes), WO 01/88104 (neural cells), PCT/US02/20998 (osteoblasts and mesenchymal cells), PCT/US02/22245 (cardiomyocytes), PCT/US02/39091 (hematopoietic cells), PCT/US02/39089 (islet cells), and PCT/US02/39090 (chondrocytes). Such markers can be analyzed by PCR amplification, fluorescence labeling, or immunocytochemistry, as already described. Promoter-reporter constructs based on the same markers can facilitate analysis when expression is being altered in a high throughput protocol.

The examples that follow are provided for further illustration, and are not meant to limit the claimed invention.

### EXAMPLES

### Example 1

### An EST Database of Undifferentiated hES Cells and Their Differentiated Progeny

cDNA libraries were prepared from human embryonic stem (hES) cells cultured in undifferentiated form. cDNA libraries were also prepared from progeny, subject to non-specific differentiation as embryoid bodies (EBs), or taken through the preliminary stages of established differentiation protocols for neurons (preNEU) or hepatocytes (preHEP).

The hES cell lines H1, H7, and H9 were maintained under feeder-free conditions. Cultures were passaged every 5-days by incubation in 1 mg/mL collagenase IV for 5-10 min at 37° C., dissociated and seeded in clumps at 2.5 to 10×10⁵ cells/well onto Matrigel™-coated six well plates in conditioned medium supplemented with 8 mg/mL bFGF. cDNA libraries were made after culturing for 5 days after the last passage.

EBs were prepared as follows. Confluent plates of undifferentiated hES cells were treated briefly with collagenase IV, and scraped to obtain small clusters of cells. Cell clusters were resuspended in 4 mL/well differentiation medium (KO DMEM containing 20% fetal bovine serum in place of 20% SR, and not preconditioned) on low adhesion 6-well plates (Costar). After 4 days in suspension, the contents of each well was transferred to individual wells pre-coated with gelatin. Each well was re-fed with 3 mL fresh differentiation medium every two days after replating. Cells were used for the preparation of cytoplasmic RNA on the eighth day after plating.

PreHEP cells were prepared based on the hepatocyte differentiation protocol described in WO 01/81549. Confluent wells of undifferentiated cells were prepared, and medium was changed to KO DMEM plus 20% SR+1% DMSO. The medium was changed every 24 h, and cells were used for preparation of cytoplasmic RNA on day 5 of DMSO treatment.

PreNEU cells were prepared based on the neural differentiation protocol described in WO 01/88104. hES cells of the H7 line (p29) were used to generate EBs as described above except that 10 μM all-trans RA was included in the differentiation medium. After 4 days in suspension, EBs were transferred to culture plate precoated with poly-L-lysine and laminin. After plating, the medium was changed to EPFI medium. Cells were used for the preparation of cytoplasmic RNA after 3 days of growth in EPFI.

Partial 5′ end sequences (an expressed sequence tag, or EST) were determined by conventional means for independent clones derived from each cDNA library. Overlapping ESTs were assembled into conjoined sequences.

TABLE 1
Non-redundant EST sequences

| | Number |
|---|---|
| Library | of ESTs |
| hESC | 37,081 |
| EB | 37,555 |
| preHEP | 35,611 |
| preNEU | 38,206 |
| Total | 148,453 |

All of the stem cell lines used for preparation of the expression libraries were originally isolated and initially propagated on mouse feeder cells. Accordingly, the libraries were analyzed to determine whether they were contaminated with murine retroviruses that had shed from the feeder cells and subsequently infected the stem cells. Three complete viral genomes were used in a BLAST search: Moloney murine leukemia virus, Friend murine leukemia virus, and murine type C retrovirus. No matches with a high score were found against any of the ESTs.

The sequences were then compared to the Unigene database of human genes. ESTs that were at least 98% identical, over a stretch of at least 150 nucleotides each, to a common reference sequence in Unigene, were assumed to be transcribed from the same gene, and placed into a common assembly. The complete set of 148,453 ESTs collapsed to a non-redundant set of 32,764 assemblies.

### Example 2

### Selection of Marker Genes Specific for Undifferentiated and Differentiated Cells

Candidate markers were selected from a database based on the imputed level of gene expression. The frequency of ESTs for any particular gene correlates with the abundance of that mRNA in the cells used to generate the cDNA library. Thus, a comparison of frequencies of ESTs among the libraries indicates the relative abundance of the associated mRNA in the different cell types.

Candidate molecular markers were selected from the expressed gene (EST) database from their greater abundance in undifferentiated hES cells, relative to differentiated hES cells. Genes were identified as having a differential expression pattern (being up- or down-regulated) during the differentiation process, if the count of ESTs sequenced in the undifferentiated cells was substantially different from the sum of ESTs in the three differentiated libraries.

Oct 3/4 (a POU domain-containing transcription factor) and telomerase reverse transcriptase (hTERT) are known to be expressed preferentially in undifferentiated hES cells (WO 01/51616). Other genes suitable for characterizing or manipulating the undifferentiated phenotype are those that are down-regulated upon differentiation with a significance of p≦0.05, as determined by the Fisher Exact Test (explained below). 193 genes were found to have 4-fold more ESTs in hES cells, relative to each of the three cell types. 532 genes were found that were 2-fold greater hES cells, with a confidence of over 95% as determined by the Fisher Exact Test, relative to the sum of ESTs of the three cell types (minimum of 4 ESTs in hES cells). The following markers are of particular interest:

TABLE 2
EST Frequency of Genes that are Down-regulated upon Differentiation of hES cells

| | | | EST counts | | | |
|---|---|---|---|---|---|---|
| Geron ID | GenBank ID | Name | ES | EB | preHEP | preNEU |
| GA_10902 | NM_024504 | Pr domain containing 14 (PRDM14) | 12 | 1 | 0 | 0 |
| GA_11893 | NM_032805 | Hypothetical protein FLJ14549 | 25 | 0 | 0 | 0 |
| GA_12318 | NM_032447 | Fibrillin3 | 6 | 0 | 0 | 0 |
| GA_1322 | NM_000142 | Fibroblast growth factor receptor 3 precursor | 9 | 1 | 5 | 1 |
| | | (FGFR-3) | | | | |
| GA_34679 | NM_002015 | Forkhead box o1a (FOXO1a) | 4 | 0 | 1 | 1 |
| GA_1470 | NM_003740 | potassium channel, subfamily K, member 5 | 4 | 0 | 0 | 1 |
| | | (KCNK5), mRNA | | | | |
| GA_1674 | NM_002701 | Octamer-Binding Transcription Factor 3a | 24 | 1 | 2 | 0 |
| | | (OCT-3A) (OCT-4) | | | | |
| GA_2024 | NM_003212 | Teratocarcinoma-derived growth factor 1 | 20 | 1 | 0 | 0 |
| | | (CRIPTO) | | | | |
| GA_2149 | NM_003413 | Zic family member 3 (ZIC3) | 7 | 0 | 1 | 0 |
| GA_2334 | NM_000216 | Kallmann syndrome 1 sequence (KAL1) | 5 | 0 | 1 | 0 |
| GA_23552 | NM_152742 | hypothetical protein DKFZp547M109 | 6 | 0 | 1 | 2 |
| | | (DKFZp547M109), mRNA | | | | |
| GA_2356 | NM_002851 | Protein tyrosine phosphatase, receptor-type, | 10 | 0 | 0 | 0 |
| | | z polypeptide 1 (PTPRZ1), | | | | |
| GA_2357 | NM_001670 | Armadillo repeat protein deleted in | 6 | 0 | 0 | 0 |
| | | velo-cardio-facial syndrome (ARVCF) | | | | |
| GA_23578 | BM454360 | AGENCOURT_6402318 NIH_MGC_85 | 6 | 0 | 0 | 0 |
| | | Homo sapiens cDNA clone IMAGE: 5497491 | | | | |
| | | 5′, mRNA sequence | | | | |
| GA_2367 | NM_003923 | Forkhead box H1 (FOXH1) | 5 | 0 | 0 | 0 |
| GA_2436 | NM_004329 | Bone morphogenetic protein receptor, type la | 7 | 3 | 1 | 1 |
| | | (BMPR1A) (ALK-3) | | | | |
| GA_2442 | NM_004335 | Bone marrow stromal antigen 2 (BST-2) | 13 | 0 | 2 | 3 |
| GA_2945 | NM_005232 | Ephrin type-a receptor 1 (EPHA1) | 5 | 1 | 1 | 1 |
| GA_2962 | NM_005314 | Gastrin-releasing peptide receptor (GRP-R) | 4 | 0 | 0 | 0 |
| GA_2988 | NM_005397 | Podocalyxin-like (PODXL) | 59 | 23 | 5 | 8 |
| GA_3337 | NM_006159 | NELL2 (nel-like protein 2) | 5 | 3 | 2 | 0 |
| GA_3559 | NM_005629 | Solute carrier family 6, member 8 (SLC6A8) | 5 | 1 | 0 | 1 |
| GA_3898 | NM_006892 | DNA (cytosine-5-)-methyltransferase 3 beta | 49 | 2 | 3 | 1 |
| | | (DNMT3B) | | | | |
| GA_5391 | NM_002968 | Sal-like 1 (SALL1), | 7 | 1 | 1 | 0 |
| GA_33680 | NM_016089 | Krab-zinc finger protein SZF1-1 | 15 | 0 | 1 | 0 |
| GA_36977 | NM_020927 | KIAA1576 protein | 9 | 2 | 1 | 0 |
| GA_8723 | NM_152333 | Homo sapiens chromosome 14 open reading | 14 | 1 | 1 | 3 |
| | | frame 69 (C14orf69), mRNA | | | | |
| GA_9167 | AF308602 | Notch 1 (N1) | 6 | 2 | 1 | 0 |
| GA_9183 | NM_007129 | Homo sapiens Zic family member 2 (odd- | 8 | 1 | 1 | 0 |
| | | paired homolog, Drosophila) (ZIC2), mRNA | | | | |
| GA_35037 | NM_004426 | Homo sapiens polyhomeotic-like 1 | 34 | 9 | 5 | 4 |
| | | (Drosophila) (PHC1), mRNA | | | | |

Only one EST for hTERT was identified in undifferentiated hES cells and none were detected from the differentiated cells, which was not statistically significant. Thus, potentially useful markers that are expressed at low levels could have been omitted in this analysis, which required a minimum of four ESTs. It would be possible to identify such genes by using other techniques described elsewhere in this disclosure.

Three genes were observed from EST frequency queries that were of particular interest as potentially useful markers of hES cells. They were Teratocarcinoma-derived growth factor (Cripto), Podocalyxin-like (PODXL), and gastrin-releasing peptide receptor (GRPR). These genes were not only more abundant in undifferentiated cells, relative to differentiated hES cells, but also encoded for proteins expressed on the surface of cells. Surface markers have the added advantage that they could be easily detected with immunological reagents. ESTs for Cripto and GRPR were quite restricted to hES cells, with one or zero ESTs, respectively, scored in any of the differentiated cells. PODXL ESTs were detected in all 4-cell types, but substantially fewer (2.5×-12×) in differentiated cells. All three markers retained a detectable level of expression in differentiated cultures of hES cells. There may be a low level of expression of these markers in differentiated cells, or the expression detected may be due to a small proportion of undifferentiated cells in the population. GABA(A) receptor, Lefty B, Osteopontin, Thy-1 co-transcribed, and Solute carrier 21 are other significant markers of the undifferentiated phenotype.

By similar reasoning, genes that show a higher frequency of ESTs in differentiated cells can be used as specific markers for differentiation. ESTs that are 2-fold more abundant in the sum of all three differentiated cell types (EBs, preHEP and preNEU cells) and with a p-value<0.05 as determined by the Fisher Exact Test, compared with undifferentiated hES cells are candidate markers for differentiation down multiple pathways. ESTs that are relatively abundant in only one of the differentiated cell types are candidate markers for tissue-specific differentiation. The following markers are of particular interest:

TABLE 3
EST Frequency of Genes that are Upregulated upon Differentiation

| | | | EST counts | | | |
|---|---|---|---|---|---|---|
| Geron ID | GenBank ID | Name | ES | EB | preHEP | preNEU |
| GA_35463 | NM_024298 | Homo sapiens leukocyte receptor cluster (LRC) | 0 | 4 | 9 | 8 |
| | | member 4 (LENG4), mRNA | | | | |
| GA_10492 | NM_006903 | Inorganic pyrophosphatase (PPASE) | 0 | 5 | 5 | 6 |
| GA_38563 | NM_021005 | Homo sapiens nuclear receptor subfamily 2, | 0 | 9 | 8 | 9 |
| | | group F, member 2 (NR2F2), mRNA | | | | |
| GA_38570 | NM_001844 | Collagen, type II, alpha 1 (COL2A1), transcript | | 15 | 31 | 5 |
| | | variant 1 | | | | |
| GA_1476 | NM_002276 | Keratin type I cytoskeletal 19 (cytokeratin 19) | 1 | 26 | 14 | 38 |
| GA_34776 | NM_002273 | Keratin type II cytoskeletal 8 (cytokeratin 8) | 9 | 71 | 144 | 156 |
| | | (CK 8) | | | | |
| GA_1735 | NM_002806 | Homo sapiens proteasome (prosome, | 1 | 7 | 7 | 8 |
| | | macropain) 26S subunit, ATPase, 6 (PSMC6), | | | | |
| | | mRNA | | | | |
| GA_1843 | NM_000982 | 60 s ribosomal protein I21 | 1 | 7 | 48 | 42 |
| GA_35369 | NM_003374 | Voltage-dependent anion-selective channel | 1 | 5 | 6 | 10 |
| | | (VDAC-1) | | | | |
| GA_23117 | NM_004772 | P311 protein [Homo sapiens] | 1 | 5 | 7 | 6 |
| GA_2597 | NM_138610 | Homo sapiens H2A histone family, member Y | 1 | 5 | 5 | 14 |
| | | (H2AFY), transcript variant 3, mRNA | | | | |
| GA_3283 | NM_004484 | Homo sapiens glypican 3 (GPC3), mRNA | 1 | 6 | 7 | 12 |
| GA_3530 | NM_002539 | Homo sapiens ornithine decarboxylase 1 | 1 | 10 | 8 | 9 |
| | | (ODC1), mRNA | | | | |
| GA_4145 | NM_002480 | Protein phosphatase 1, regulatory(inhibitor) | 1 | 6 | 6 | 6 |
| | | subunit 12A (PPP1R12A) | | | | |
| GA_5992 | NM_014899 | Homo sapiens Rho-related BTB domain | 0 | 10 | 7 | 13 |
| | | containing 3 (RHOBTB3), mRNA | | | | |
| GA_6136 | NM_016368 | Homo sapiens myo-inositol 1-phosphate | 1 | 7 | 5 | 16 |
| | | synthase A1 (ISYNA1), mRNA | | | | |
| GA_6165 | NM_015853 | Orf (LOC51035) | 1 | 5 | 9 | 5 |
| GA_6219 | NM_016139 | 16.7 Kd protein (LOC51142), | 1 | 5 | 13 | 14 |
| GA_723 | NM_005801 | Homo sapiens putative translation initiation | 1 | 14 | 15 | 19 |
| | | factor (SUI1), mRNA | | | | |
| GA_9196 | NM_000404 | Homo sapiens galactosidase, beta 1 (GLB1), | 0 | 6 | 10 | 7 |
| | | transcript variant 179423, mRNA | | | | |
| GA_9649 | NM_014604 | Tax interaction protein 1 (TIP-1) | 0 | 8 | 5 | 5 |

The relative expression levels were calculated as follows:

$$\begin{matrix}es = \frac{( \# ESTs of the gene in hES cells \div total unique genes in hES cells )}{( \# ESTs of the gene in differentiated cells \div total unique genes in differentiated cells )} \\ = \frac{( \# ESTs for the gene in hES cells \div 37 \text{,} 081 )}{( \# ESTs for the gene in differentiated cells \div 111 \text{,} 372 )}\end{matrix}$$

The es value is substantially >1 for genes marking the undifferentiated phenotype, and <1 for genes indicating differentiation.

The Fisher Exact Test was used to determine whether changes were statistically significant. S. Siegel & N. J. Castellan. Nonparametric Statistics for the Behavioral Sciences (2nd ed., McGraw-Hill NJ, 1988). This is a standard test that can be used for 2×2 tables, and is conservative in declaring significance if the data are sparse. For analysis of EST sequences, the tables were of the following form:

TABLE 4
Fisher Exact Test for Statistical Analysis of Differential Expression

| | Gene X | All Other Genes | Total |
|---|---|---|---|
| Pool | a = number of | A = number of sequences | N = a + A |
| A | sequences in Pool A | in Pool A NOT assigned | total number of |
| | assigned to Gene X | to Gene X | sequences in Pool |
| | | | A |
| Pool | b = number of | B = number of sequences | M = b + B |
| B | sequences in Pool B | in Pool B NOT assigned | total number of |
| | assigned to Gene X | to Gene X | sequences in Pool |
| | | | B |
| Total | c = a + b | C = A + B | N + M = c + C |

where Pool A contains the sequences derived from the undifferentiated hES cells and Pool B contains the sequences from the other three cell types (EB, preHep, preNeu). N is equal to the number of sequences derived from the undifferentiated hES cells (37,081) and M is equal to the sum of all ESTs from the three differentiated cell types (111,372). For any given pair of pool sizes (N, M) and gene counts (c and C), the probability p of the table being generated by chance is calculated where:

p=[N! M! c! C!]/[(N+M)! a! b! A! B!]

and where 0! by default is set to 1. The null hypothesis of a gene being equally represented in two pools is rejected when probability p≦0.05, where 0.05 is the level of statistical certainty. Thus, genes with p≦0.05 are considered to be differentially represented.

The following markers were identified as changing their expression levels significantly upon differentiation. The markers identified with the symbol “\mathring{x}” may play a role in the regulation of gene transcription.

TABLE 5
EST Frequency of Genes that Down-regulate upon Differentiation

| | | | | EST counts | | | | | | |
|---|---|---|---|---|---|---|---|---|---|---|
| Geron ID | GenBank ID | Name | | ES | EB | preHEP | preNeu | Total | Relative Expression | |
| GA_10021 | NM_018124 | hypothetical protein FLJ10520 (FLJ10520) | | | 1 | 0 | 3 | 10 | es 4.51 | p = 0.02 |
| GA_10053 | NM_033427 | cortactin binding protein 2 (CORTBP2) | | 4 | 0 | 0 | 0 | 4 | es > 4 | p = 0.00 |
| GA_10057 | AB051540 | KIAA1753 protein sequence | | 4 | 1 | 1 | 0 | 6 | es 6.01 | p = 0.04 |
| GA_10082 | NM_030645 | KIAA1720 protein (KIAA1720) | | 6 | 0 | 1 | 0 | 7 | es 18.02 | p = 0.00 |
| GA_10153 | NM_015039 | chromosome 1 open reading frame 15 (C1orf15), | | 4 | 1 | 1 | 0 | 6 | es 6.01 | p = 0.04 |
| | | transcript variant 1 | | | | | | | | |
| GA_102 | NM_015043 | KIAA0676 protein (KIAA0676) | | 6 | 4 | 0 | 1 | 11 | es 3.60 | p = 0.03 |
| GA_10252 | NM_003376 | vascular endothelial growth factor (VEGF) | | 5 | 2 | 0 | 2 | 9 | es 3.75 | p = 0.05 |
| GA_10258 | AK091948 | cDNA FLJ34629 fis, clone KIDNE2015515, highly | | 4 | 0 | 0 | 0 | 4 | es > 4 | p = 0.00 |
| | | similar to NADP-dependent leukotriene b4 12- | | | | | | | | |
| | | hydroxydehydrogenase (EC 1.1.1.-) sequence | | | | | | | | |
| GA_10308 | NM_024046 | hypothetical protein MGC8407 (MGC8407) | | 4 | 0 | 0 | 0 | 4 | es > 4 | p = 0.00 |
| GA_10327 | NM_024077 | SECIS binding protein 2 (SBP2) | | 9 | 2 | 3 | 2 | 16 | es 3.86 | p = 0.01 |
| GA_10334 | NM_024090 | long-chain fatty-acyl elongase (LCE) | | 5 | 0 | 0 | 2 | 7 | es 7.51 | p = 0.01 |
| GA_10513 | NM_033209 | Thy-1 co-transcribed (LOC94105) | | 7 | 2 | 2 | 1 | 12 | es 4.20 | p = 0.01 |
| GA_10528 | NM_030622 | cytochrome P450, subfamily IIS, polypeptide 1 | | 6 | 0 | 1 | 0 | 7 | es 18.02 | p = 0.00 |
| | | (CYP2S1) | | | | | | | | |
| GA_1053 | NM_001618 | ADP-ribosyltransferase (NAD+; poly (ADP-ribose) | | 25 | 13 | 14 | 9 | 61 | es 2.09 | p = 0.01 |
| | | polymerase) (ADPRT) | | | | | | | | |
| GA_10531 | NM_015271 | tripartite motif-containing 2 (TRIM2) | | 6 | 2 | 0 | 2 | 10 | es 4.51 | p = 0.02 |
| GA_10603 | NM_025215 | pseudouridylate synthase 1 (PUS1) | | 5 | 0 | 2 | 2 | 9 | es 3.75 | p = 0.05 |
| GA_10641 | NM_025108 | hypothetical protein FLJ13909 (FLJ13909) | | 6 | 0 | 0 | 1 | 7 | es 18.02 | p = 0.00 |
| GA_10649 | NM_025082 | hypothetical protein FLJ13111 (FLJ13111) | | 8 | 3 | 0 | 0 | 11 | es 8.01 | p = 0.00 |
| GA_1067 | NM_020977 | ankyrin 2, neuronal (ANK2), transcript variant 2 | | 4 | 0 | 0 | 0 | 4 | es > 4 | p = 0.00 |
| GA_10696 | NM_024888 | hypothetical protein FLJ11535 (FLJ11535) | | 5 | 2 | 0 | 0 | 7 | es 7.51 | p = 0.01 |
| GA_10713 | NM_024844 | pericentrin 1 (PCNT1) | | 8 | 1 | 1 | 0 | 10 | es 12.01 | p = 0.00 |
| GA_1076 | NM_001659 | ADP-ribosylation factor 3 (ARF3) | | 19 | 8 | 5 | 4 | 36 | es 3.36 | p = 0.00 |
| GA_10831 | NM_024619 | hypothetical protein FLJ12171 (FLJ12171) | | 4 | 0 | 1 | 1 | 6 | es 6.01 | p = 0.04 |
| GA_1085 | NM_000048 | argininosuccinate lyase (ASL) | | 6 | 2 | 0 | 0 | 8 | es 9.01 | p = 0.00 |
| GA_10902 | NM_024504 | PR domain containing 14 (PRDM14) | | 12 | 1 | 0 | 0 | 13 | es 36.04 | p = 0.00 |
| GA_10905 | NM_022362 | MMS19-like (MET18 homolog, S. cerevisiae) | | 10 | 5 | 4 | 1 | 20 | es 3.00 | p = 0.02 |
| | | (MMS19L) | | | | | | | | |
| GA_10935 | NM_032569 | cytokine-like nuclear factor n-pac (N-PAC) | | 8 | 3 | 1 | 1 | 13 | es 4.81 | p = 0.01 |
| GA_11047 | NM_004728 | DEAD/H (Asp-Glu-Ala-Asp/His) box polypeptide 21 | | 18 | 9 | 3 | 5 | 35 | es 3.18 | p = 0.00 |
| | | (DDX21) | | | | | | | | |
| GA_11103 | NM_138347 | hypothetical protein BC005868 (LOC90233) | | 4 | 0 | 2 | 0 | 6 | es 6.01 | p = 0.04 |
| GA_1119 | NM_001217 | carbonic anhydrase XI (CA11) | | 5 | 1 | 2 | 1 | 9 | es 3.75 | p = 0.05 |
| GA_11368 | NM_032147 | hypothetical protein DKFZp434D0127 | | 7 | 1 | 0 | 0 | 8 | es 21.02 | p = 0.00 |
| | | (DKFZP434D0127) | | | | | | | | |
| GA_11398 | NM_015471 | DKFZP566O1646 protein (DC8) | | 5 | 1 | 1 | 0 | 7 | es 7.51 | p = 0.01 |
| GA_11528 | NM_021633 | kelch-like protein C3IP1 (C3IP1) | | 5 | 1 | 0 | 1 | 7 | es 7.51 | p = 0.01 |
| GA_11532 | NM_024900 | PHD protein Jade-1 (Jade-1) | [symbol] | 6 | 1 | 0 | 2 | 9 | es 6.01 | p = 0.01 |
| GA_11552 | NM_024086 | hypothetical protein MGC3329 (MGC3329) | | 6 | 3 | 0 | 1 | 10 | es 4.51 | p = 0.02 |
| GA_11577 | AB058780 | KIAA1877 protein sequence | | 4 | 2 | 0 | 0 | 6 | es 6.01 | p = 0.04 |
| GA_1160 | NM_052988 | cyclin-dependent kinase (CDC2-like) 10 (CDK10), | | 4 | 0 | 1 | 1 | 6 | es 6.01 | p = 0.04 |
| | | transcript variant 3 | | | | | | | | |
| GA_11600 | NM_002883 | Ran GTPase activating protein 1 (RANGAP1) | | 12 | 7 | 3 | 5 | 27 | es 2.40 | p = 0.03 |
| GA_11656 | NM_018425 | phosphatidylinositol 4-kinase type II (PI4KII) | | 5 | 1 | 1 | 2 | 9 | es 3.75 | p = 0.05 |
| GA_11773 | NM_025109 | hypothetical protein FLJ22865 (FLJ22865) | | 6 | 0 | 0 | 0 | 6 | es > 4 | p = 0.00 |
| GA_11790 | NM_013432 | nuclear factor of kappa light polypeptide gene | [symbol] | 5 | 2 | 0 | 0 | 7 | es 7.51 | p = 0.01 |
| | | enhancer in B-cells inhibitor-like 2 (NFKBIL2) | | | | | | | | |
| GA_11868 | NM_032844 | hypothetical protein FLJ14813 (FLJ14813) | | 6 | 2 | 1 | 1 | 10 | es 4.51 | p = 0.02 |
| GA_11893 | NM_032805 | hypothetical protein FLJ14549 (FLJ14549) | | 25 | 0 | 0 | 0 | 25 | es > 4 | p = 0.00 |
| GA_11964 | NM_032620 | mitochondrial GTP binding protein (GTPBG3) | | 5 | 1 | 1 | 2 | 9 | es 3.75 | p = 0.05 |
| GA_11971 | NM_138575 | hypothetical protein MGC5352 (MGC5352) | | 4 | 1 | 1 | 0 | 6 | es 6.01 | p = 0.04 |
| GA_12025 | NM_020465 | NDRG family member 4 (NDRG4) | | 4 | 1 | 0 | 0 | 5 | es 12.01 | p = 0.02 |
| GA_12064 | | | | 4 | 1 | 0 | 0 | 5 | es 12.01 | p = 0.02 |
| GA_1212 | NM_001313 | collapsin response mediator protein 1 (CRMP1) | | 7 | 1 | 1 | 2 | 11 | es 5.26 | p = 0.01 |
| GA_12167 | NM_138357 | hypothetical protein BC010682 (LOC90550) | | 4 | 0 | 0 | 0 | 4 | es > 4 | p = 0.00 |
| GA_1217 | NM_001316 | CSE1 chromosome segregation 1-like (yeast) | | 23 | 7 | 5 | 2 | 37 | es 4.93 | p = 0.00 |
| | | (CSE1L) | | | | | | | | |
| GA_12173 | NM_021912 | gamma-aminobutyric acid (GABA) A receptor, beta | | 4 | 0 | 0 | 0 | 4 | es > 4 | p = 0.00 |
| | | 3 (GABRB3), transcript variant 2 | | | | | | | | |
| GA_12253 | NM_032420 | protocadherin 1 (cadherin-like 1) (PCDH1), | | 5 | 0 | 0 | 2 | 7 | es 7.51 | p = 0.01 |
| | | transcript variant 2 | | | | | | | | |
| GA_12279 | NM_033019 | PCTAIRE protein kinase 1 (PCTK1), transcript | | 11 | 7 | 2 | 4 | 24 | es 2.54 | p = 0.03 |
| | | variant 3 | | | | | | | | |
| GA_12318 | NM_032447 | fibrillin3 (KIAA1776) | | 6 | 0 | 0 | 0 | 6 | es > 4 | p = 0.00 |
| GA_1236 | NM_003611 | oral-facial-digital syndrome 1 (OFD1) | | 4 | 0 | 1 | 0 | 5 | es 12.01 | p = 0.02 |
| GA_12367 | NM_033317 | hypothetical gene ZD52F10 (ZD52F10) | | 8 | 1 | 4 | 4 | 17 | es 2.67 | p = 0.05 |
| GA_12386 | AB002336 | KIAA0338 sequence | | 4 | 1 | 0 | 0 | 5 | es 12.01 | p = 0.02 |
| GA_12440 | NM_032383 | Hermansky-Pudlak syndrome 3 (HPS3) | | 7 | 1 | 0 | 0 | 8 | es 21.02 | p = 0.00 |
| GA_12522 | NM_052860 | kruppel-like zinc finger protein (ZNF300) | [symbol] | 6 | 2 | 2 | 1 | 11 | es 3.60 | p = 0.03 |
| GA_1260 | NM_000791 | dihydrofolate reductase (DHFR) | | 15 | 4 | 2 | 4 | 25 | es 4.51 | p = 0.00 |
| GA_12630 | NM_015356 | scribble (SCRIB) | | 12 | 4 | 0 | 2 | 18 | es 6.01 | p = 0.00 |
| GA_12635 | NM_002913 | replication factor C (activator 1) 1, 145 kDa (RFC1) | | 8 | 0 | 1 | 0 | 9 | es 24.03 | p = 0.00 |
| GA_12640 | NM_004741 | nucleolar and coiled-body phosphoprotein 1 | | 16 | 9 | 7 | 6 | 38 | es 2.18 | p = 0.02 |
| | | (NOLC1) | | | | | | | | |
| GA_1265 | NM_001387 | dihydropyrimidinase-like 3 (DPYSL3) | | 39 | 13 | 3 | 14 | 69 | es 3.90 | p = 0.00 |
| GA_12672 | D86976 | similar to C.elegans protein (Z37093) sequence | | 5 | 2 | 0 | 1 | 8 | es 5.01 | p = 0.03 |
| GA_12767 | NM_015360 | KIAA0052 protein (KIAA0052) | | 8 | 2 | 2 | 1 | 13 | es 4.81 | p = 0.01 |
| GA_12899 | BC039246 | clone IMAGE: 5278517 | | 5 | 2 | 1 | 1 | 9 | es 3.75 | p = 0.05 |
| GA_12900 | NM_003302 | thyroid hormone receptor interactor 6 (TRIP6) | [symbol] | 12 | 3 | 3 | 4 | 22 | es 3.60 | p = 0.00 |
| GA_12949 | BC033781 | PAX transcription activation domain interacting | [symbol] | 4 | 0 | 0 | 1 | 5 | es 12.01 | p = 0.02 |
| | | protein 1 like sequence | | | | | | | | |
| GA_12954 | NM_003972 | BTAF1 RNA polymerase II, B-TFIID transcription | [symbol] | 7 | 3 | 2 | 0 | 12 | es 4.20 | p = 0.01 |
| | | factor-associated, 170 kDa (Mot1 homolog, S. | | | | | | | | |
| | | cerevisiae) (BTAF1) | | | | | | | | |
| GA_1322 | NM_000142 | fibroblast growth factor receptor 3 (achondroplasia, | | 9 | 1 | 5 | 1 | 16 | es 3.86 | p = 0.01 |
| | | thanatophoric dwarfism) (FGFR3), transcript variant 1 | | | | | | | | |
| GA_1378 | NM_000178 | glutathione synthetase (GSS) | | 4 | 0 | 1 | 1 | 6 | es 6.01 | p = 0.04 |
| GA_1386 | NM_001517 | general transcription factor IIH, polypeptide 4 (52 kD | [symbol] | 8 | 1 | 2 | 2 | 13 | es 4.81 | p = 0.01 |
| | | subunit) (GTF2H4) | | | | | | | | |
| GA_1470 | NM_003740 | potassium channel, subfamily K, member 5 | | 4 | 0 | 0 | 1 | 5 | es 12.01 | p = 0.02 |
| | | (KCNK5) | | | | | | | | |
| GA_1523 | NM_002442 | musashi homolog 1 (Drosophila) (MSI1) | [symbol] | 4 | 1 | 0 | 0 | 5 | es 12.01 | p = 0.02 |
| GA_1529 | NM_172164 | nuclear autoantigenic sperm protein (histone- | | 58 | 7 | 32 | 15 | 112 | es 3.23 | p = 0.00 |
| | | binding) (NASP), transcript variant 1 | | | | | | | | |
| GA_1634 | NM_002647 | phosphoinositide-3-kinase, class 3 (PIK3C3) | | 5 | 1 | 1 | 2 | 9 | es 3.75 | p = 0.05 |
| GA_1650 | NM_002660 | phospholipase C, gamma 1 (formerly subtype 148) | | 10 | 4 | 4 | 1 | 19 | es 3.34 | p = 0.01 |
| | | (PLCG1) | | | | | | | | |
| GA_1662 | AF195139 | pinin (PNN) gene, complete cds | | 23 | 9 | 7 | 5 | 44 | es 3.29 | p = 0.00 |
| GA_1665 | NM_002691 | polymerase (DNA directed), delta 1, catalytic subunit | | 9 | 6 | 2 | 1 | 18 | es 3.00 | p = 0.02 |
| | | 125 kDa (POLD1) | | | | | | | | |
| GA_1674 | NM_002701 | POU domain, class 5, transcription factor 1 | [symbol] | 24 | 1 | 2 | 0 | 27 | es 24.03 | p = 0.00 |
| | | (POU5F1) | | | | | | | | |
| GA_1696 | NM_000947 | primase, polypeptide 2A, 58 kDa (PRIM2A) | | 4 | 0 | 0 | 1 | 5 | es 12.01 | p = 0.02 |
| GA_1702 | NM_002740 | protein kinase C, iota (PRKCI) | | 8 | 2 | 2 | 1 | 13 | es 4.81 | p = 0.01 |
| GA_171 | BC013923 | Similar to SRY-box containing gene 2 sequence | | 12 | 1 | 1 | 3 | 17 | es 7.21 | p = 0.00 |
| GA_1710 | NM_002764 | phosphoribosyl pyrophosphate synthetase 1 | | 7 | 3 | 2 | 1 | 13 | es 3.50 | p = 0.02 |
| | | (PRPS1) | | | | | | | | |
| GA_1752 | NM_152881 | PTK7 protein tyrosine kinase 7 (PTK7), transcript | | 15 | 14 | 5 | 3 | 37 | es 2.05 | p = 0.04 |
| | | variant 3 | | | | | | | | |
| GA_1777 | NM_002862 | phosphorylase, glycogen; brain (PYGB), nuclear | | 13 | 8 | 1 | 2 | 24 | es 3.55 | p = 0.00 |
| | | gene encoding mitochondrial protein | | | | | | | | |
| GA_1794 | NM_003610 | RAE1 RNA export 1 homolog (S. pombe) (RAE1) | | 5 | 0 | 0 | 2 | 7 | es 7.51 | p = 0.01 |
| GA_1814 | NM_002907 | RecQ protein-like (DNA helicase Q1-like) (RECQL), | | 4 | 2 | 0 | 0 | 6 | es 6.01 | p = 0.04 |
| | | transcript variant 1 | | | | | | | | |
| GA_1820 | NM_002916 | replication factor C (activator 1) 4, 37 kDa (RFC4) | | 6 | 0 | 2 | 2 | 10 | es 4.51 | p = 0.02 |
| GA_1865 | NM_002949 | mitochondrial ribosomal protein L12 (MRPL12), | | 4 | 0 | 0 | 2 | 6 | es 6.01 | p = 0.04 |
| | | nuclear gene encoding mitochondrial protein | | | | | | | | |
| GA_1909 | NM_003012 | secreted frizzled-related protein 1 (SFRP1) | | 12 | 8 | 1 | 7 | 28 | es 2.25 | p = 0.05 |
| GA_1938 | NM_003601 | SWI/SNF related, matrix associated, actin | | 19 | 10 | 4 | 5 | 38 | es 3.00 | p = 0.00 |
| | | dependent regulator of chromatin, subfamily a, | | | | | | | | |
| | | member 5 (SMARCA5) | | | | | | | | |
| GA_1942 | NM_003076 | SWI/SNF related, matrix associated, actin | | 10 | 3 | 3 | 3 | 19 | es 3.34 | p = 0.01 |
| | | dependent regulator of chromatin, subfamily d, | | | | | | | | |
| | | member 1 (SMARCD1), transcript variant 1 | | | | | | | | |
| GA_1962 | NM_152826 | sorting nexin 1 (SNX1), transcript variant 3 | | 4 | 0 | 0 | 1 | 5 | es 12.01 | p = 0.02 |
| GA_1963 | NM_003100 | sorting nexin 2 (SNX2) | | 8 | 2 | 4 | 1 | 15 | es 3.43 | p = 0.02 |
| GA_2024 | NM_003212 | teratocarcinoma-derived growth factor 1 (TDGF1) | | 20 | 1 | 0 | 0 | 21 | es 60.07 | p = 0.00 |
| GA_2031 | NM_003234 | transferrin receptor (p90, CD71) (TFRC) | | 13 | 9 | 3 | 4 | 29 | es 2.44 | p = 0.02 |
| GA_2066 | NM_003283 | troponin T1, skeletal, slow (TNNT1) | | 5 | 1 | 1 | 0 | 7 | es 7.51 | p = 0.01 |
| GA_2091 | NM_001069 | tubulin, beta polypeptide (TUBB) | | 40 | 13 | 11 | 17 | 81 | es 2.93 | p = 0.00 |
| GA_2123 | NM_003481 | ubiquitin specific protease 5 (isopeptidase T) (USP5) | | 13 | 6 | 5 | 1 | 25 | es 3.25 | p = 0.00 |
| GA_2149 | NM_003413 | Zic family member 3 heterotaxy 1 (odd-paired | [symbol] | 7 | 0 | 1 | 0 | 8 | es 21.02 | p = 0.00 |
| | | homolog, Drosophila) (ZIC3) | | | | | | | | |
| GA_2175 | NM_001605 | alanyl-tRNA synthetase (AARS) | | 23 | 6 | 1 | 3 | 33 | es 6.91 | p = 0.00 |
| GA_2178 | NM_001104 | actinin, alpha 3 (ACTN3) | | 6 | 1 | 0 | 0 | 7 | es 18.02 | p = 0.00 |
| GA_2234 | NM_000107 | damage-specific DNA binding protein 2, 48 kDa | | 8 | 1 | 0 | 2 | 11 | es 8.01 | p = 0.00 |
| | | (DDB2) | | | | | | | | |
| GA_2235 | NM_001358 | DEAD/H (Asp-Glu-Ala-Asp/His) box polypeptide 15 | | 13 | 7 | 3 | 1 | 24 | es 3.55 | p = 0.00 |
| | | (DDX15) | | | | | | | | |
| GA_2240 | NM_001384 | diptheria toxin resistance protein required for | | 6 | 1 | 2 | 0 | 9 | es 6.01 | p = 0.01 |
| | | diphthamide biosynthesis-like 2 (S. cerevisiae) | | | | | | | | |
| | | (DPH2L2) | | | | | | | | |
| GA_2271 | NM_001533 | heterogeneous nuclear ribonucleoprotein L (HNRPL) | | 10 | 1 | 4 | 5 | 20 | es 3.00 | p = 0.02 |
| GA_2289 | NM_000234 | ligase I, DNA, ATP-dependent (LIG1) | | 10 | 2 | 5 | 3 | 20 | es 3.00 | p = 0.02 |
| GA_2319 | NM_000456 | sulfite oxidase (SUOX), nuclear gene encoding | | 5 | 1 | 1 | 0 | 7 | es 7.51 | p = 0.01 |
| | | mitochondrial protein | | | | | | | | |
| GA_2323 | NM_002164 | indoleamine-pyrrole 2,3 dioxygenase (INDO) | | 6 | 0 | 0 | 0 | 6 | es > 4 | p = 0.00 |
| GA_2334 | NM_000216 | Kallmann syndrome 1 sequence (KAL1) | | 5 | 0 | 1 | 0 | 6 | es 15.02 | p = 0.00 |
| GA_2337 | NM_003501 | acyl-Coenzyme A oxidase 3, pristanoyl (ACOX3) | | 4 | 0 | 0 | 1 | 5 | es 12.01 | p = 0.02 |
| GA_23430 | NM_006474 | lung type-I cell membrane-associated glycoprotein | | 5 | 2 | 1 | 0 | 8 | es 5.01 | p = 0.03 |
| | | (T1A-2) | | | | | | | | |
| GA_23457 | AK055600 | cDNA FLJ31038 fis, clone HSYRA2000159 | | 6 | 2 | 0 | 2 | 10 | es 4.51 | p = 0.02 |
| | | sequence | | | | | | | | |
| GA_23467 | AK092578 | cDNA FLJ35259 fis, clone PROST2004251 | | 4 | 0 | 0 | 0 | 4 | es > 4 | p = 0.00 |
| | | sequence | | | | | | | | |
| GA_23468 | | | | 6 | 2 | 0 | 2 | 10 | es 4.51 | p = 0.02 |
| GA_23476 | | | | 5 | 0 | 2 | 0 | 7 | es 7.51 | p = 0.01 |
| GA_23484 | | | | 43 | 0 | 1 | 0 | 44 | es 129.15 | p = 0.00 |
| GA_23485 | | | | 25 | 1 | 1 | 0 | 27 | es 37.54 | p = 0.00 |
| GA_23486 | | | | 7 | 0 | 0 | 0 | 7 | es > 4 | p = 0.00 |
| GA_23487 | | | | 49 | 0 | 0 | 0 | 49 | es > 4 | p = 0.00 |
| GA_23488 | | | | 9 | 0 | 0 | 0 | 9 | es > 4 | p = 0.00 |
| GA_23489 | | | | 13 | 0 | 0 | 0 | 13 | es > 4 | p = 0.00 |
| GA_23490 | | | | 12 | 1 | 1 | 0 | 14 | es 18.02 | p = 0.00 |
| GA_23514 | | | | 5 | 1 | 0 | 2 | 8 | es 5.01 | p = 0.03 |
| GA_23515 | | | | 4 | 0 | 0 | 0 | 4 | es > 4 | p = 0.00 |
| GA_23525 | | | | 8 | 3 | 0 | 0 | 11 | es 8.01 | p = 0.00 |
| GA_2356 | NM_002851 | protein tyrosine phosphatase, receptor-type, Z | | 10 | 0 | 0 | 0 | 10 | es > 4 | p = 0.00 |
| | | polypeptide 1 (PTPRZ1) | | | | | | | | |
| GA_2357 | NM_001670 | armadillo repeat gene deletes in velocardiofacial | | 6 | 0 | 0 | 0 | 6 | es > 4 | p = 0.00 |
| | | syndrome (ARVCF) | | | | | | | | |
| GA_23572 | | | | 4 | 1 | 1 | 0 | 6 | es 6.01 | p = 0.04 |
| GA_23577 | | | | 4 | 2 | 0 | 0 | 6 | es 6.01 | p = 0.04 |
| GA_23578 | BM454360 | AGENCOURT_6402318 NIH_MGC_85cDNA clone | | 6 | 0 | 0 | 0 | 6 | es > 4 | p = 0.00 |
| | | IMAGE: 5497491 5′sequence | | | | | | | | |
| GA_23579 | | | | 4 | 0 | 0 | 0 | 4 | es > 4 | p = 0.00 |
| GA_23585 | | | | 8 | 0 | 1 | 1 | 10 | es 12.01 | p = 0.00 |
| GA_23596 | | | | 4 | 0 | 1 | 0 | 5 | es 12.01 | p = 0.02 |
| GA_23612 | NM_005762 | tripartite motif-containing 28 protein; KRAB- | [symbol] | 6 | 2 | 1 | 0 | 9 | es 6.01 | p = 0.01 |
| | | associated protein 1; transcriptional intermediary | | | | | | | | |
| | | factor 1-beta; nuclear corepressor KAP-1 sequence | | | | | | | | |
| GA_23615 | | | | 4 | 1 | 0 | 0 | 5 | es 12.01 | p = 0.02 |
| GA_23634 | | | | 4 | 1 | 0 | 0 | 5 | es 12.01 | p = 0.02 |
| GA_2367 | NM_003923 | forkhead box H1 (FOXH1) | [symbol] | 5 | 0 | 0 | 0 | 5 | es > 4 | p = 0.00 |
| GA_23673 | | | | 5 | 1 | 0 | 0 | 6 | es 15.02 | p = 0.00 |
| GA_23683 | | | | 4 | 1 | 1 | 0 | 6 | es 6.01 | p = 0.04 |
| GA_23981 | AK057602 | cDNA FLJ33040 fis, clone THYMU2000382, weakly | | 4 | 0 | 0 | 0 | 4 | es > 4 | p = 0.00 |
| | | similar to 60S RIBOSOMAL PROTEIN L12 | | | | | | | | |
| GA_2418 | NM_004317 | arsA arsenite transporter, ATP-binding, homolog 1 | | 6 | 3 | 1 | 1 | 11 | es 3.60 | p = 0.03 |
| | | (bacterial) (ASNA1) | | | | | | | | |
| GA_2436 | NM_004329 | bone morphogenetic protein receptor, type la | | 7 | 3 | 1 | 1 | 12 | es 4.20 | p = 0.01 |
| | | (BMPR1A) | | | | | | | | |
| GA_2442 | NM_004335 | bone marrow stromal cell antigen 2 (BST2) | | 13 | 0 | 2 | 3 | 18 | es 7.81 | p = 0.00 |
| GA_2443 | NM_004336 | BUB1 budding uninhibited by benzimidazoles 1 | | 10 | 5 | 4 | 2 | 21 | es 2.73 | p = 0.02 |
| | | homolog (yeast) (BUB1) | | | | | | | | |
| GA_2444 | NM_004725 | BUB3 budding uninhibited by benzimidazoles 3 | | 12 | 4 | 7 | 4 | 27 | es 2.40 | p = 0.03 |
| | | homolog (yeast) (BUB3) | | | | | | | | |
| GA_2447 | NM_004341 | carbamoyl-phosphate synthetase 2, aspartate | | 11 | 8 | 2 | 1 | 22 | es 3.00 | p = 0.01 |
| | | transcarbamylase, and dihydroorotase (CAD), | | | | | | | | |
| | | nuclear gene encoding mitochondrial protein | | | | | | | | |
| GA_2467 | NM_004804 | WD40 protein Ciao1 (CIAO1) | | 8 | 0 | 1 | 2 | 11 | es 8.01 | p = 0.00 |
| GA_2496 | NM_004229 | cofactor required for Sp1 transcriptional activation, | [symbol] | 7 | 1 | 1 | 2 | 11 | es 5.26 | p = 0.01 |
| | | subunit 2, 150 kDa (CRSP2) | | | | | | | | |
| GA_2501 | NM_080598 | HLA-B associated transcript 1 (BAT1), transcript | | 24 | 13 | 13 | 9 | 59 | es 2.06 | p = 0.01 |
| | | variant 2 | | | | | | | | |
| GA_2621 | NM_004135 | isocitrate dehydrogenase 3 (NAD+) gamma (IDH3G) | | 5 | 2 | 0 | 1 | 8 | es 5.01 | p = 0.03 |
| GA_2641 | NM_017522 | low density lipoprotein receptor-related protein 8, | | 7 | 0 | 0 | 2 | 9 | es 10.51 | p = 0.00 |
| | | apolipoprotein e receptor (LRP8), transcript variant 3 | | | | | | | | |
| GA_2643 | NM_004635 | mitogen-activated protein kinase-activated protein | | 6 | 0 | 1 | 2 | 9 | es 6.01 | p = 0.01 |
| | | kinase 3 (MAPKAPK3) | | | | | | | | |
| GA_2644 | NM_004526 | MCM2 minichromosome maintenance deficient 2, | | 23 | 8 | 6 | 4 | 41 | es 3.84 | p = 0.00 |
| | | mitotin (S. cerevisiae) (MCM2) | | | | | | | | |
| GA_2717 | NM_004703 | rabaptin-5 (RAB5EP) | | 5 | 1 | 1 | 0 | 7 | es 7.51 | p = 0.01 |
| GA_2728 | NM_004168 | succinate dehydrogenase complex, subunit A, | | 5 | 2 | 0 | 2 | 9 | es 3.75 | p = 0.05 |
| | | flavoprotein (Fp) (SDHA), nuclear gene encoding | | | | | | | | |
| | | mitochondrial protein | | | | | | | | |
| GA_2751 | NM_004596 | small nuclear ribonucleoprotein polypeptide A | | 11 | 3 | 4 | 5 | 23 | es 2.75 | p = 0.02 |
| | | (SNRPA) | | | | | | | | |
| GA_2762 | NM_004819 | symplekin; Huntingtin interacting protein I (SPK) | | 10 | 5 | 6 | 1 | 22 | es 2.50 | p = 0.04 |
| GA_2784 | NM_004818 | prp28, U5 snRNP 100 kd protein (U5-100 K) | | 16 | 14 | 3 | 3 | 36 | es 2.40 | p = 0.01 |
| GA_2791 | NM_004652 | ubiquitin specific protease 9, X chromosome (fat | | 10 | 2 | 2 | 1 | 15 | es 6.01 | p = 0.00 |
| | | facets-like Drosophila) (USP9X), transcript variant 1 | | | | | | | | |
| GA_2800 | NM_004629 | Fanconi anemia, complementation group G | | 5 | 0 | 2 | 1 | 8 | es 5.01 | p = 0.03 |
| | | (FANCG) | | | | | | | | |
| GA_2840 | NM_004960 | fusion, derived from t(12; 16) malignant liposarcoma | | 14 | 2 | 4 | 1 | 21 | es 6.01 | p = 0.00 |
| | | (FUS) | | | | | | | | |
| GA_2857 | NM_004987 | LIM and senescent cell antigen-like domains 1 | | 5 | 2 | 0 | 1 | 8 | es 5.01 | p = 0.03 |
| | | (LIMS1) | | | | | | | | |
| GA_2868 | NM_005006 | NADH dehydrogenase (ubiquinone) Fe-S protein 1, | | 6 | 1 | 2 | 2 | 11 | es 3.60 | p = 0.03 |
| | | 75 kDa (NADH-coenzyme Q reductase) (NDUFS1) | | | | | | | | |
| GA_2889 | NM_005032 | plastin 3 (T isoform) (PLS3) | | 35 | 18 | 7 | 19 | 79 | es 2.39 | p = 0.00 |
| GA_2897 | NM_005044 | protein kinase, X-linked (PRKX) | | 6 | 3 | 0 | 1 | 10 | es 4.51 | p = 0.02 |
| GA_2898 | NM_005049 | PWP2 periodic tryptophan protein homolog (yeast) | | 6 | 0 | 1 | 2 | 9 | es 6.01 | p = 0.01 |
| | | (PWP2H) | | | | | | | | |
| GA_2937 | NM_005207 | v-crk sarcoma virus CT10 oncogene homolog | | 6 | 1 | 0 | 0 | 7 | es 18.02 | p = 0.00 |
| | | (avian)-like (CRKL) | | | | | | | | |
| GA_2945 | NM_005232 | EphA1 (EPHA1) | | 5 | 1 | 1 | 1 | 8 | es 5.01 | p = 0.03 |
| GA_2962 | NM_005314 | gastrin-releasing peptide receptor (GRPR) | | 4 | 0 | 0 | 0 | 4 | es > 4 | p = 0.00 |
| GA_2984 | NM_005474 | histone deacetylase 5 (HDAC5), transcript variant 1 | | 6 | 4 | 1 | 0 | 11 | es 3.60 | p = 0.03 |
| GA_2988 | NM_005397 | podocalyxin-like (PODXL) | | 59 | 23 | 5 | 8 | 95 | es 4.92 | p = 0.00 |
| GA_3017 | NM_000098 | carnitine palmitoyltransferase II (CPT2), nuclear | | 4 | 1 | 1 | 0 | 6 | es 6.01 | p = 0.04 |
| | | gene encoding mitochondrial protein | | | | | | | | |
| GA_3024 | NM_003902 | far upstream element (FUSE) binding protein 1 | [symbol] | 13 | 4 | 6 | 3 | 26 | es 3.00 | p = 0.01 |
| | | (FUBP1) | | | | | | | | |
| GA_3042 | NM_005760 | CCAAT-box-binding transcription factor (CBF2) | [symbol] | 9 | 2 | 2 | 3 | 16 | es 3.86 | p = 0.01 |
| GA_3055 | NM_005864 | signal transduction protein (SH3 containing) (EFS2), | | 6 | 1 | 0 | 1 | 8 | es 9.01 | p = 0.00 |
| | | transcript variant 1 | | | | | | | | |
| GA_3112 | NM_005789 | proteasome (prosome, macropain) activator subunit | | 12 | 2 | 6 | 2 | 22 | es 3.60 | p = 0.00 |
| | | 3 (PA28 gamma; Ki) (PSME3) | | | | | | | | |
| GA_3118 | NM_005778 | RNA binding motif protein 5 (RBM5) | | 11 | 6 | 4 | 4 | 25 | es 2.36 | p = 0.04 |
| GA_3130 | NM_005785 | hypothetical SBBI03 protein (SBB103) | | 4 | 1 | 0 | 0 | 5 | es 12.01 | p = 0.02 |
| GA_3134 | NM_005877 | splicing factor 3a, subunit 1, 120 kDa (SF3A1) | | 10 | 1 | 4 | 3 | 18 | es 3.75 | p = 0.01 |
| GA_3137 | NM_005628 | solute carrier family 1 (neutral amino acid | | 23 | 11 | 2 | 13 | 49 | es 2.66 | p = 0.00 |
| | | transporter), member 5 (SLC1A5) | | | | | | | | |
| GA_3144 | NM_005839 | serine/arginine repetitive matrix 1 (SRRM1) | | 16 | 6 | 5 | 8 | 35 | es 2.53 | p = 0.01 |
| GA_3150 | NM_139315 | TAF6 RNA polymerase II, TATA box binding protein | | 4 | 0 | 0 | 0 | 4 | es > 4 | p = 0.00 |
| | | (TBP)-associated factor, 80 kDa (TAF6), transcript | | | | | | | | |
| | | variant 2 | | | | | | | | |
| GA_3175 | NM_005741 | zinc finger protein 263 (ZNF263) | [symbol] | 7 | 4 | 0 | 1 | 12 | es 4.20 | p = 0.01 |
| GA_3178 | NM_006017 | prominin-like 1 (mouse) (PROML1) | | 7 | 2 | 2 | 0 | 11 | es 5.26 | p = 0.01 |
| GA_3183 | NM_006035 | CDC42 binding protein kinase beta (DMPK-like) | | 13 | 5 | 0 | 3 | 21 | es 4.88 | p = 0.00 |
| | | (CDC42BPB) | | | | | | | | |
| GA_3219 | NM_005928 | milk fat globule-EGF factor 8 protein (MFGE8) | | 30 | 11 | 11 | 14 | 66 | es 2.50 | p = 0.00 |
| GA_32806 | BE568403 | 601341979F1 NIH_MGC_53cDNA clone | | 9 | 2 | 5 | 2 | 18 | es 3.00 | p = 0.02 |
| | | IMAGE: 3684283 5′ sequence | | | | | | | | |
| GA_32836 | AK055259 | cDNA FLJ30697 fis, clone FCBBF2000815, weakly | | 4 | 0 | 1 | 1 | 6 | es 6.01 | p = 0.04 |
| | | similar to ZYXIN | | | | | | | | |
| GA_32842 | | | | 8 | 3 | 0 | 0 | 11 | es 8.01 | p = 0.00 |
| GA_32860 | | | | 7 | 0 | 0 | 0 | 7 | es > 4 | p = 0.00 |
| GA_32868 | AK091598 | cDNA FLJ34279 fis, clone FEBRA2003833 | | 4 | 0 | 0 | 0 | 4 | es > 4 | p = 0.00 |
| | | sequence | | | | | | | | |
| GA_32887 | NM_006141 | dynein, cytoplasmic, light intermediate polypeptide 2 | | 7 | 2 | 0 | 2 | 11 | es 5.26 | p = 0.01 |
| | | (DNCLI2) | | | | | | | | |
| GA_32895 | | | | 5 | 4 | 0 | 0 | 9 | es 3.75 | p = 0.05 |
| GA_32908 | AL832758 | mRNA; cDNA DKFZp686C0927 (from clone | | 4 | 0 | 0 | 0 | 4 | es > 4 | p = 0.00 |
| | | DKFZp686C0927) sequence | | | | | | | | |
| GA_32913 | | | | 4 | 0 | 0 | 0 | 4 | es > 4 | p = 0.00 |
| GA_32917 | | | | 4 | 0 | 0 | 0 | 4 | es > 4 | p = 0.00 |
| GA_32926 | | | | 7 | 0 | 0 | 0 | 7 | es > 4 | p = 0.00 |
| GA_32947 | | | | 4 | 0 | 2 | 0 | 6 | es 6.01 | p = 0.04 |
| GA_32979 | | | | 4 | 0 | 0 | 0 | 4 | es > 4 | p = 0.00 |
| GA_32985 | | | | 4 | 0 | 0 | 0 | 4 | es > 4 | p = 0.00 |
| GA_3321 | NM_006345 | chromosome 4 open reading frame 1 (C4orf1) | | 10 | 5 | 4 | 2 | 21 | es 2.73 | p = 0.02 |
| GA_33423 | NM_002537 | ornithine decarboxylase antizyme 2 (OAZ2) | | 18 | 1 | 7 | 3 | 29 | es 4.91 | p = 0.00 |
| GA_3343 | NM_006392 | nucleolar protein 5A (56 kDa with KKE/D repeat) | | 16 | 5 | 11 | 5 | 37 | es 2.29 | p = 0.02 |
| | | (NOL5A) | | | | | | | | |
| GA_33455 | NM_006047 | RNA binding motif protein 12 (RBM12), transcript | | 17 | 4 | 3 | 4 | 28 | es 4.64 | p = 0.00 |
| | | variant 1 | | | | | | | | |
| GA_33475 | NM_004902 | RNA-binding region (RNP1, RRM) containing 2 | | 12 | 2 | 8 | 2 | 24 | es 3.00 | p = 0.01 |
| | | (RNPC2) | | | | | | | | |
| GA_33503 | NM_018135 | mitochondrial ribosomal protein S18A (MRPS18A), | | 4 | 1 | 1 | 0 | 6 | es 6.01 | p = 0.04 |
| | | nuclear gene encoding mitochondrial protein | | | | | | | | |
| GA_33528 | NM_032803 | solute carrier family 7 (cationic amino acid | | 4 | 0 | 1 | 0 | 5 | es 12.01 | p = 0.02 |
| | | transporter, y+ system), member 3 (SLC7A3) | | | | | | | | |
| GA_33533 | BC037428 | Unknown (protein for MGC: 46327) sequence | | 7 | 4 | 1 | 1 | 13 | es 3.50 | p = 0.02 |
| GA_33548 | NM_015638 | chromosome 20 open reading frame 188 | | 7 | 3 | 0 | 1 | 11 | es 5.26 | p = 0.01 |
| | | (C20orf188) | | | | | | | | |
| GA_33588 | AL832967 | mRNA; cDNA DKFZp666B082 (from clone | | 5 | 0 | 2 | 1 | 8 | es 5.01 | p = 0.03 |
| | | DKFZp666B082) sequence | | | | | | | | |
| GA_33680 | NM_016089 | KRAB-zinc finger protein SZF1-1 (SZF1) | [symbol] | 15 | 0 | 1 | 0 | 16 | es 45.05 | p = 0.00 |
| GA_33684 | NM_005186 | calpain 1, (mu/l) large subunit (CAPN1) | | 13 | 8 | 1 | 5 | 27 | es 2.79 | p = 0.01 |
| GA_33691 | AL117507 | mRNA; cDNA DKFZp434F1935 (from clone | | 4 | 1 | 1 | 0 | 6 | es 6.01 | p = 0.04 |
| | | DKFZp434F1935); partial cds | | | | | | | | |
| GA_33704 | AL833549 | mRNA; cDNA DKFZp686N183 (from clone | | 4 | 1 | 1 | 0 | 6 | es 6.01 | p = 0.04 |
| | | DKFZp686N183) sequence | | | | | | | | |
| GA_33730 | AL832779 | mRNA; cDNA DKFZp686H157 (from clone | | 4 | 0 | 1 | 1 | 6 | es 6.01 | p = 0.04 |
| | | DKFZp686H157) sequence | | | | | | | | |
| GA_33747 | NM_032737 | lamin B2 (LMNB2) | | 11 | 8 | 3 | 3 | 25 | es 2.36 | p = 0.04 |
| GA_33755 | NM_033547 | hypothetical gene MGC16733 similar to CG12113 | | 5 | 0 | 0 | 1 | 6 | es 15.02 | p = 0.00 |
| | | (MGC16733) | | | | | | | | |
| GA_33772 | BF223023 | 7q27f09.x1 NCI_CGAP_GC6cDNA clone | | 5 | 0 | 0 | 0 | 5 | es > 4 | p = 0.00 |
| | | IMAGE: 3699616 3′ sequence | | | | | | | | |
| GA_33816 | NM_015850 | fibroblast growth factor receptor 1 (fms-related | | 35 | 12 | 9 | 5 | 61 | es 4.04 | p = 0.00 |
| | | tyrosine kinase 2, Pfeiffer syndrome) (FGFR1), | | | | | | | | |
| | | transcript variant 2 | | | | | | | | |
| GA_33874 | NM_017730 | hypothetical protein FLJ20259 (FLJ20259) | | 19 | 6 | 4 | 4 | 33 | es 4.08 | p = 0.00 |
| GA_33876 | NM_148904 | oxysterol binding protein-like 9 (OSBPL9), transcript | | 5 | 1 | 0 | 2 | 8 | es 5.01 | p = 0.03 |
| | | variant 1 | | | | | | | | |
| GA_33877 | NM_020796 | sema domain, transmembrane domain (TM), and | | 16 | 1 | 11 | 4 | 32 | es 3.00 | p = 0.00 |
| | | cytoplasmic domain, (semaphorin) 6A (SEMA6A) | | | | | | | | |
| GA_33959 | NM_030964 | sprouty homolog 4 (Drosophila) (SPRY4) | | 4 | 1 | 0 | 0 | 5 | es 12.01 | p = 0.02 |
| GA_34010 | AK000089 | cDNA FLJ20082 fis, clone COL03245 | | 8 | 0 | 3 | 0 | 11 | es 8.01 | p = 0.00 |
| GA_34047 | NM_170752 | chromodomain protein, Y chromosome-like (CDYL), | | 8 | 1 | 1 | 1 | 11 | es 8.01 | p = 0.00 |
| | | transcript variant 3 | | | | | | | | |
| GA_34061 | NM_152429 | hypothetical protein MGC39320 (MGC39320) | | 7 | 1 | 0 | 1 | 9 | es 10.51 | p = 0.00 |
| GA_3407 | NM_006328 | RNA binding motif protein 14 (RBM14) | | 16 | 3 | 4 | 3 | 26 | es 4.81 | p = 0.00 |
| GA_34077 | NM_133457 | likely ortholog of mouse type XXVI collagen | | 7 | 0 | 4 | 2 | 13 | es 3.50 | p = 0.02 |
| | | (COL26A1) | | | | | | | | |
| GA_34137 | NM_020314 | esophageal cancer associated protein (MGC16824) | | 6 | 1 | 0 | 0 | 7 | es 18.02 | p = 0.00 |
| GA_34200 | NM_005763 | aminoadipate-semialdehyde synthase (AASS) | | 10 | 0 | 0 | 2 | 12 | es 15.02 | p = 0.00 |
| GA_34219 | NM_018449 | ubiquitin associated protein 2 (UBAP2), transcript | | 6 | 2 | 1 | 0 | 9 | es 6.01 | p = 0.01 |
| | | variant 1 | | | | | | | | |
| GA_34245 | NM_004922 | SEC24 related gene family, member C (S. | | 10 | 6 | 0 | 1 | 17 | es 4.29 | p = 0.00 |
| | | cerevisiae) (SEC24C) | | | | | | | | |
| GA_34270 | NM_152758 | hypothetical protein FLJ31657 (FLJ31657) | | 5 | 2 | 1 | 0 | 8 | es 5.01 | p = 0.03 |
| GA_34280 | NM_000702 | ATPase, Na+/K+ transporting, alpha 2 (+) | | 4 | 0 | 0 | 0 | 4 | es > 4 | p = 0.00 |
| | | polypeptide (ATP1A2) | | | | | | | | |
| GA_34320 | NM_006461 | sperm associated antigen 5 (SPAG5) | | 14 | 6 | 5 | 2 | 27 | es 3.23 | p = 0.00 |
| GA_34322 | NM_023926 | hypothetical protein FLJ12895 (FLJ12895) | | 5 | 0 | 1 | 2 | 8 | es 5.01 | p = 0.03 |
| GA_3436 | NM_018062 | hypothetical protein FLJ10335 (FLJ10335) | | 5 | 1 | 3 | 0 | 9 | es 3.75 | p = 0.05 |
| GA_34419 | NM_002952 | ribosomal protein S2 (RPS2) | | 19 | 5 | 11 | 7 | 42 | es 2.48 | p = 0.00 |
| GA_34438 | NM_006521 | transcription factor binding to IGHM enhancer 3 | [symbol] | 5 | 2 | 0 | 2 | 9 | es 3.75 | p = 0.05 |
| | | (TFE3) | | | | | | | | |
| GA_34480 | NM_012218 | interleukin enhancer binding factor 3, 90 kDa (ILF3), | [symbol] | 41 | 26 | 13 | 20 | 100 | es 2.09 | p = 0.00 |
| | | transcript variant 1 | | | | | | | | |
| GA_34503 | NM_005762 | tripartite motif-containing 28 (TRIM28) | | 13 | 6 | 8 | 2 | 29 | es 2.44 | p = 0.02 |
| GA_34505 | NM_002065 | glutamate-ammonia ligase (glutamine synthase) | | 21 | 1 | 8 | 2 | 32 | es 5.73 | p = 0.00 |
| | | (GLUL) | | | | | | | | |
| GA_34522 | NM_000071 | cystathionine-beta-synthase (CBS) | | 7 | 2 | 1 | 2 | 12 | es 4.20 | p = 0.01 |
| GA_34539 | NM_002880 | v-raf-1 murine leukemia viral oncogene homolog 1 | | 14 | 7 | 3 | 0 | 24 | es 4.20 | p = 0.00 |
| | | (RAF1) | | | | | | | | |
| GA_34563 | NM_007192 | suppressor of Ty 16 homolog (S. cerevisiae) | | 9 | 1 | 1 | 3 | 14 | es 5.41 | p = 0.00 |
| | | (SUPT16H) | | | | | | | | |
| GA34594 | NM_004426 | polyhomeotic-like 1 (Drosophila) (PHC1) | [symbol] | 6 | 0 | 0 | 0 | 6 | es > 4 | p = 0.00 |
| GA_34606 | NM_015570 | autism susceptibility candidate 2 (AUTS2) | | 7 | 0 | 0 | 2 | 9 | es 10.51 | p = 0.00 |
| GA_34626 | NM_004911 | protein disulfide isomerase related protein (calcium- | | 5 | 2 | 1 | 1 | 9 | es 3.75 | p = 0.05 |
| | | binding protein, intestinal-related) (ERP70) | | | | | | | | |
| GA_34655 | X74794 | P1 Cdc21 protein sequence | | 34 | 9 | 5 | 4 | 52 | es 5.67 | p = 0.00 |
| GA_34679 | NM_002015 | forkhead box O1A (rhabdomyosarcoma) (FOXO1A) | [symbol] | 4 | 0 | 1 | 1 | 6 | es 6.01 | p = 0.04 |
| GA_34715 | NM_002421 | matrix metalloproteinase 1 (interstitial collagenase) | | 5 | 1 | 0 | 2 | 8 | es 5.01 | p = 0.03 |
| | | (MMP1) | | | | | | | | |
| GA_34820 | NM_024656 | hypothetical protein FLJ22329 (FLJ22329) | | 5 | 1 | 1 | 1 | 8 | es 5.01 | p = 0.03 |
| GA_34875 | NM_004459 | fetal Alzheimer antigen (FALZ) | | 5 | 2 | 0 | 2 | 9 | es 3.75 | p = 0.05 |
| GA_35037 | NM_004426 | polyhomeotic-like 1 (Drosophila) (PHC1) | [symbol] | 34 | 3 | 2 | 5 | 44 | es 10.21 | p = 0.00 |
| GA_35125 | NM_005386 | neuronatin (NNAT) | | 5 | 3 | 0 | 1 | 9 | es 3.75 | p = 0.05 |
| GA_35141 | NM_018555 | zinc finger protein 331; zinc finger protein 463 | [symbol] | 13 | 2 | 5 | 2 | 22 | es 4.34 | p = 0.00 |
| | | (ZNF361) | | | | | | | | |
| GA_35150 | AB014542 | KIAA0642 protein sequence | | 5 | 1 | 2 | 1 | 9 | es 3.75 | p = 0.05 |
| GA_35158 | NM_015327 | KIAA1089 protein (KIAA1089) | | 10 | 6 | 2 | 2 | 20 | es 3.00 | p = 0.02 |
| GA_3520 | NM_005915 | MCM6 minichromosome maintenance deficient 6 | | 12 | 5 | 5 | 2 | 24 | es 3.00 | p = 0.01 |
| | | (MIS5 homolog, S. pombe) (S. cerevisiae) (MCM6) | | | | | | | | |
| GA_35206 | NM_005678 | SNRPN upstream reading frame (SNURF), | | 20 | 10 | 9 | 9 | 48 | es 2.15 | p = 0.01 |
| | | transcript variant 1 | | | | | | | | |
| GA_35221 | NM_020442 | KIAA1885 protein (DKFZP434L1435) | | 6 | 0 | 0 | 0 | 6 | es > 4 | p = 0.00 |
| GA_35231 | NM_014389 | proline and glutamic acid rich nuclear protein | | 14 | 11 | 3 | 1 | 29 | es 2.80 | p = 0.01 |
| | | (PELP1) | | | | | | | | |
| GA_35233 | NM_138615 | DEAD/H (Asp-Glu-Ala-Asp/His) box polypeptide 30 | | 11 | 3 | 4 | 5 | 23 | es 2.75 | p = 0.02 |
| | | (DDX30), transcript variant 1 | | | | | | | | |
| GA_35239 | NM_014633 | KIAA0155 gene product (KIAA0155) | | 5 | 1 | 2 | 0 | 8 | es 5.01 | p = 0.03 |
| GA_35260 | NM_004104 | fatty acid synthase (FASN) | | 6 | 2 | 0 | 1 | 9 | es 6.01 | p = 0.01 |
| GA_35393 | NM_006861 | RAB35, member RAS oncogene family (RAB35) | | 7 | 2 | 2 | 1 | 12 | es 4.20 | p = 0.01 |
| GA_35395 | NM_024662 | hypothetical protein FLJ10774 (FLJ10774) | | 6 | 4 | 0 | 1 | 11 | es 3.60 | p = 0.03 |
| GA_35405 | | | | 12 | 8 | 3 | 1 | 24 | es 3.00 | p = 0.01 |
| GA_35422 | NM_021211 | transposon-derived Buster1 transposase-like protein | | 4 | 0 | 0 | 2 | 6 | es 6.01 | p = 0.04 |
| | | (LOC58486) | | | | | | | | |
| GA_35457 | AJ459424 | JEMMA protein sequence | | 7 | 1 | 2 | 1 | 11 | es 5.26 | p = 0.01 |
| GA_35481 | NM_006452 | phosphoribosylaminoimidazole carboxylase, | | 36 | 14 | 13 | 9 | 72 | es 3.00 | p = 0.00 |
| | | phosphoribosylaminoimidazole succinocarboxamide | | | | | | | | |
| | | synthetase (PAICS) | | | | | | | | |
| GA_35495 | NM_003472 | DEK oncogene (DNA binding) (DEK) | [symbol] | 16 | 3 | 8 | 10 | 37 | es 2.29 | p = 0.02 |
| GA_35547 | NM_032202 | hypothetical protein KIAA1109 (KIAA1109) | | 4 | 0 | 0 | 2 | 6 | es 6.01 | p = 0.04 |
| GA_35558 | AL831917 | hypothetical protein sequence | | 6 | 1 | 1 | 1 | 9 | es 6.01 | p = 0.01 |
| GA_3559 | NM_005629 | solute carrier family 6 (neurotransmitter transporter, | | 5 | 1 | 0 | 1 | 7 | es 7.51 | p = 0.01 |
| | | creatine), member 8 (SLC6A8) | | | | | | | | |
| GA_35606 | NM_024586 | oxysterol binding protein-like 9 (OSBPL9), transcript | | 4 | 1 | 1 | 0 | 6 | es 6.01 | p = 0.04 |
| | | variant 6 | | | | | | | | |
| GA_35607 | AB002366 | KIAA0368 sequence | | 8 | 4 | 2 | 3 | 17 | es 2.67 | p = 0.05 |
| GA_35615 | NM_000251 | mutS homolog 2, colon cancer, nonpolyposis type 1 | | 16 | 6 | 6 | 0 | 28 | es 4.00 | p = 0.00 |
| | | (E. coli) (MSH2) | | | | | | | | |
| GA_35687 | NM_033502 | transcriptional regulating protein 132 (TReP-132), | [symbol] | 5 | 0 | 0 | 0 | 5 | es > 4 | p = 0.00 |
| | | transcript variant 1 | | | | | | | | |
| GA_35693 | NM_014782 | armadillo repeat protein ALEX2 (ALEX2) | [symbol] | 12 | 8 | 4 | 3 | 27 | es 2.40 | p = 0.03 |
| GA_35762 | NM_020765 | retinoblastoma-associated factor 600 (RBAF600) | | 12 | 4 | 3 | 1 | 20 | es 4.51 | p = 0.00 |
| GA_35833 | NM_015878 | ornithine decarboxylase antizyme inhibitor (OAZIN), | | 17 | 8 | 10 | 6 | 41 | es 2.13 | p = 0.02 |
| | | transcript variant 1 | | | | | | | | |
| GA_35852 | AK056479 | cDNA FLJ31917 fis, clone NT2RP7004925, weakly | | 4 | 2 | 0 | 0 | 6 | es 6.01 | p = 0.04 |
| | | similar to VASODILATOR-STIMULATED | | | | | | | | |
| | | PHOSPHOPROTEIN | | | | | | | | |
| GA_35869 | AB011112 | KIAA0540 protein sequence | | 5 | 2 | 1 | 0 | 8 | es 5.01 | p = 0.03 |
| GA_35905 | NM_006640 | MLL septin-like fusion (MSF) | | 28 | 25 | 6 | 6 | 65 | es 2.27 | p = 0.00 |
| GA_35913 | NM_018265 | hypothetical protein FLJ10901 (FLJ10901) | | 5 | 0 | 1 | 1 | 7 | es 7.51 | p = 0.01 |
| GA_3593 | NM_000270 | nucleoside phosphorylase (NP) | | 5 | 1 | 1 | 1 | 8 | es 5.01 | p = 0.03 |
| GA_35955 | NM_022754 | sideroflexin 1 (SFXN1) | | 5 | 1 | 1 | 0 | 7 | es 7.51 | p = 0.01 |
| Gk_35984 | NM_015340 | leucyl-tRNA synthetase, mitochondrial (LARS2), | | 4 | 0 | 2 | 0 | 6 | es 6.01 | p = 0.04 |
| | | nuclear gene encoding mitochondrial protein | | | | | | | | |
| GA_36015 | NM_015341 | barren homolog (Drosophila) (BRRN1) | | 9 | 1 | 1 | 2 | 13 | es 6.76 | p = 0.00 |
| GA_36017 | AK074137 | FLJ00210 protein sequence | | 4 | 0 | 1 | 0 | 5 | es 12.01 | p = 0.02 |
| GA_36019 | NM_012426 | splicing factor 3b, subunit 3, 130 kDa (SF3B3) | | 11 | 3 | 2 | 3 | 19 | es 4.13 | p = 0.00 |
| GA_36080 | NM_152333 | chromosome 14 open reading frame 69 (C14orf69) | | 14 | 1 | 1 | 3 | 19 | es 8.41 | p = 0.00 |
| GA_36090 | NM_020444 | KIAA1191 protein (KIAA1191) | | 9 | 7 | 1 | 2 | 19 | es 2.70 | p = 0.03 |
| GA_3611 | NM_001211 | BUB1 budding uninhibited by benzimidazoles 1 | | 13 | 4 | 4 | 4 | 25 | es 3.25 | p = 0.00 |
| | | homolog beta (yeast) (BUB1B) | | | | | | | | |
| GA_36126 | NM_004286 | GTP binding protein 1 (GTPBP1) | | 4 | 1 | 0 | 0 | 5 | es 12.01 | p = 0.02 |
| GA_36127 | NM_016121 | NY-REN-45 antigen (NY-REN-45) | | 5 | 1 | 2 | 1 | 9 | es 3.75 | p = 0.05 |
| GA_36129 | NM_018353 | hypothetical protein FLJ11186 (FLJ11186) | | 10 | 0 | 3 | 3 | 16 | es 5.01 | p = 0.00 |
| GA_36133 | NM_020428 | CTL2 gene (CTL2) | | 9 | 6 | 0 | 0 | 15 | es 4.51 | p = 0.00 |
| GA_36137 | NM_007363 | non-POU domain containing, octamer-binding | [symbol] | 39 | 12 | 22 | 14 | 87 | es 2.44 | p = 0.00 |
| | | (NONO) | | | | | | | | |
| GA_36139 | NM_004990 | methionine-tRNA synthetase (MARS) | | 11 | 3 | 1 | 0 | 15 | es 8.26 | p = 0.00 |
| GA_36155 | AB020719 | KIAA0912 protein sequence | | 5 | 1 | 1 | 0 | 7 | es 7.51 | p = 0.01 |
| GA_36183 | NM_016333 | serine/arginine repetitive matrix 2 (SRRM2) | | 23 | 21 | 9 | 1 | 54 | es 2.23 | p = 0.00 |
| GA_36184 | NM_020151 | START domain containing 7 (STARD7), transcript | | 17 | 6 | 0 | 1 | 24 | es 7.29 | p = 0.00 |
| | | variant 1 | | | | | | | | |
| GA_36219 | NM_152392 | hypothetical protein DKFZp564C236 | | 7 | 1 | 2 | 1 | 11 | es 5.26 | p = 0.01 |
| | | (DKFZp564C236) | | | | | | | | |
| GA_36221 | NM_000966 | retinoic acid receptor, gamma (RARG) | [symbol] | 6 | 2 | 0 | 2 | 10 | es 4.51 | p = 0.02 |
| GA_36241 | NM_018031 | WD repeat domain 6 (WDR6), transcript variant 1 | | 29 | 20 | 11 | 7 | 67 | es 2.29 | p = 0.00 |
| GA_36270 | NM_003715 | vesicle docking protein p115 (VDP) | | 12 | 5 | 4 | 2 | 23 | es 3.28 | p = 0.01 |
| GA_3628 | NM_006579 | emopamil binding protein (sterol isomerase) (EBP) | | 7 | 1 | 3 | 0 | 11 | es 5.26 | p = 0.01 |
| GA_36307 | NM_015897 | protein inhibitor of activated STAT protein PIASy | | 5 | 2 | 2 | 0 | 9 | es 3.75 | p = 0.05 |
| | | (PIASY) | | | | | | | | |
| GA_36389 | NM_025256 | HLA-B associated transcript 8 (BAT8), transcript | | 11 | 5 | 6 | 2 | 24 | es 2.54 | p = 0.03 |
| | | variant NG36/G9a-SPI | | | | | | | | |
| GA_36450 | NM_003051 | solute carrier family 16 (monocarboxylic acid | | 22 | 7 | 7 | 5 | 41 | es 3.48 | p = 0.00 |
| | | transporters), member 1 (SLC16A1) | | | | | | | | |
| GA_36474 | X87832 | NOV | | 5 | 4 | 0 | 0 | 9 | es 3.75 | p = 0.05 |
| GA_36491 | NM_024611 | similar to NMDA receptor-regulated gene 2 (mouse) | | 6 | 4 | 0 | 1 | 11 | es 3.60 | p = 0.03 |
| | | (FLJ11896) | | | | | | | | |
| GA_36526 | NM_033557 | similar to putative transmembrane protein; homolog | | 6 | 3 | 2 | 0 | 11 | es 3.60 | p = 0.03 |
| | | of yeast Golgi membrane protein Yif1p (Yip1p- | | | | | | | | |
| | | interacting factor) (LOC90522) | | | | | | | | |
| GA_36545 | AB014600 | KIAA0700 protein sequence | | 8 | 4 | 1 | 3 | 16 | es 3.00 | p = 0.04 |
| GA_36581 | NM_018071 | hypothetical protein FLJ10357 (FLJ10357) | | 6 | 3 | 0 | 0 | 9 | es 6.01 | p = 0.01 |
| GA_36592 | AB002363 | KIAA0365 sequence | | 6 | 1 | 0 | 1 | 8 | es 9.01 | p = 0.00 |
| GA_36595 | NM_024718 | hypothetical protein FLJ10101 (FLJ10101) | | 8 | 4 | 2 | 3 | 17 | es 2.67 | p = 0.05 |
| GA_36643 | NM_003918 | glycogenin 2 (GYG2) | | 5 | 1 | 0 | 0 | 6 | es 15.02 | p = 0.00 |
| GA_36675 | NM_003605 | O-linked N-acetylglucosamine (GIcNAc) transferase | | 9 | 4 | 0 | 1 | 14 | es 5.41 | p = 0.00 |
| | | (UDP-N-acetylglucosamine: polypeptide-N- | | | | | | | | |
| | | acetylglucosaminyl transferase) (OGT) | | | | | | | | |
| GA_36692 | NM_015902 | progestin induced protein (DD5) | | 8 | 4 | 1 | 2 | 15 | es 3.43 | p = 0.02 |
| GA_36707 | NM_021627 | sentrin-specific protease (SENP2) | | 4 | 0 | 1 | 0 | 5 | es 12.01 | p = 0.02 |
| GA_36730 | AF164609 | endogenous retrovirus HERV-K101, complete | | 5 | 0 | 0 | 0 | 5 | es > 4 | p = 0.00 |
| | | sequence | | | | | | | | |
| GA_36734 | AF376802 | neuroligin 2 sequence | | 6 | 3 | 0 | 0 | 9 | es 6.01 | p = 0.01 |
| GA_36771 | NM_016238 | anaphase-promoting complex subunit 7 (ANAPC7) | | 6 | 0 | 1 | 0 | 7 | es 18.02 | p = 0.00 |
| GA_36788 | NM_000141 | fibroblast growth factor receptor 2 (bacteria- | | 9 | 5 | 1 | 2 | 17 | es 3.38 | p = 0.02 |
| | | expressed kinase, keratinocyte growth factor | | | | | | | | |
| | | receptor, craniofacial dysostosis 1, Crouzon | | | | | | | | |
| | | syndrome, Pfeiffer syndrome, Jackson-Weiss | | | | | | | | |
| | | syndrome) (FGFR2), transcript variant 1 | | | | | | | | |
| GA_36798 | NM_000071 | cystathionine-beta-synthase (CBS) | | 11 | 0 | 1 | 2 | 14 | es 11.01 | p = 0.00 |
| GA_36842 | NM_006197 | pericentriolar material 1 (PCM1) | | 6 | 3 | 1 | 1 | 11 | es 3.60 | p = 0.03 |
| GA_36897 | NM_006773 | DEAD/H (Asp-Glu-Ala-Asp/His) box polypeptide 18 | | 7 | 3 | 2 | 1 | 13 | es 3.50 | p = 0.02 |
| | | (Myc-regulated) (DDX18) | | | | | | | | |
| GA_36933 | NM_016424 | cisplatin resistance-associated overexpressed | | 19 | 1 | 4 | 7 | 31 | es 4.76 | p = 0.00 |
| | | protein (LUC7A) | | | | | | | | |
| GA_36936 | NM_149379 | Williams Beuren syndrome chromosome region 20C | | 11 | 6 | 4 | 1 | 22 | es 3.00 | p = 0.01 |
| | | (WBSCR20C), transcript variant 4 | | | | | | | | |
| GA_36951 | NM_005916 | MCM7 minichromosome maintenance deficient 7 (S. | | 19 | 3 | 6 | 11 | 39 | es 2.85 | p = 0.00 |
| | | cerevisiae) (MCM7) | | | | | | | | |
| GA_36957 | NM_024642 | UDP-N-acetyl-alpha-D-galactosamine: polypeptide | | 4 | 0 | 1 | 1 | 6 | es 6.01 | p = 0.04 |
| | | N-acetylgalactosaminyltransferase 12 (GalNAc-T12) | | | | | | | | |
| | | (GALNT12) | | | | | | | | |
| GA_36964 | NG_001332 | T cell receptor alpha delta locus (TCRA/TCRD) on | | 16 | 2 | 0 | 0 | 18 | es 24.03 | p = 0.00 |
| | | chromosome 14 | | | | | | | | |
| GA_36974 | AL834155 | mRNA; cDNA DKFZp761O0611 (from clone | | 4 | 1 | 0 | 1 | 6 | es 6.01 | p = 0.04 |
| | | DKFZp761O0611) sequence | | | | | | | | |
| GA_36977 | NM_020927 | KIAA1576 protein (KIAA1576) | | 9 | 2 | 1 | 0 | 12 | es 9.01 | p = 0.00 |
| GA_37071 | NM_153759 | DNA (cytosine-5-)-methyltransferase 3 alpha | | 9 | 2 | 1 | 1 | 13 | es 6.76 | p = 0.00 |
| | | (DNMT3A), transcript variant 2 | | | | | | | | |
| GA_37078 | NM_014977 | apoptotic chromatin condensation inducer in the | | 10 | 6 | 2 | 2 | 20 | es 3.00 | p = 0.02 |
| | | nucleus (ACINUS) | | | | | | | | |
| GA_37079 | NM_032156 | EEG1 (EEG1), transcript variant S | | 7 | 0 | 0 | 0 | 7 | es > 4 | p = 0.00 |
| GA_37094 | AL832758 | mRNA; cDNA DKFZp686C0927 (from clone | | 11 | 1 | 3 | 3 | 18 | es 4.72 | p = 0.00 |
| | | DKFZp686C0927) sequence | | | | | | | | |
| GA_37215 | NM_019023 | hypothetical protein FLJ10640 (FLJ10640) | | 7 | 1 | 3 | 0 | 11 | es 5.26 | p = 0.01 |
| GA_3723 | NM_003750 | eukaryotic translation initiation factor 3, subunit 10 | | 30 | 15 | 6 | 17 | 68 | es 2.37 | p = 0.00 |
| | | theta, 150/170 kDa (EIF3S10) | | | | | | | | |
| GA_37251 | NM_000604 | fibroblast growth factor receptor 1 (fms-related | | 7 | 1 | 5 | 0 | 13 | es 3.50 | p = 0.02 |
| | | tyrosine kinase 2, Pfeiffer syndrome) (FGFR1), | | | | | | | | |
| | | transcript variant 1 | | | | | | | | |
| GA_3730 | NM_003751 | eukaryotic translation initiation factor 3, subunit 9 | | 13 | 5 | 2 | 3 | 23 | es 3.90 | p = 0.00 |
| | | eta, 116 kDa (EIF3S9) | | | | | | | | |
| GA_37314 | NM_003169 | suppressor of Ty 5 homolog (S. cerevisiae) | | 14 | 6 | 1 | 1 | 22 | es 5.26 | p = 0.00 |
| | | (SUPT5H) | | | | | | | | |
| GA_37354 | NM_015726 | H326 (H326) | | 5 | 1 | 1 | 0 | 7 | es 7.51 | p = 0.01 |
| GA_37372 | NM_024658 | importin 4 (FLJ23338) | | 12 | 7 | 0 | 3 | 22 | es 3.60 | p = 0.00 |
| GA_37389 | NM_017647 | FtsJ homolog 3 (E. coli) (FTSJ3) | | 13 | 7 | 5 | 1 | 26 | es 3.00 | p = 0.01 |
| GA_37391 | NM_004938 | death-associated protein kinase 1 (DAPK1) | | 6 | 0 | 0 | 1 | 7 | es 18.02 | p = 0.00 |
| GA_37399 | NM_148842 | Williams-Beuren syndrome chromosome region 16 | | 10 | 0 | 1 | 2 | 13 | es 10.01 | p = 0.00 |
| | | (WBSCR16), transcript variant 2 | | | | | | | | |
| GA_37409 | NM_021145 | cyclin D binding myb-like transcription factor 1 | [symbol] | 5 | 1 | 0 | 2 | 8 | es 5.01 | p = 0.03 |
| | | (DMTF1) | | | | | | | | |
| GA_37424 | NM_152742 | hypothetical protein DKFZp547M109 | | 6 | 0 | 1 | 2 | 9 | es 6.01 | p = 0.01 |
| | | (DKFZp547M109) | | | | | | | | |
| GA_37431 | NM_006034 | p53-induced protein (PIG11) | | 7 | 4 | 1 | 0 | 12 | es 4.20 | p = 0.01 |
| GA_37478 | NM_014670 | basic leucine zipper and W2 domains 1 (BZW1) | | 24 | 13 | 11 | 9 | 57 | es 2.18 | p = 0.01 |
| GA_37504 | NM_153613 | PISC domain containing hypothetical protein | | 5 | 1 | 0 | 3 | 9 | es 3.75 | p = 0.05 |
| | | (LOC254531) | | | | | | | | |
| GA_37536 | AK026970 | cDNA: FLJ23317 fis, clone HEP12062, highly similar | | 5 | 2 | 1 | 0 | 8 | es 5.01 | p = 0.03 |
| | | to AF008936syntaxin-16B mRNA | | | | | | | | |
| GA_37538 | NM_080797 | death associated transcription factor 1 (DATF1), | [symbol] | 6 | 0 | 1 | 0 | 7 | es 18.02 | p = 0.00 |
| | | transcript variant 3 | | | | | | | | |
| GA_37589 | AL834216 | hypothetical protein sequence | | 4 | 0 | 1 | 0 | 5 | es 12.01 | p = 0.02 |
| GA_37595 | NM_015062 | KIAA0595 protein (KIAA0595) | | 7 | 3 | 0 | 1 | 11 | es 5.26 | p = 0.01 |
| GA_37606 | NM_019012 | phosphoinositol 3-phosphate-binding protein-2 | | 4 | 2 | 0 | 0 | 6 | es 6.01 | p = 0.04 |
| | | (PEPP2) | | | | | | | | |
| GA_37707 | NM_022574 | PERQ amino acid rich, with GYF domain 1 (PERQ1) | | 4 | 0 | 1 | 0 | 5 | es 12.01 | p = 0.02 |
| GA_37729 | NM_005436 | DNA segment on chromosome 10 (unique) 170 | | 8 | 4 | 1 | 3 | 16 | es 3.00 | p = 0.04 |
| | | (D10S170) | | | | | | | | |
| GA_37737 | NM_003707 | RuvB-like 1 (E. coli) (RUVBL1) | | 5 | 2 | 0 | 2 | 9 | es 3.75 | p = 0.05 |
| GA_37755 | NM_015044 | golgi associated, gamma adaptin ear containing, | | 13 | 5 | 0 | 2 | 20 | es 5.58 | p = 0.00 |
| | | ARF binding protein 2 (GGA2), transcript variant 1 | | | | | | | | |
| GA_37788 | NM_133631 | roundabout, axon guidance receptor, homolog 1 | | 7 | 4 | 1 | 0 | 12 | es 4.20 | p = 0.01 |
| | | (Drosophila) (ROBO1), transcript variant 2 | | | | | | | | |
| GA_37800 | NM_032701 | hypothetical protein MGC2705 (MGC2705) | | 4 | 1 | 0 | 1 | 6 | es 6.01 | p = 0.04 |
| GA_37805 | NM_025222 | hypothetical protein PRO2730 (PRO2730) | | 6 | 1 | 3 | 1 | 11 | es 3.60 | p = 0.03 |
| GA_37866 | NM_138927 | SON DNA binding protein (SON), transcript variant f | | 6 | 3 | 2 | 0 | 11 | es 3.60 | p = 0.03 |
| GA_37877 | NM_012215 | meningioma expressed antigen 5 (hyaluronidase) | | 10 | 4 | 3 | 3 | 20 | es 3.00 | p = 0.02 |
| | | (MGEA5) | | | | | | | | |
| GA_37884 | AB032993 | KIAA1167 protein sequence | | 5 | 2 | 1 | 0 | 8 | es 5.01 | p = 0.03 |
| GA_37904 | NM_000478 | alkaline phosphatase, liver/bone/kidney (ALPL) | | 4 | 1 | 1 | 0 | 6 | es 6.01 | p = 0.04 |
| GA_37914 | NM_153464 | interleukin enhancer binding factor 3, 90 kDa (ILF3), | [symbol] | 9 | 1 | 1 | 0 | 11 | es 13.52 | p = 0.00 |
| | | transcript variant 3 | | | | | | | | |
| GA_38001 | NM_152312 | hypothetical protein FLJ35207 (FLJ35207) | | 4 | 1 | 0 | 0 | 5 | es 12.01 | p = 0.02 |
| GA_38023 | NM_015846 | methyl-CpG binding domain protein 1 (MBD1), | | 7 | 0 | 1 | 0 | 8 | es 21.02 | p = 0.00 |
| | | transcript variant 1 | | | | | | | | |
| GA_38029 | | | | 4 | 1 | 0 | 0 | 5 | es 12.01 | p = 0.02 |
| GA_38084 | NM_015658 | DKFZP564C186 protein (DKFZP564C186) | | 13 | 5 | 3 | 5 | 26 | es 3.00 | p = 0.01 |
| GA_3818 | NM_006833 | COP9 subunit 6 (MOV34 homolog, 34 kD) (COPS6) | | 8 | 1 | 1 | 6 | 16 | es 3.00 | p = 0.04 |
| GA_38225 | NM_007152 | zinc finger protein 195 (ZNF195) | [symbol] | 4 | 0 | 2 | 0 | 6 | es 6.01 | p = 0.04 |
| GA_38238 | AL133439 | mRNA full length insert cDNA clone EUROIMAGE | | 4 | 0 | 2 | 0 | 6 | es 6.01 | p = 0.04 |
| | | 200978 | | | | | | | | |
| GA_38243 | BM920378 | AGENCOURT_6709352 NIH_MGC_122cDNA | | 5 | 2 | 1 | 1 | 9 | es 3.75 | p = 0.05 |
| | | clone IMAGE: 5750332 5′ sequence | | | | | | | | |
| GA_3826 | NM_006875 | pim-2 oncogene (PIM2) | | 5 | 0 | 1 | 0 | 6 | es 15.02 | p = 0.00 |
| GA_38266 | NM_144504 | junctional adhesion molecule 1 (JAM1), transcript | | 18 | 4 | 3 | 8 | 33 | es 3.60 | p = 0.00 |
| | | variant 5 | | | | | | | | |
| GA_38278 | NM_019852 | methyltransferase like 3 (METTL3) | | 8 | 0 | 4 | 3 | 15 | es 3.43 | p = 0.02 |
| GA_38283 | NM_013411 | adenylate kinase 2 (AK2), nuclear gene encoding | | 16 | 6 | 6 | 3 | 31 | es 3.20 | p = 0.00 |
| | | mitochondrial protein, transcript variant AK2B | | | | | | | | |
| GA_38292 | NM_005455 | zinc finger protein 265 (ZNF265) | [symbol] | 6 | 2 | 3 | 0 | 11 | es 3.60 | p = 0.03 |
| GA_38304 | NM_002394 | solute carrier family 3 (activators of dibasic and | | 4 | 0 | 1 | 0 | 5 | es 12.01 | p = 0.02 |
| | | neutral amino acid transport), member 2 (SLC3A2) | | | | | | | | |
| GA_38370 | NM_024923 | nucleoporin 210 (NUP210) | | 8 | 0 | 2 | 1 | 11 | es 8.01 | p = 0.00 |
| GA_38371 | NM_018003 | uveal autoantigen with coiled-coil domains and | | 5 | 1 | 1 | 2 | 9 | es 3.75 | p = 0.05 |
| | | ankyrin repeats (UACA) | | | | | | | | |
| GA_38377 | NM_033288 | KRAB zinc finger protein KR18 (KR18) | [symbol] | 5 | 2 | 1 | 0 | 8 | es 5.01 | p = 0.03 |
| GA_38426 | NG_001332 | T cell receptor alpha delta locus (TCRA/TCRD) on | | 7 | 1 | 2 | 0 | 10 | es 7.01 | p = 0.00 |
| | | chromosome 14 | | | | | | | | |
| GA_38431 | NM_021238 | TERA protein (TERA) | | 26 | 5 | 2 | 8 | 41 | es 5.21 | p = 0.00 |
| GA_38500 | AB040903 | KIAA1470 protein sequence | | 21 | 12 | 7 | 7 | 47 | es 2.43 | p = 0.00 |
| GA_3851 | NM_006759 | UDP-glucose pyrophosphorylase 2 (UGP2) | | 17 | 4 | 5 | 2 | 28 | es 4.64 | p = 0.00 |
| GA_38548 | AB033107 | KIAA1281 protein sequence | | 6 | 2 | 0 | 3 | 11 | es 3.60 | p = 0.03 |
| GA_3861 | NM_006845 | kinesin family member 2C (KIF2C) | | 9 | 1 | 4 | 1 | 15 | es 4.51 | p = 0.00 |
| GA_38627 | AL831836 | hypothetical protein sequence | | 5 | 1 | 1 | 2 | 9 | es 3.75 | p = 0.05 |
| GA_38635 | NM_133370 | KIAA1966 protein (KIAA1966) | | 9 | 4 | 4 | 2 | 19 | es 2.70 | p = 0.03 |
| GA_38666 | BC000401 | splicing factor 3b, subunit 2, 145 kD sequence | | 16 | 9 | 9 | 6 | 40 | es 2.00 | p = 0.04 |
| GA_38677 | NM_153280 | ubiquitin-activating enzyme E1 (A1S9T and BN75 | | 44 | 41 | 10 | 14 | 109 | es 2.03 | p = 0.00 |
| | | temperature sensitivity complementing) (UBE1), | | | | | | | | |
| | | transcript variant 2 | | | | | | | | |
| GA_38691 | NM_004550 | NADH dehydrogenase (ubiquinone) Fe-S protein 2, | | 9 | 1 | 2 | 6 | 18 | es 3.00 | p = 0.02 |
| | | 49 kDa (NADH-coenzyme Q reductase) (NDUFS2) | | | | | | | | |
| GA_387 | AB020648 | KIAA0841 protein sequence | | 4 | 1 | 1 | 0 | 6 | es 6.01 | p = 0.04 |
| GA_38786 | NM_138769 | mitochondrial Rho 2 (MIRO-2) | | 8 | 0 | 2 | 3 | 13 | es 4.81 | p = 0.01 |
| GA_38804 | NM_018249 | CDK5 regulatory subunit associated protein 2 | | 5 | 3 | 1 | 0 | 9 | es 3.75 | p = 0.05 |
| | | (CDK5RAP2) | | | | | | | | |
| GA_38826 | NM_133171 | engulfment and cell motility 2 (ced-12 homolog, C. | | 4 | 1 | 0 | 1 | 6 | es 6.01 | p = 0.04 |
| | | elegans) (ELMO2), transcript variant 1 | | | | | | | | |
| GA_38854 | NM_032228 | hypothetical protein FLJ22728 (FLJ22728) | | 5 | 2 | 0 | 2 | 9 | es 3.75 | p = 0.05 |
| GA_38867 | NM_018189 | hypothetical protein FLJ10713 (FLJ10713) | | 34 | 2 | 6 | 1 | 43 | es 11.35 | p = 0.00 |
| GA_3897 | NM_007015 | chondromodulin I precursor (CHM-I) | | 4 | 0 | 1 | 0 | 5 | es 12.01 | p = 0.02 |
| GA_3898 | NM_006892 | DNA (cytosine-5-)-methyltransferase 3 beta | | 49 | 2 | 3 | 1 | 55 | es 24.53 | p = 0.00 |
| | | (DNMT3B) | | | | | | | | |
| GA_3899 | NM_144733 | E1B-55 kDa-associated protein 5 (E1B-AP5), | | 23 | 16 | 6 | 7 | 52 | es 2.38 | p = 0.00 |
| | | transcript variant 2 | | | | | | | | |
| GA_3938 | NM_006925 | splicing factor, arginine/serine-rich 5 (SFRS5) | | 29 | 4 | 24 | 6 | 63 | es 2.56 | p = 0.00 |
| GA_3984 | NM_006114 | translocase of outer mitochondrial membrane 40 | | 7 | 1 | 2 | 2 | 12 | es 4.20 | p = 0.01 |
| | | homolog (yeast) (TOMM40) | | | | | | | | |
| GA_4038 | NM_007223 | putative G protein coupled receptor (GPR) | | 5 | 2 | 0 | 0 | 7 | es 7.51 | p = 0.01 |
| GA_4059 | NM_007221 | polyamine-modulated factor 1 (PMF1) | | 6 | 2 | 2 | 1 | 11 | es 3.60 | p = 0.03 |
| GA_4148 | NM_003826 | N-ethylmaleimide-sensitive factor attachment | | 4 | 1 | 0 | 1 | 6 | es 6.01 | p = 0.04 |
| | | protein, gamma (NAPG) | | | | | | | | |
| GA_4176 | NM_004448 | v-erb-b2 erythroblastic leukemia viral oncogene | | 15 | 11 | 2 | 5 | 33 | es 2.50 | p = 0.01 |
| | | homolog 2, neuro/glioblastoma derived oncogene | | | | | | | | |
| | | homolog (avian) (ERBB2) | | | | | | | | |
| GA_4247 | NM_001975 | enolase 2, (gamma, neuronal) (ENO2) | | 5 | 0 | 2 | 0 | 7 | es 7.51 | p = 0.01 |
| GA_4251 | NM_002528 | nth endonuclease III-like 1 (E. coli) (NTHL1) | | 4 | 0 | 0 | 1 | 5 | es 12.01 | p = 0.02 |
| GA_4253 | NM_004761 | RAB2, member RAS oncogene family-like (RAB2L) | | 6 | 3 | 2 | 0 | 11 | es 3.60 | p = 0.03 |
| GA_4255 | NM_006929 | superkiller viralicidic activity 2-like (S. cerevisiae) | | 5 | 4 | 0 | 0 | 9 | es 3.75 | p = 0.05 |
| | | (SKIV2L) | | | | | | | | |
| GA_4258 | NM_080911 | uracil-DNA glycosylase (UNG), nuclear gene | | 9 | 3 | 6 | 0 | 18 | es 3.00 | p = 0.02 |
| | | encoding mitochondrial protein, transcript variant 2 | | | | | | | | |
| GA_4263 | NM_006247 | protein phosphatase 5, catalytic subunit (PPP5C) | | 6 | 1 | 3 | 1 | 11 | es 3.60 | p = 0.03 |
| GA_4268 | NM_003852 | transcriptional intermediary factor 1 (TIF1) | [symbol] | 13 | 4 | 4 | 1 | 22 | es 4.34 | p = 0.00 |
| GA_4295 | NM_005255 | cyclin G associated kinase (GAK) | | 6 | 3 | 2 | 0 | 11 | es 3.60 | p = 0.03 |
| GA_4302 | NM_005054 | RAN binding protein 2-like 1 (RANBP2L1), transcript | | 4 | 0 | 0 | 1 | 5 | es 12.01 | p = 0.02 |
| | | variant 1 | | | | | | | | |
| GA_4332 | NM_019900 | ATP-binding cassette, sub-family C (CFTR/MRP), | | 8 | 3 | 2 | 1 | 14 | es 4.00 | p = 0.01 |
| | | member 1 (ABCC1), transcript variant 5 | | | | | | | | |
| GA_4446 | NM_002388 | MCM3 minichromosome maintenance deficient 3 (S. | | 38 | 4 | 8 | 7 | 57 | es 6.01 | p = 0.00 |
| | | cerevisiae) (MCM3) | | | | | | | | |
| GA_4478 | AK074826 | cDNA FLJ90345 fis, clone NT2RP2002974, highly | [symbol] | 4 | 0 | 0 | 0 | 4 | es > 4 | p = 0.00 |
| | | similar to HOMEOBOX PROTEIN SIX5 sequence | | | | | | | | |
| GA_4551 | NM_007375 | TAR DNA binding protein (TARDBP) | | 17 | 11 | 4 | 5 | 37 | es 2.55 | p = 0.01 |
| GA_4568 | NM_012100 | aspartyl aminopeptidase (DNPEP) | | 8 | 1 | 1 | 1 | 11 | es 8.01 | p = 0.00 |
| GA_458 | AF080158 | lkB kinase-b sequence | | 4 | 0 | 0 | 0 | 4 | es > 4 | p = 0.00 |
| GA_4619 | NM_012295 | calcineurin binding protein 1 (CABIN1) | | 6 | 4 | 1 | 0 | 11 | es 3.60 | p = 0.03 |
| GA_4659 | NM_134434 | RAD54B homolog (RAD54B), transcript variant 2 | | 4 | 0 | 2 | 0 | 6 | es 6.01 | p = 0.04 |
| GA_4689 | NM_012470 | transportin-SR (TRN-SR) | | 11 | 4 | 3 | 1 | 19 | es 4.13 | p = 0.00 |
| GA_4693 | NM_012256 | zinc finger protein 212 (ZNF212) | [symbol] | 5 | 0 | 1 | 2 | 8 | es 5.01 | p = 0.03 |
| GA_4694 | NM_012482 | zinc finger protein 281 (ZNF281) | [symbol] | 4 | 0 | 0 | 0 | 4 | es > 4 | p = 0.00 |
| GA_4788 | NM_016263 | Fzr1 protein (FZR1) | | 5 | 1 | 0 | 3 | 9 | es 3.75 | p = 0.05 |
| GA_4802 | AB033092 | KIAA1266 protein sequence | | 9 | 4 | 2 | 0 | 15 | es 4.51 | p = 0.00 |
| GA_4973 | NM_015503 | SH2-B homolog (SH2B) | | 5 | 2 | 1 | 1 | 9 | es 3.75 | p = 0.05 |
| GA_5037 | AB037847 | KIAA1426 protein sequence | | 6 | 2 | 3 | 0 | 11 | es 3.60 | p = 0.03 |
| GA_5052 | NM_015705 | hypothetical protein DJ1042K10.2 (DJ1042K10.2) | | 9 | 2 | 2 | 1 | 14 | es 5.41 | p = 0.00 |
| GA_5301 | NM_145251 | serine/threonine/tyrosine interacting protein (STYX) | | 4 | 0 | 0 | 0 | 4 | es > 4 | p = 0.00 |
| GA_5391 | NM_002968 | sal-like 1 (Drosophila) (SALL1) | | 7 | 1 | 1 | 0 | 9 | es 10.51 | p = 0.00 |
| GA_5470 | NM_002610 | pyruvate dehydrogenase kinase, isoenzyme 1 | | 4 | 0 | 1 | 1 | 6 | es 6.01 | p = 0.04 |
| | | (PDK1), nuclear gene encoding mitochondrial | | | | | | | | |
| | | protein | | | | | | | | |
| GA_5475 | NM_012280 | FtsJ homolog 1 (E. coli) (FTSJ1) | | 6 | 0 | 1 | 0 | 7 | es 18.02 | p = 0.00 |
| GA_5493 | NM_005415 | solute carrier family 20 (phosphate transporter), | | 6 | 1 | 0 | 3 | 10 | es 4.51 | p = 0.02 |
| | | member 1 (SLC20A1) | | | | | | | | |
| GA_5504 | NM_007318 | presenilin 1 (Alzheimer disease 3) (PSEN1), | | 5 | 1 | 1 | 2 | 9 | es 3.75 | p = 0.05 |
| | | transcript variant I-463 | | | | | | | | |
| GA_5513 | NM_014324 | alpha-methylacyl-CoA racemase (AMACR) | | 4 | 0 | 1 | 0 | 5 | es 12.01 | p = 0.02 |
| GA_5534 | NM_014316 | calcium regulated heat stable protein 1, 24 kDa | | 8 | 1 | 3 | 1 | 13 | es 4.81 | p = 0.01 |
| | | (CARHSP1) | | | | | | | | |
| GA_5620 | NM_014516 | CCR4-NOT transcription complex, subunit 3 | [symbol] | 8 | 5 | 1 | 2 | 16 | es 3.00 | p = 0.04 |
| | | (CNOT3) | | | | | | | | |
| GA_5622 | NM_014434 | NADPH-dependent FMN and FAD containing | | 5 | 0 | 1 | 0 | 6 | es 15.02 | p = 0.00 |
| | | oxidoreductase (NR1) | | | | | | | | |
| GA_5665 | NM_014264 | serine/threonine kinase 18 (STK18) | | 5 | 1 | 1 | 2 | 9 | es 3.75 | p = 0.05 |
| GA_5703 | NM_134264 | SOCS box-containing WD protein SWiP-1 (WSB1), | | 44 | 29 | 9 | 12 | 94 | es 2.64 | p = 0.00 |
| | | transcript variant 3 | | | | | | | | |
| GA_5729 | NM_015456 | cofactor of BRCA1 (COBRA1) | | 7 | 2 | 2 | 0 | 11 | es 5.26 | p = 0.01 |
| GA_5735 | NM_015537 | DKFZP586J1624 protein (DKFZP586J1624) | | 4 | 1 | 0 | 1 | 6 | es 6.01 | p = 0.04 |
| GA_5811 | NM_014669 | KIAA0095 gene product (KIAA0095) | | 10 | 3 | 4 | 0 | 17 | es 4.29 | p = 0.00 |
| GA_5829 | NM_014773 | KIAA0141 gene product (KIAA0141) | | 8 | 1 | 2 | 3 | 14 | es 4.00 | p = 0.01 |
| GA_5836 | NM_014865 | chromosome condensation-related SMC-associated | | 12 | 5 | 4 | 2 | 23 | es 3.28 | p = 0.01 |
| | | protein 1 (KIAA0159) | | | | | | | | |
| | | protein 1 (KIAA0159) | | | | | | | | |
| GA_5906 | NM_014675 | KIAA0445 gene product (KIAA0445) | | 5 | 3 | 1 | 0 | 9 | es 3.75 | p = 0.05 |
| GA_5911 | NM_014857 | KIAA0471 gene product (KIAA0471) | | 4 | 0 | 0 | 2 | 6 | es 6.01 | p = 0.04 |
| GA_5954 | NM_014871 | KIAA0710 gene product (KIAA0710) | | 5 | 2 | 0 | 0 | 7 | es 7.51 | p = 0.01 |
| GA_5961 | NM_014828 | chromosome 14 open reading frame 92 (C14orf92) | | 7 | 3 | 0 | 3 | 13 | es 3.50 | p = 0.02 |
| GA_5981 | NM_014921 | lectomedin-2 (KIAA0821) | | 11 | 5 | 0 | 1 | 17 | es 5.51 | p = 0.00 |
| GA_6007 | NM_014962 | BTB (POZ) domain containing 3 (BTBD3) | | 7 | 0 | 3 | 3 | 13 | es 3.50 | p = 0.02 |
| GA_6011 | NM_014963 | KIAA0963 protein (KIAA0963) | | 4 | 1 | 0 | 0 | 5 | es 12.01 | p = 0.02 |
| GA_6106 | NM_015888 | hook1 protein (HOOK1) | | 5 | 0 | 0 | 1 | 6 | es 15.02 | p = 0.00 |
| GA_6133 | NM_016335 | proline dehydrogenase (oxidase) 1 (PRODH), | | 5 | 1 | 2 | 0 | 8 | es 5.01 | p = 0.03 |
| | | nuclear gene encoding mitochondrial protein | | | | | | | | |
| GA_6139 | NM_016448 | RA-regulated nuclear matrix-associated protein | | 6 | 1 | 2 | 0 | 9 | es 6.01 | p = 0.01 |
| | | (RAMP) | | | | | | | | |
| GA_6232 | NM_016223 | protein kinase C and casein kinase substrate in | | 5 | 1 | 1 | 1 | 8 | es 5.01 | p = 0.03 |
| | | neurons 3 (PACSIN3) | | | | | | | | |
| GA_6271 | NM_016518 | pipecolic acid oxidase (PIPOX) | | 4 | 0 | 0 | 0 | 4 | es > 4 | p = 0.00 |
| GA_6317 | NM_015935 | CGI-01 protein (CGI-01) | | 7 | 2 | 1 | 3 | 13 | es 3.50 | p = 0.02 |
| GA_638 | AB024494 | huntingtin interacting protein 3 sequence | | 4 | 0 | 2 | 0 | 6 | es 6.01 | p = 0.04 |
| GA_6438 | NM_002889 | retinoic acid receptor responder (tazarotene | | 4 | 0 | 0 | 1 | 5 | es 12.01 | p = 0.02 |
| | | induced) 2 (RARRES2) | | | | | | | | |
| GA_6445 | NM_017424 | cat eye syndrome chromosome region, candidate 1 | | 10 | 2 | 2 | 4 | 18 | es 3.75 | p = 0.01 |
| | | (CECR1) | | | | | | | | |
| GA_6460 | NM_017415 | kelch-like 3 (Drosophila) (KLHL3) | | 4 | 0 | 0 | 0 | 4 | es > 4 | p = 0.00 |
| GA_6649 | NM_148956 | Williams Beuren syndrome chromosome region 20A | | 4 | 0 | 0 | 0 | 4 | es > 4 | p = 0.00 |
| | | (WBSCR20A), transcript variant 1 | | | | | | | | |
| GA_6665 | NM_018077 | hypothetical protein FLJ10377 (FLJ10377) | | 7 | 0 | 2 | 3 | 12 | es 4.20 | p = 0.01 |
| GA_6669 | NM_018085 | importin 9 (FLJ10402) | | 12 | 0 | 3 | 3 | 18 | es 6.01 | p = 0.00 |
| GA_6673 | NM_018093 | hypothetical protein FLJ10439 (FLJ10439) | | 5 | 2 | 0 | 2 | 9 | es 3.75 | p = 0.05 |
| GA_6731 | NM_018182 | hypothetical protein FLJ10700 (FLJ10700) | | 7 | 0 | 2 | 1 | 10 | es 7.01 | p = 0.00 |
| GA_6742 | NM_018198 | hypothetical protein FLJ10737 (FLJ10737) | | 8 | 4 | 3 | 0 | 15 | es 3.43 | p = 0.02 |
| GA_6760 | NM_018228 | chromosome 14 open reading frame 115 | | 13 | 1 | 0 | 0 | 14 | es 39.05 | p = 0.00 |
| | | (C14orf115) | | | | | | | | |
| GA_6806 | NM_018303 | homolog of yeast Sec5 (SEC5) | | 5 | 1 | 1 | 1 | 8 | es 5.01 | p = 0.03 |
| GA_6905 | NM_017722 | hypothetical protein FLJ20244 (FLJ20244) | | 4 | 1 | 0 | 1 | 6 | es 6.01 | p = 0.04 |
| GA_6957 | NM_017815 | chromosome 14 open reading frame 94 (C14orf94) | | 4 | 0 | 0 | 1 | 5 | es 12.01 | p = 0.02 |
| GA_6975 | NM_017840 | mitochondrial ribosomal protein L16 (MRPL16), | | 6 | 0 | 2 | 2 | 10 | es 4.51 | p = 0.02 |
| | | nuclear gene encoding mitochondrial protein | | | | | | | | |
| GA_7078 | NM_015148 | PAS domain containing serine/threonine kinase | | 5 | 0 | 0 | 0 | 5 | es > 4 | p = 0.00 |
| | | (PASK) | | | | | | | | |
| GA_7155 | NM_007098 | clathrin, heavy polypeptide-like 1 (CLTCL1), | | 4 | 0 | 1 | 0 | 5 | es 12.01 | p = 0.02 |
| | | transcript variant 2 | | | | | | | | |
| GA_7158 | NM_017489 | telomeric repeat binding factor (NIMA-interacting) 1 | | 14 | 3 | 2 | 3 | 22 | es 5.26 | p = 0.00 |
| | | (TERF1), transcript variant 1 | | | | | | | | |
| GA_7170 | NM_019013 | hypothetical protein FLJ10156 (FLJ10156) | | 7 | 1 | 3 | 2 | 13 | es 3.50 | p = 0.02 |
| GA_7178 | NM_019079 | hypothetical protein FLJ10884 (FLJ10884) | | 34 | 2 | 4 | 1 | 41 | es 14.59 | p = 0.00 |
| GA_7334 | NM_020347 | leucine zipper transcription factor-like 1 (LZTFL1) | [symbol] | 6 | 2 | 1 | 0 | 9 | es 6.01 | p = 0.01 |
| GA_7382 | AB040878 | KIAA1445 protein sequence | | 7 | 1 | 0 | 2 | 10 | es 7.01 | p = 0.00 |
| GA_7542 | | | | 21 | 0 | 4 | 0 | 25 | es 15.77 | p = 0.00 |
| GA_7691 | D42046 | The ha3631 gene product is related to S.cerevisiae | | 4 | 1 | 1 | 0 | 6 | es 6.01 | p = 0.04 |
| | | protein encoded in chromosome VIII. sequence | | | | | | | | |
| GA_8100 | NM_054013 | mannosyl (alpha-1,3-)-glycoprotein beta-1,4-N- | | 5 | 1 | 1 | 2 | 9 | es 3.75 | p = 0.05 |
| | | acetylglucosaminyltransferase, isoenzyme B | | | | | | | | |
| | | (MGAT4B), transcript variant 2 | | | | | | | | |
| GA_8103 | NM_144570 | HN1 like (HN1L) | | 14 | 2 | 4 | 4 | 24 | es 4.20 | p = 0.00 |
| GA_8119 | NM_012266 | DnaJ (Hsp40) homolog, subfamily B, member 5 | | 4 | 1 | 0 | 1 | 6 | es 6.01 | p = 0.04 |
| | | (DNAJB5) | | | | | | | | |
| GA_8152 | AK095108 | cDNA FLJ37789 fis, clone BRHIP3000081 | | 6 | 2 | 1 | 0 | 9 | es 6.01 | p = 0.01 |
| | | sequence | | | | | | | | |
| GA_82 | NM_015545 | KIAA0632 protein (KIAA0632) | | 5 | 1 | 1 | 1 | 8 | es 5.01 | p = 0.03 |
| GA_8484 | AK026658 | cDNA: FLJ23005 fis, clone LNG00396, highly similar | | 4 | 0 | 0 | 0 | 4 | es > 4 | p = 0.00 |
| | | to AF055023clone 24723 mRNA sequence | | | | | | | | |
| GA_8559 | NM_022497 | mitochondrial ribosomal protein S25 (MRPS25), | | 6 | 1 | 3 | 1 | 11 | es 3.60 | p = 0.03 |
| | | nuclear gene encoding mitochondrial protein | | | | | | | | |
| GA_8603 | NM_007175 | chromosome 8 open reading frame 2 (C8orf2) | | 7 | 3 | 1 | 1 | 12 | es 4.20 | p = 0.01 |
| GA_8667 | | | | 4 | 0 | 0 | 0 | 4 | es > 4 | p = 0.00 |
| GA_8686 | Z24725 | mitogen inducible gene mig-2 sequence | | 10 | 3 | 0 | 3 | 16 | es 5.01 | p = 0.00 |
| GA_8730 | AK098833 | cDNA FLJ25967 fis, clone CBR01929 sequence | | 10 | 3 | 2 | 0 | 15 | es 6.01 | p = 0.00 |
| GA_8803 | NM_000533 | proteolipid protein 1 (Pelizaeus-Merzbacher | | 6 | 3 | 0 | 0 | 9 | es 6.01 | p = 0.01 |
| | | disease, | | | | | | | | |
| | | spastic paraplegia 2, uncomplicated) (PLP1) | | | | | | | | |
| GA_8862 | AK091593 | cDNA FLJ34274 fis, clone FEBRA2003327 | | 5 | 0 | 0 | 0 | 5 | es > 4 | p = 0.00 |
| | | sequence | | | | | | | | |
| GA_9014 | | | | 6 | 0 | 1 | 1 | 8 | es 9.01 | p = 0.00 |
| GA_9162 | AF311912 | pancreas tumor-related protein sequence | | 7 | 1 | 0 | 4 | 12 | es 4.20 | p = 0.01 |
| GA_9163 | NM_138639 | BCL2-like 12 (proline rich) (BCL2L12), transcript | | 8 | 1 | 3 | 0 | 12 | es 6.01 | p = 0.00 |
| | | variant 1 | | | | | | | | |
| GA_9167 | AF308602 | NOTCH 1 sequence | | 6 | 2 | 1 | 0 | 9 | es 6.01 | p = 0.01 |
| GA_9183 | NM_007129 | Zic family member 2 (odd-paired homolog, | [symbol] | 8 | 1 | 1 | 0 | 10 | es 12.01 | p = 0.00 |
| | | Drosophila) (ZIC2) | | | | | | | | |
| GA_9257 | NM_005088 | DNA segment on chromosome X and Y (unique) | | 4 | 1 | 0 | 1 | 6 | es 6.01 | p = 0.04 |
| | | 155 expressed sequence (DXYS155E) | | | | | | | | |
| GA_9338 | NM_020436 | similar to SALL1 (sal (Drosophila)-like (LOC57167) | | 11 | 2 | 3 | 0 | 16 | es 6.61 | p = 0.00 |
| GA_9365 | NM_021078 | GCN5 general control of amino-acid synthesis 5-like | | 7 | 1 | 2 | 1 | 11 | es 5.26 | p = 0.01 |
| | | 2 (yeast) (GCN5L2) | | | | | | | | |
| GA_9384 | NM_020997 | left-right determination, factor B (LEFTB) | | 4 | 0 | 1 | 0 | 5 | es 12.01 | p = 0.02 |
| GA_9388 | NM_021643 | GS3955 protein (GS3955) | | 7 | 1 | 0 | 2 | 10 | es 7.01 | p = 0.00 |
| GA_9488 | NM_007372 | RNA helicase-related protein (RNAHP) | | 12 | 7 | 1 | 6 | 26 | es 2.57 | p = 0.02 |
| GA_9571 | NM_022130 | golgi phosphoprotein 3 (coat-protein) (GOLPH3) | | 6 | 2 | 2 | 1 | 11 | es 3.60 | p = 0.03 |
| GA_9593 | NM_022372 | G protein beta subunit-like (GBL) | | 6 | 0 | 1 | 1 | 8 | es 9.01 | p = 0.00 |
| GA_96 | NM_012297 | Ras-GTPase activating protein SH3 domain-binding | | 19 | 9 | 6 | 8 | 42 | es 2.48 | p = 0.00 |
| | | protein 2 (KIAA0660) | | | | | | | | |
| GA_9664 | NM_015339 | activity-dependent neuroprotector (ADNP) | | 7 | 1 | 2 | 2 | 12 | es 4.20 | p = 0.01 |
| GA_9688 | NM_022767 | hypothetical protein FLJ12484 (FLJ12484) | | 14 | 3 | 1 | 3 | 21 | es 6.01 | p = 0.00 |
| GA_9697 | NM_022778 | hypothetical protein DKFZp434L0117 | | 6 | 2 | 1 | 0 | 9 | es 6.01 | p = 0.01 |
| | | (DKFZP434L0117) | | | | | | | | |
| GA_9784 | NM_021873 | cell division cycle 25B (CDC25B), transcript variant 3 | | 5 | 2 | 0 | 1 | 8 | es 5.01 | p = 0.03 |
| GA_9829 | BM454622 | AGENCOURT_6406365 NIH_MGC_92cDNA clone | | 6 | 1 | 1 | 0 | 8 | es 9.01 | p = 0.00 |
| | | IMAGE: 5583082 5′ sequence | | | | | | | | |
| GA_9952 | BC003542 | Unknown (protein for IMAGE: 3611719) sequence | | 6 | 0 | 1 | 0 | 7 | es 18.02 | p = 0.00 |
| GA_9996 | NM_005911 | methionine adenosyltransferase II, alpha (MAT2A) | | 27 | 8 | 9 | 14 | 58 | es 2.62 | p = 0.00 |

TABLE 6
EST Frequency of Genes that Up-regulate upon Differentiation

| | | | | EST counts | | | | | | |
|---|---|---|---|---|---|---|---|---|---|---|
| Geron ID | GenBank ID | Name | | ES | EB | preHEP | preNeu | Total | Relative Expression | |
| GA_10484 | AK056774 | unnamed protein product sequence | | 4 | 153 | 17 | 34 | 208 | es 0.06 | p = 0.00 |
| GA_10493 | NM_023009 | MARCKS-like protein (MLP) | | 6 | 7 | 15 | 32 | 60 | es 0.33 | p = 0.01 |
| GA_1071 | NM_001641 | APEX nuclease (multifunctional DNA repair | | 5 | 13 | 15 | 12 | 45 | es 0.38 | p = 0.04 |
| | | enzyme) 1 (APEX1), transcript variant 1 | | | | | | | | |
| GA_11334 | NM_032272 | homolog of yeast MAF1 (MAF1) | | 0 | 4 | 7 | 1 | 12 | es 0.00 | p = 0.05 |
| GA_11407 | NM_015070 | KIAA0853 protein (KIAA0853) | | 0 | 2 | 2 | 8 | 12 | es 0.00 | p = 0.05 |
| GA_12217 | BC009917 | Unknown (protein for MGC: 2764) sequence | | 0 | 7 | 3 | 5 | 15 | es 0.00 | p = 0.03 |
| GA_1222 | NM_001901 | connective tissue growth factor(CTGF) | | 2 | 26 | 4 | 14 | 46 | es 0.14 | p = 0.00 |
| GA_12727 | NM_004926 | zinc finger protein 36, C3H type-like 1 (ZFP36L1) | [symbol] | 3 | 8 | 12 | 22 | 45 | es 0.21 | p = 0.00 |
| GA_1336 | NM_002024 | fragile X mental retardation 1 (FMR1) | [symbol] | 0 | 3 | 4 | 7 | 14 | es 0.00 | p = 0.03 |
| GA_1353 | NM_002051 | GATA binding protein 3 (GATA3) | [symbol] | 0 | 2 | 8 | 2 | 12 | es 0.00 | p = 0.05 |
| GA_1403 | NM_001530 | hypoxia-inducible factor 1, alpha subunit (basic | [symbol] | 4 | 22 | 5 | 8 | 39 | es 0.34 | p = 0.04 |
| | | helix-loop-helix transcription factor) (HIF1A) | | | | | | | | |
| GA_1432 | NM_002166 | inhibitor of DNA binding 2, dominant negative helix- | | 1 | 3 | 17 | 4 | 25 | es 0.13 | p = 0.01 |
| | | loop-helix protein (ID2) | [symbol] | | | | | | | |
| GA_1476 | NM_002276 | keratin 19 (KRT19) | | 1 | 26 | 14 | 38 | 79 | es 0.04 | p = 0.00 |
| GA_1545 | NM_002512 | non-metastatic cells 2, protein (NM23B) expressed | | 3 | 6 | 7 | 16 | 32 | es 0.31 | p = 0.04 |
| | | in (NME2), nuclear gene encoding mitochondrial | | | | | | | | |
| | | protein | | | | | | | | |
| GA_1556 | NM_003633 | ectodermal-neural cortex (with BTB-like domain) | | 1 | 5 | 2 | 28 | 36 | es 0.09 | p = 0.00 |
| | | (ENC1) | | | | | | | | |
| GA_1735 | NM_002806 | proteasome (prosome, macropain) 26S subunit, | | 1 | 7 | 7 | 8 | 23 | es 0.14 | p = 0.03 |
| | | ATPase, 6 (PSMC6) | | | | | | | | |
| GA_1736 | NM_002814 | proteasome (prosome, macropain) 26S subunit, | | 0 | 4 | 10 | 5 | 19 | es 0.00 | p = 0.01 |
| | | non-ATPase, 10 (PSMD10) | | | | | | | | |
| GA_1841 | NM_000979 | ribosomal protein L18 (RPL18) | | 4 | 6 | 36 | 35 | 81 | es 0.16 | p = 0.00 |
| GA_1843 | NM_000982 | ribosomal protein L21 (RPL21) | | 1 | 7 | 48 | 42 | 98 | es 0.03 | p = 0.00 |
| GA_1850 | BC020169 | clone IMAGE: 3543815, partial cds | | 0 | 2 | 8 | 11 | 21 | es 0.00 | p = 0.00 |
| GA_1857 | NM_000999 | ribosomal protein L38 (RPL38) | | 1 | 2 | 12 | 10 | 25 | es 0.13 | p = 0.01 |
| GA_1866 | NM_002950 | ribophorin I (RPN1) | | 3 | 12 | 10 | 14 | 39 | es 0.25 | p = 0.01 |
| GA_1886 | NM_001009 | ribosomal protein S5 (RPS5) | | 8 | 14 | 46 | 30 | 98 | es 0.27 | p = 0.00 |
| GA_1977 | NM_003134 | signal recognition particle 14 kDa (homologous Alu | | 1 | 4 | 18 | 12 | 35 | es 0.09 | p = 0.00 |
| | | RNA binding protein) (SRP14) | | | | | | | | |
| GA_2014 | NM_003564 | transgelin 2 (TAGLN2) | | 5 | 31 | 8 | 28 | 72 | es 0.22 | p = 0.00 |
| GA_2039 | NM_003246 | thrombospondin 1 (THBS1) | | 0 | 3 | 2 | 7 | 12 | es 0.00 | p = 0.05 |
| GA_23018 | NM_005336 | high density lipoprotein binding protein; vigilin | | 11 | 37 | 17 | 21 | 86 | es 0.44 | p = 0.01 |
| | | sequence | | | | | | | | |
| GA_23176 | | | | 2 | 18 | 3 | 7 | 30 | es 0.21 | p = 0.02 |
| GA_23180 | AB009010 | polyubiquitin UbC, complete cds | | 7 | 16 | 23 | 26 | 72 | es 0.32 | p = 0.00 |
| GA_23653 | NM_003289 | tropomyosin 2 (beta) (TPM2) | | 2 | 14 | 7 | 8 | 31 | es 0.21 | p = 0.01 |
| GA_23969 | | | | 0 | 1 | 181 | 20 | 202 | es 0.00 | p = 0.00 |
| GA_24037 | | | | 0 | 1 | 6 | 5 | 12 | es 0.00 | p = 0.05 |
| GA_2524 | NM_004415 | desmoplakin (DPI, DPII) (DSP) | | 3 | 14 | 5 | 23 | 45 | es 0.21 | p = 0.00 |
| GA_2597 | NM_138610 | H2A histone family, member Y (H2AFY), transcript | | 1 | 5 | 5 | 14 | 25 | es 0.13 | p = 0.01 |
| | | variant 3 | | | | | | | | |
| GA_2627 | NM_004905 | anti-oxidant protein 2 (non-selenium glutathione | | 3 | 6 | 11 | 17 | 37 | es 0.27 | p = 0.01 |
| | | peroxidase, acidic calcium-independent | | | | | | | | |
| | | phospholipase A2) (AOP2) | | | | | | | | |
| GA_2702 | NM_000942 | peptidylprolyl isomerase B (cyclophilin B) (PPIB) | | 5 | 6 | 7 | 26 | 44 | es 0.39 | p = 0.04 |
| GA_2752 | NM_004175 | small nuclear ribonucleoprotein D3 polypeptide | | 0 | 1 | 9 | 4 | 14 | es 0.00 | p = 0.03 |
| | | 18 kDa (SNRPD3) | | | | | | | | |
| GA_2782 | NM_004786 | thioredoxin-like, 32 kDa (TXNL) | | 0 | 4 | 1 | 10 | 15 | es 0.00 | p = 0.03 |
| GA_2808 | NM_001154 | annexin A5 (ANXA5) | | 2 | 14 | 4 | 11 | 31 | es 0.21 | p = 0.01 |
| GA_2968 | BC007090 | histidine triad nucleotide-binding protein, clone | | 0 | 1 | 11 | 9 | 21 | es 0.00 | p = 0.00 |
| | | MGC: 14708 IMAGE: 4250172, complete cds | | | | | | | | |
| GA_3016 | NM_001873 | carboxypeptidase E (CPE) | | 1 | 8 | 4 | 9 | 22 | es 0.14 | p = 0.02 |
| GA_3026 | NM_005722 | ARP2 actin-related protein 2 homolog (yeast) | | 6 | 19 | 7 | 19 | 51 | es 0.40 | p = 0.03 |
| | | (ACTR2) | | | | | | | | |
| GA_3033 | NM_005717 | actin related protein 2/3 complex, subunit 5, 16 kDa | | 3 | 10 | 8 | 19 | 40 | es 0.24 | p = 0.01 |
| | | (ARPC5) | | | | | | | | |
| Gk_3036 | NM_152862 | actin related protein 2/3 complex, subunit 2, 34 kDa | | 1 | 9 | 3 | 7 | 20 | es 0.16 | p = 0.04 |
| | | (ARPC2), transcript variant 1 | | | | | | | | |
| GA_3126 | NM_005620 | S100 calcium binding protein A11 (calgizzarin) | | 0 | 1 | 7 | 37 | 45 | es 0.00 | p = 0.00 |
| | | (S100A11) | | | | | | | | |
| GA_3132 | NM_005625 | syndecan binding protein (syntenin) (SDCBP) | | 1 | 3 | 10 | 10 | 24 | es 0.13 | p = 0.02 |
| GA_3260 | NM_006004 | ubiquinol-cytochrome c reductase hinge protein | | 1 | 4 | 12 | 5 | 22 | es 0.14 | p = 0.02 |
| | | (UQCRH) | | | | | | | | |
| GA_3283 | NM_004484 | glypican 3 (GPC3) | | 1 | 6 | 7 | 12 | 26 | es 0.12 | p = 0.01 |
| GA_3294 | NM_006476 | ATP synthase, H+ transporting, mitochondrial F0 | | 0 | 1 | 3 | 11 | 15 | es 0.00 | p = 0.03 |
| | | complex, subunit g (ATP5L) | | | | | | | | |
| GA_33625 | NM_058179 | phosphoserine aminotransferase (PSA), transcript | | 2 | 8 | 5 | 14 | 29 | es 0.22 | p = 0.03 |
| | | variant 1 | | | | | | | | |
| GA_33660 | BF528488 | 602043661F1 NCl_CGAP_Brn67cDNA clone | | 0 | 7 | 7 | 2 | 16 | es 0.00 | p = 0.02 |
| | | IMAGE: 4181462 5′ sequence | | | | | | | | |
| GA_33787 | AL832673 | mRNA; cDNA DKFZp313B1017 (from clone | | 0 | 3 | 4 | 6 | 13 | es 0.00 | p = 0.05 |
| | | DKFZp313B1017) sequence | | | | | | | | |
| GA_3403 | NM_006142 | stratifin (SFN) | | 0 | 2 | 1 | 14 | 17 | es 0.00 | p = 0.01 |
| GA_3431 | NM_006294 | ubiquinol-cytochrome c reductase binding protein | | 0 | 2 | 9 | 7 | 18 | es 0.00 | p = 0.01 |
| | | (UQCRB) | | | | | | | | |
| GA_3435 | NM_006472 | thioredoxin interacting protein (TXNIP) | | 4 | 14 | 16 | 11 | 45 | es 0.29 | p = 0.01 |
| GA_34569 | NM_003299 | tumor rejection antigen (gp96) 1 (TRA1) | | 3 | 9 | 27 | 20 | 59 | es 0.16 | p = 0.00 |
| GA_34776 | NM_002273 | keratin 8 (KRT8) | | 9 | 71 | 144 | 156 | 380 | es 0.07 | p = 0.00 |
| GA_34912 | NM_006367 | adenylyl cyclase-associated protein (CAP) | | 9 | 24 | 10 | 31 | 74 | es 0.42 | p = 0.01 |
| GA_34930 | NM_000700 | annexin A1 (ANXA1) | | 2 | 12 | 3 | 15 | 32 | es 0.20 | p = 0.01 |
| GA_35086 | NM_002128 | high-mobility group box 1 (HMGB1) | | 1 | 3 | 8 | 8 | 20 | es 0.16 | p = 0.04 |
| GA_35179 | NM_001402 | eukaryotic translation elongation factor 1 alpha 1 | | 16 | 29 | 43 | 63 | 151 | es 0.36 | p = 0.00 |
| | | (EEF1A1) | | | | | | | | |
| GA_3530 | NM_002539 | ornithine decarboxylase 1 (ODC1) | | 1 | 10 | 8 | 9 | 28 | es 0.11 | p = 0.01 |
| GA_35369 | NM_003374 | voltage-dependent anion channel 1 (VDAC1) | | 1 | 5 | 6 | 10 | 22 | es 0.14 | p = 0.02 |
| GA_35434 | NM_006094 | deleted in liver cancer 1 (DLC1) | | 0 | 8 | 1 | 5 | 14 | es 0.00 | p = 0.03 |
| GA_35463 | NM_024298 | leukocyte receptor cluster (LRC) member 4 | | 0 | 4 | 9 | 8 | 21 | es 0.00 | p = 0.00 |
| | | (LENG4) | | | | | | | | |
| GA_3560 | NM_003079 | SWI/SNF related, matrix associated, actin | | 2 | 5 | 11 | 11 | 29 | es 0.22 | p = 0.03 |
| | | dependent regulator of chromatin, subfamily e, | | | | | | | | |
| | | member 1 (SMARCE1) | | | | | | | | |
| GA_35641 | BC029424 | similar to weakly similar to glutathione peroxidase 2 | | 1 | 11 | 5 | 3 | 20 | es 0.16 | p = 0.04 |
| | | sequence | | | | | | | | |
| GA_35978 | NM_006830 | ubiquinol-cytochrome c reductase (6.4 kD) subunit | | 0 | 1 | 4 | 7 | 12 | es 0.00 | p = 0.05 |
| | | (UQCR) | | | | | | | | |
| GA_3617 | NM_000391 | ceroid-lipofuscinosis, neuronal 2, late infantile | | 1 | 4 | 15 | 2 | 22 | es 0.14 | p = 0.02 |
| | | (Jansky-Bielschowsky disease) (CLN2) | | | | | | | | |
| GA_36322 | NM_001554 | cysteine-rich, angiogenic inducer, 61 (CYR61) | | 0 | 3 | 3 | 7 | 13 | es 0.00 | p = 0.05 |
| GA_36460 | NM_001300 | core promoter element binding protein (COPEB) | [symbol] | 0 | 6 | 2 | 7 | 15 | es 0.00 | p = 0.03 |
| GA_3652 | NM_005556 | keratin 7 (KRT7) | | 0 | 9 | 1 | 14 | 24 | es 0.00 | p = 0.00 |
| GA_36638 | NM_002954 | ribosomal protein S27a (RPS27A) | | 3 | 5 | 37 | 35 | 80 | es 0.12 | p = 0.00 |
| GA_36721 | NM_005134 | protein phosphatase 4, regulatory subunit 1 | | 0 | 8 | 2 | 6 | 16 | es 0.00 | p = 0.02 |
| | | (PPP4R1) | | | | | | | | |
| GA_36891 | NM_001019 | ribosomal protein S15a (RPS15A) | | 0 | 2 | 50 | 32 | 84 | es 0.00 | p = 0.00 |
| GA_36932 | NM_015338 | KIAA0978 protein (KIAA0978) | | 0 | 5 | 3 | 5 | 13 | es 0.00 | p = 0.05 |
| GA_3707 | NM_003816 | a disintegrin and metalloproteinase domain 9 | | 0 | 8 | 1 | 3 | 12 | es 0.00 | p = 0.05 |
| | | (meltrin gamma) (ADAM9) | | | | | | | | |
| GA_37238 | NM_021019 | myosin, light polypeptide 6, alkali, smooth muscle | | 0 | 2 | 2 | 12 | 16 | es 0.00 | p = 0.02 |
| | | and non-muscle (MYL6), transcript variant 1 | | | | | | | | |
| GA_37377 | NM_000516 | GNAS complex locus (GNAS), transcript variant 1 | [symbol] | 12 | 16 | 27 | 38 | 93 | es 0.44 | p = 0.01 |
| GA_37494 | NM_001305 | claudin 4 (CLDN4) | | 1 | 2 | 10 | 12 | 25 | es 0.13 | p = 0.01 |
| GA_37508 | NM_000994 | ribosomal protein L32 (RPL32) | | 2 | 6 | 26 | 35 | 69 | es 0.09 | p = 0.00 |
| GA_37557 | NM_152437 | hypothetical protein DKFZp761B128 | | 1 | 7 | 13 | 3 | 24 | es 0.13 | p = 0.02 |
| | | (DKFZp761B128) | | | | | | | | |
| GA_37660 | NM_001749 | calpain, small subunit 1 (CAPNS1) | | 4 | 7 | 11 | 20 | 42 | es 0.32 | p = 0.02 |
| GA_37689 | AK022962 | cDNA FLJ12900 fis, clone NT2RP2004321 | | 0 | 4 | 6 | 2 | 12 | es 0.00 | p = 0.05 |
| | | sequence | | | | | | | | |
| GA_37776 | NM_000366 | tropomyosin 1 (alpha) (TPM1) | | 24 | 46 | 37 | 74 | 181 | es 0.46 | p = 0.00 |
| GA_3782 | NM_003968 | ubiquitin-activating enzyme E1C (UBA3 homolog, | | 0 | 1 | 5 | 6 | 12 | es 0.00 | p = 0.05 |
| | | yeast) (UBE1C) | | | | | | | | |
| GA_3789 | NM_006818 | ALL1-fused gene from chromosome 1q (AF1Q) | | 0 | 17 | 1 | 11 | 29 | es 0.00 | p = 0.00 |
| GA_38037 | NM_033480 | F-box only protein 9 (FBXO9), transcript variant 2 | | 0 | 4 | 4 | 4 | 12 | es 0.00 | p = 0.05 |
| GA_3812 | NM_006854 | KDEL (Lys-Asp-Glu-Leu) endoplasmic reticulum | | 3 | 12 | 5 | 17 | 37 | es 0.27 | p = 0.01 |
| | | protein retention receptor 2 (KDELR2) | | | | | | | | |
| GA_38124 | NM_000269 | non-metastatic cells 1, protein (NM23A) expressed | | 1 | 2 | 8 | 13 | 24 | es 0.13 | p = 0.02 |
| | | in (NME1) | | | | | | | | |
| GA_38191 | NM_000224 | keratin 18 (KRT18) | | 8 | 46 | 50 | 119 | 223 | es 0.11 | p = 0.00 |
| GA_38341 | NM_006931 | solute carrier family 2 (facilitated glucose | | 28 | 49 | 45 | 85 | 207 | es 0.47 | p = 0.00 |
| | | transporter), member 3 (SLC2A3) | | | | | | | | |
| GA_38503 | NM_000612 | insulin-like growth factor 2 (somatomedin A) (IGF2) | | 0 | 17 | 4 | 21 | 42 | es 0.00 | p = 0.00 |
| GA_38528 | NM_012062 | dynamin 1-like (DNM1L), transcript variant 1 | | 0 | 5 | 4 | 3 | 12 | es 0.00 | p = 0.05 |
| GA_38545 | NM_005801 | putative translation initiation factor (SUI1) | | 1 | 14 | 15 | 19 | 49 | es 0.06 | p = 0.00 |
| GA_38563 | NM_021005 | nuclear receptor subfamily 2, group F, member 2 | [symbol] | 0 | 9 | 8 | 9 | 26 | es 0.00 | p = 0.00 |
| | | (NR2F2) | | | | | | | | |
| GA_3857 | NM_006644 | heat shock 105 kD (HSP105B) | | 1 | 11 | 3 | 7 | 22 | es 0.14 | p = 0.02 |
| GA_38570 | NM_033150 | collagen, type II, alpha 1 (primary osteoarthritis, | | 0 | 15 | 31 | 5 | 51 | es 0.00 | p = 0.00 |
| | | spondyloepiphyseal dysplasia, congenital) | | | | | | | | |
| | | (COL2A1), transcript variant 2 | | | | | | | | |
| GA_38790 | NM_001743 | calmodulin 2 (phosphorylase kinase, delta) | | 15 | 23 | 36 | 37 | 111 | es 0.47 | p = 0.00 |
| | | (CALM2) | | | | | | | | |
| GA_38817 | NM_013341 | hypothetical protein PTD004 (PTD004) | | 0 | 4 | 5 | 3 | 12 | es 0.00 | p = 0.05 |
| GA_38830 | NM_006013 | ribosomal protein L10 (RPL10) | | 12 | 13 | 71 | 81 | 177 | es 0.22 | p = 0.00 |
| GA_3892 | NM_006888 | calmodulin 1 (phosphorylase kinase, delta) | | 1 | 3 | 11 | 9 | 24 | es 0.13 | p = 0.02 |
| | | (CALM1) | | | | | | | | |
| GA_3973 | NM_144497 | A kinase (PRKA) anchor protein (gravin) 12 | | 0 | 17 | 1 | 20 | 38 | es 0.00 | p = 0.00 |
| | | (AKAP12), transcript variant 2 | | | | | | | | |
| GA_3977 | NM_005139 | annexin A3 (ANXA3) | | 0 | 3 | 4 | 10 | 17 | es 0.00 | p = 0.01 |
| GA_4045 | NM_003897 | immediate early response 3 (IER3), transcript | | 1 | 14 | 2 | 4 | 21 | es 0.15 | p = 0.04 |
| | | variant short | | | | | | | | |
| GA_4132 | NM_002305 | lectin, galactoside-binding, soluble, 1 (galectin 1) | | 0 | 5 | 2 | 7 | 14 | es 0.00 | p = 0.03 |
| | | (LGALS1) | | | | | | | | |
| GA_4182 | NM_001202 | bone morphogenetic protein 4 (BMP4), transcript | | 0 | 7 | 6 | 4 | 17 | es 0.00 | p = 0.01 |
| | | variant 1 | | | | | | | | |
| GA_4395 | NM_003145 | signal sequence receptor, beta (translocon- | | 6 | 17 | 12 | 14 | 49 | es 0.42 | p = 0.05 |
| | | associated protein beta) (SSR2) | | | | | | | | |
| GA_4418 | NM_004800 | transmembrane 9 superfamily member 2 (TM9SF2) | | 0 | 7 | 2 | 8 | 17 | es 0.00 | p = 0.01 |
| GA_4615 | NM_012286 | MORF-related gene X (MRGX) | | 10 | 22 | 16 | 23 | 71 | es 0.49 | p = 0.04 |
| GA_4640 | NM_012342 | putative transmembrane protein (NMA) | | 1 | 8 | 3 | 10 | 22 | es 0.14 | p = 0.02 |
| GA_4914 | NM_016282 | adenylate kinase 3 like 1 (AK3L1) | | 0 | 2 | 6 | 4 | 12 | es 0.00 | p = 0.05 |
| GA_5243 | NM_139207 | nucleosome assembly protein 1-like 1 (NAP1L1), | | 7 | 19 | 28 | 25 | 79 | es 0.29 | p = 0.00 |
| | | transcript variant 1 | | | | | | | | |
| GA_5387 | NM_002047 | glycyl-tRNA synthetase (GARS) | | 8 | 9 | 34 | 34 | 85 | es 0.31 | p = 0.00 |
| GA_5557 | NM_014211 | gamma-aminobutyric acid (GABA) A receptor, pi | | 1 | 3 | 4 | 13 | 21 | es 0.15 | p = 0.04 |
| | | (GABRP) | | | | | | | | |
| GA_5730 | NM_015641 | testis derived transcript (3 LIM domains) (TES), | | 0 | 2 | 2 | 9 | 13 | es 0.00 | p = 0.05 |
| | | transcript variant 1 | | | | | | | | |
| GA_5992 | NM_014899 | Rho-related BTB domain containing 3 (RHOBTB3) | | 0 | 10 | 7 | 13 | 30 | es 0.00 | p = 0.00 |
| GA_6118 | NM_016403 | hypothetical protein HSPC148 (HSPC148) | | 0 | 2 | 7 | 3 | 12 | es 0.00 | p = 0.05 |
| GA_6136 | NM_016368 | myo-inositol 1-phosphate synthase A1 (ISYNA1) | | 1 | 7 | 5 | 16 | 29 | es 0.11 | p = 0.00 |
| GA_6165 | NM_015853 | ORF (LOC51035) | | 1 | 5 | 9 | 5 | 20 | es 0.16 | p = 0.04 |
| GA_6219 | NM_016139 | 16.7 Kd protein (LOC51142) | | 1 | 5 | 13 | 14 | 33 | es 0.09 | p = 0.00 |
| GA_6381 | NM_016641 | membrane interacting protein of RGS16 (MIR16) | | 0 | 2 | 3 | 7 | 12 | es 0.00 | p = 0.05 |
| GA_6388 | NM_016145 | PTD008 protein (PTD008) | | 0 | 1 | 2 | 10 | 13 | es 0.00 | p = 0.05 |
| GA_6437 | NM_016732 | RNA binding protein (autoantigenic, hnRNP- | | 2 | 6 | 7 | 12 | 27 | es 0.24 | p = 0.04 |
| | | associated with lethal yellow) (RALY), transcript | | | | | | | | |
| | | variant 1 | | | | | | | | |
| GA_6481 | NM_014380 | nerve growth factor receptor (TNFRSF16) | | 1 | 4 | 8 | 17 | 30 | es 0.10 | p = 0.00 |
| | | associated protein 1 (NGFRAP1) | | | | | | | | |
| GA_7280 | NM_020199 | HTGN29 protein (HTGN29) | | 0 | 6 | 2 | 6 | 14 | es 0.00 | p = 0.03 |
| GA_7286 | NM_172316 | Meis1, myeloid ecotropic viral integration site 1 | | 0 | 4 | 2 | 10 | 16 | es 0.00 | p = 0.02 |
| | | homolog 2 (mouse) (MEIS2), transcript variant h | | | | | | | | |
| GA_749 | BC015794 | Unknown (protein for MGC: 8837) sequence | | 0 | 4 | 4 | 9 | 17 | es 0.00 | p = 0.01 |
| GA_7520 | NM_003486 | solute carrier family 7 (cationic amino acid | | 2 | 20 | 3 | 20 | 45 | es 0.14 | p = 0.00 |
| | | transporter, y+ system), member 5 (SLC7A5) | | | | | | | | |
| GA_7635 | NM_170746 | selenoprotein H (SELH) | | 0 | 1 | 10 | 2 | 13 | es 0.00 | p = 0.05 |
| GA_8275 | NM_012203 | glyoxylate reductase/hydroxypyruvate reductase | | 0 | 3 | 2 | 12 | 17 | es 0.00 | p = 0.01 |
| | | (GRHPR) | | | | | | | | |
| GA_8627 | NM_006868 | RAB31, member RAS oncogene family (RAB31) | | 0 | 5 | 1 | 7 | 13 | es 0.00 | p = 0.05 |
| GA_8674 | NM_000598 | insulin-like growth factor binding protein 3 (IGFBP3) | | 1 | 15 | 4 | 3 | 23 | es 0.14 | p = 0.03 |
| GA_8980 | NM_005347 | heat shock 70 kDa protein 5 (glucose-regulated | | 10 | 29 | 15 | 30 | 84 | es 0.41 | p = 0.01 |
| | | protein, 78 kDa) (HSPA5) | | | | | | | | |
| GA_9152 | NM_005324 | H3 histone, family 3B (H3.3B) (H3F3B) | | 20 | 26 | 57 | 49 | 152 | es 0.46 | p = 0.00 |
| GA_9196 | NM_000404 | galactosidase, beta 1 (GLB1), transcript variant | | 0 | 6 | 10 | 7 | 23 | es 0.00 | p = 0.00 |
| | | 179423 | | | | | | | | |
| GA_9251 | NM_004373 | cytochrome c oxidase subunit VIa polypeptide 1 | | 0 | 3 | 7 | 8 | 18 | es 0.00 | p = 0.01 |
| | | (COX6A1), nuclear gene encoding mitochondrial | | | | | | | | |
| | | protein | | | | | | | | |
| GA_9266 | NM_021104 | ribosomal protein L41 (RPL41) | | 6 | 9 | 70 | 75 | 160 | es 0.12 | p = 0.00 |
| GA_9649 | NM_014604 | Tax interaction protein 1 (TIP-1) | | 0 | 8 | 5 | 5 | 18 | es 0.00 | p = 0.01 |
| GA_9734 | NM_022908 | hypothetical protein FLJ12442 (FLJ12442) | | 0 | 3 | 2 | 14 | 19 | es 0.00 | p = 0.01 |

### Example 3

### Microarray Analysis for Other Differentially Expressed Genes

In another series of experiments, the level of gene expression was tested at the mRNA level in microarrays.

Genes were selected from the non-redundant set of gene assemblies from the four cDNA libraries described in Example 1, based on their novelty and possible interest as markers. An additional 7,000 sequence-verified clones were obtained from Research Genetics (Huntsville Ala.) and incorporated into an array with a control set of ˜200 known housekeeping genes. Each clone was grown overnight in 96-well format and DNA purified using the Qiagen 96-well DNA kit. The DNA templates were PCR amplified in 100 μL reactions. PCR product was then purified using the Arraylt™ PCR Purification Kit (Telechem, Sunnyvale Calif.) according to manufacturer instructions. Product was dried down, resuspended in 50% DMSO and Arraylt™ Microprinting solution (Telechem, Sunnyvale Calif.) and arrayed onto GAPS™ amino silane coated slides (Corning Inc., Acton Mass.) using a GMS 417 Arrayer (Affymetrix, Santa Clara, Calif.). After printing, slides were humidified and snap heated, baked at 80° for 4 h, then blocked with succinic anhydride.

Total RNA from undifferentiated ES cells, embryoid body cells (EB), retinoic acid treated (preNeu), and DMSO treated (PreHep) cells S, EB, RA-treated, and DMSO-treated cells (10 μg, 15 μg, and 20 μg for sensitivity) was then reverse transcriptase labeled with Cy3 or Cy5 fluorophores, and competitively hybridized to the microarrays overnight at 42° C. in 50% formamide and Sigma hybridization buffer. Undifferentiated ES RNA was directly and indirectly compared with RNA from all other cell types. Experiments were repeated at least 5 times each, and dye reversed. Stratagene Universal Human Reference RNA (Cat. #740000) was used as the indirect comparator. Arrays were washed repeatedly and scanned using a Genepix™ 4000A microarray scanner (Axon Instruments, Fremont Calif.).

Image processing, data extraction and preliminary quality control were performed using Genepix™ Pro 3.0.6 (Axon Instruments). Quality control calculations involved quantifying overall signal intensities, statistical means and medians of pixel intensities and spot morphologies. Extracted data was further analyzed based on statistical algorithms of signal-to-noise, sensitivity range, and reproducibility. Data was then loaded into the GeneSpring™ database and analysis program. Of particular interest were genes that showed reproducible expression differences of 2-fold in either direction, especially when the change occurred upon differentiation to all three differentiated cell types.

The following table lists genes that were identified as being downregulated or upregulated in their expression level upon differentiation into EB, preHEP, or preNEU cells. EST counts are provided from the data generated in the previous example.

TABLE 7
Microarray Analysis - Genes that Decrease Expression upon Differentiation

| | | | Fold Change | | EST Counts | | | |
|---|---|---|---|---|---|---|---|---|
| Geron ID | GenBank ID | Name | RA | DMSO | ES | EB | preHep | preNeu |
| GA_1674 | NM_002701 | POU domain, class 5, transcription factor | −3.61 | −10.68 | 24 | 1 | 2 | 0 |
| | | 1 (POU5F1) | | | | | | |
| GA_9384 | NM_020997 | left-right determination, factor B (LEFTB) | −4.88 | −5.48 | 4 | 0 | 1 | 0 |
| GA_37788 | NM_133631 | roundabout, axon guidance receptor, | −7.93 | −2.9 | 7 | 4 | 1 | 0 |
| | | homolog 1 | | | | | | |
| GA_12173 | NM_021912 | gamma-aminobutyric acid (GABA) A | −3.37 | −2.16 | 4 | 0 | 0 | 0 |
| | | receptor, beta 3 (GABRB3) | | | | | | |
| GA_37606 | NM_019012 | phosphoinositol 3-phosphate-binding | −2.96 | −9.99 | 4 | 2 | 0 | 0 |
| | | protein-2 (PEPP2) | | | | | | |
| GA_1470 | NM_003740 | potassium channel, subfamily K, member | −2.93 | −2.47 | 4 | 0 | 0 | 1 |
| | | 5 (KCNK5) | | | | | | |
| GA_2937 | NM_005207 | v-crk sarcoma virus CT10 oncogene | −2.29 | −3.78 | 6 | 1 | 0 | 0 |
| | | homolog (avian)-like (CRKL) | | | | | | |
| GA_10513 | NM_033209 | Thy-1 co-transcribed (LOC94105) | −2.21 | −3.39 | 7 | 2 | 2 | 1 |
| GA_36957 | NM_024642 | N-acetylgalactosaminyltransferase 12 | −3.24 | −5.05 | 4 | 0 | 1 | 1 |
| | | (GaINAc-T12) (GALNT12) | | | | | | |
| GA_36420 | NM_001064 | transketolase (Wernicke-Korsakoff | −2.25 | −2.28 | 14 | 17 | 11 | 17 |
| | | syndrome) (TKT) | | | | | | |
| GA_1677 | NM_003712 | phosphatidic acid phosphatase type 2C | −2.46 | −2.71 | 1 | 0 | 0 | 0 |
| | | (PPAP2C) | | | | | | |
| GA_36793 | NM_152295 | threonyl-tRNA synthetase (TARS) | −2.18 | −3.5 | 8 | 4 | 1 | 6 |
| GA_7151 | NM_017488 | adducin 2 (beta) (ADD2), transcript | −4.21 | −2.03 | 4 | 2 | 2 | 0 |
| | | variant beta-4 | | | | | | |
| GA_12053 | NM_001986 | ets variant gene 4 (E1A enhancer binding | −2.76 | −2.04 | 0 | 1 | 0 | 4 |
| | | protein, E1AF) (ETV4) | | | | | | |
| GA_1798 | NM_000964 | retinoic acid receptor, alpha (RARA) | −2.76 | −3.3 | 3 | 2 | 0 | 0 |
| GA_5617 | NM_014502 | nuclear matrix protein NMP200 related to | −2.19 | −2.33 | 5 | 3 | 4 | 2 |
| | | splicing factor PRP19 (NMP200) | | | | | | |
| GA_2753 | NM_000582 | secreted phosphoprotein 1 (osteopontin) | −3.78 | −3.32 | 3 | 6 | 2 | 39 |
| | | (SPP1) | | | | | | |
| GA_7151 | NM_017486 | adducin 2 (beta) (ADD2), transcript | −3.34 | −2.13 | 4 | 2 | 2 | 0 |
| | | variant beta-6a | | | | | | |
| GA_36775 | NM_000918 | procollagen-proline, thyroid hormone | −2.01 | −2.65 | 12 | 28 | 10 | 22 |
| | | binding protein p55) (P4HB) | | | | | | |
| GA_1086 | NM_133436 | asparagine synthetase (ASNS), transcript | −2.27 | −2.53 | 6 | 5 | 3 | 13 |
| | | variant 1 | | | | | | |
| GA_2928 | NM_005163 | v-akt murine thymoma viral oncogene | −2.79 | −3.45 | 2 | 10 | 2 | 5 |
| | | homolog 1 (AKT1) | | | | | | |
| GA_33799 | NM_003250 | thyroid hormone receptor (THRA) | −4.28 | −4.44 | 0 | 2 | 0 | 1 |
| GA_37861 | NM_021784 | forkhead box A2 (FOXA2), transcript | −3.56 | −2.99 | 2 | 0 | 0 | 0 |
| | | variant 1 | | | | | | |
| GA_34109 | NM_002026 | fibronectin 1 (FN1), transcript variant 1 | −2.91 | −2.01 | 17 | 166 | 5 | 27 |
| GA_38641 | NM_004309 | Rho GDP dissociation inhibitor (GDI) | −2.72 | −2.35 | 7 | 8 | 9 | 14 |
| | | alpha (ARHGDIA) | | | | | | |
| GA_33829 | NM_002081 | glypican 1 (GPC1) | −2.61 | −2.32 | 3 | 9 | 4 | 1 |
| GA_5549 | NM_014600 | EH-domain containing 3 (EHD3) | −2.39 | −2.81 | 1 | 5 | 1 | 1 |
| GA_9269 | NM_021074 | NADH dehydrogenase (ubiquinone) | −2.26 | −2.01 | 0 | 0 | 9 | 6 |
| | | flavoprotein 2, 24 kDa (NDUFV2) | | | | | | |
| GA_2934 | NM_005180 | B lymphoma Mo-MLV insertion region | −2.11 | −3.24 | 1 | 2 | 0 | 1 |
| | | (mouse) (BMI1) | | | | | | |
| GA_3522 | NM_002415 | macrophage migration inhibitory factor | −2.04 | −2.05 | 4 | 2 | 8 | 9 |
| | | (glycosylation-inhibiting factor) (MIF) | | | | | | |
| GA_2465 | NM_004364 | CCAAT/enhancer binding protein | −2.79 | −4 | 0 | 1 | 0 | 0 |
| | | (C/EBP), alpha (CEBPA) | | | | | | |
| GA_36793 | NM_152295 | threonyl-tRNA synthetase (TARS) | −5.34 | −2.98 | 8 | 4 | 1 | 6 |
| GA_9259 | NM_005539 | inositol polyphosphate-5-phosphatase, | −4.37 | −6.54 | 1 | 0 | 0 | 2 |
| | | 40 kDa (INPP5A) | | | | | | |
| GA_2232 | NM_001348 | death-associated protein kinase 3 | −2.9 | −3.56 | 3 | 3 | 1 | 2 |
| | | (DAPK3) | | | | | | |
| GA_37240 | NM_007029 | stathmin-like 2 (STMN2) | −4.37 | −2.37 | 0 | 4 | 0 | 1 |
| GA_4617 | NM_012289 | Kelch-like ECH-associated protein 1 | −11.88 | −2.59 | 2 | 4 | 2 | 2 |
| | | (KEAP1) | | | | | | |
| GA_38021 | NM_002111 | huntingtin (Huntington disease) (HD) | −10.84 | −2.16 | 1 | 5 | 0 | 2 |
| GA_9227 | NM_001552 | insulin-like growth factor binding protein 4 | −6.13 | −3.06 | 5 | 4 | 0 | 2 |
| | | (IGFBP4) | | | | | | |
| GA_267 | NM_007041 | arginyltransferase 1 (ATE1) | −3.03 | −3.22 | 1 | 1 | 0 | 2 |
| GA_38392 | NM_006597 | heat shock 70 kDa protein 8 (HSPA8), | −8.8 | −2.7 | 39 | 20 | 48 | 62 |
| | | transcript variant 1 | | | | | | |
| GA_1829 | NM_002936 | ribonuclease H1 (RNASEH1) | −2.81 | −2.11 | 1 | 0 | 1 | 2 |
| GA_9228 | NM_001664 | ras homolog gene family, member A | −3.21 | −2.48 | 11 | 18 | 8 | 17 |
| | | (ARHA) | | | | | | |
| GA_1495 | NM_002347 | lymphocyte antigen 6 complex, locus H | −2.33 | −2.57 | 0 | 0 | 0 | 1 |
| | | (LY6H) | | | | | | |
| GA_3840 | NM_006749 | solute carrier family 20 (phosphate | −5.4 | −2.83 | 0 | 1 | 1 | 3 |
| | | transporter), member 2 (SLC20A2) | | | | | | |
| GA_1045 | NM_001105 | activin A receptor, type I (ACVR1) | −2.7 | −2.37 | 0 | 3 | 1 | 3 |
| GA_36361 | NM_020636 | zinc finger protein 275 (ZNF275) | −4.09 | −2.07 | 0 | 0 | 0 | 3 |
| GA_2445 | NM_004337 | chromosome 8 open reading frame 1 | −3.02 | −2.2 | 1 | 0 | 0 | 0 |
| | | (C8orf1) | | | | | | |
| GA_4652 | NM_012228 | pilin-like transcription factor (PILB) | −2.73 | −2.46 | 0 | 0 | 1 | 0 |
| GA_10567 | NM_025195 | phosphoprotein regulated by mitogenic | −4.74 | −3.64 | 0 | 2 | 0 | 1 |
| | | pathways (C8FW) | | | | | | |
| GA_9258 | NM_005393 | plexin B3 (PLXNB3) | −3.56 | −3.04 | 0 | 2 | 0 | 0 |
| GA_35992 | NM_001402 | eukaryotic translation elongation factor 1 | −5.55 | −2.22 | 419 | 467 | 454 | 428 |
| | | alpha 1 (EEF1A1) | | | | | | |
| GA_33537 | NM_133259 | leucine-rich PPR-motif containing | −2.47 | −3.41 | 8 | 7 | 5 | 3 |
| | | (LRPPRC) | | | | | | |
| GA_6367 | NM_016354 | solute carrier family 21 (organic anion | −2.08 | −3.26 | 0 | 0 | 0 | 1 |
| | | transporter), member 12 (SLC21A12) | | | | | | |
| GA_667 | AB028976 | mRNA for KIAA1053 protein, partial cds | −7.55 | −3.52 | 0 | 2 | 0 | 2 |
| | BQ023180 | NCI_CGAP_PI6 cDNA clone UI-1-BB1p- | −2.96 | −2.1 | | | | |
| | | aui-g-05-0-UI 3' sequence | | | | | | |
| | AA419281 | Soares ovary tumor NbHOT cDNA clone | −3.36 | −2.59 | | | | |
| | | IMAGE: 755641 3' sequence | | | | | | |
| | NM_006604 | ret finger protein-like 3 (RFPL3) | −2.69 | −2.5 | | | | |
| | NM_012155 | echinoderm microtubule associated | −9.82 | −6.65 | | | | |
| | | protein like 2 (EML2) | | | | | | |
| | NM_000160 | glucagon receptor (GCGR) | −3.94 | −2.18 | | | | |
| | NM_003181 | T, brachyury homolog (mouse) (T) | −9.15 | −2.11 | | | | |
| | NM_014620 | homeo box C4 (HOXC4), transcript | −9.54 | −2.1 | | | | |
| | | variant 1 | | | | | | |
| | NM_005583 | lymphoblastic leukemia derived sequence | −4.36 | −2.79 | | | | |
| | | 1 (LYL1) | | | | | | |
| | NM_014310 | RASD family, member 2 (RASD2) | −2.72 | −3.13 | | | | |
| | NM_012467 | tryptase gamma 1 (TPSG1) | −2.63 | −2.55 | | | | |
| | NM_000539 | rhodopsin (opsin 2, rod pigment) (retinitis | −4.84 | −5.53 | | | | |
| | | pigmentosa 4, autosomal dominant) | | | | | | |
| | | (RHO) | | | | | | |
| | NM_021076 | neurofilament, heavy polypeptide (200 kD) | −2.03 | −2.41 | | | | |
| | | (NEFH) | | | | | | |
| | NM_012407 | protein kinase C, alpha binding protein | −5.44 | −2.56 | | | | |
| | | (PRKCABP) | | | | | | |
| | NM_000201 | intercellular adhesion molecule 1 (CD54), | −2.18 | −2.06 | | | | |
| | | human rhinovirus receptor (ICAM1) | | | | | | |

TABLE 8
Microarray Analysis - Genes that Increase Expression upon Differentiation

| | | | Fold Change | | EST Counts | | | |
|---|---|---|---|---|---|---|---|---|
| Geron ID | GenBank ID | Name | RA | DMSO | ES | EB | preHep | preNeu |
| GA_1055 | NM_001134 | alpha-fetoprotein (AFP) | 8.02 | 5.07 | 0 | 4 | 0 | 0 |
| GA_1055 | NM_001134 | alpha-fetoprotein (AFP) | 6.45 | 3.71 | 0 | 4 | 0 | 0 |
| GA_1055 | NM_001134 | alpha-fetoprotein (AFP) | 2.58 | 2.67 | 0 | 4 | 0 | 0 |
| GA_1213 | NM_001884 | cartilage linking protein 1 (CRTL1) | 4.57 | 8.71 | 3 | 1 | 17 | 3 |
| GA_1476 | NM_002276 | keratin 19 (KRT19) | 2.09 | 5.21 | 1 | 26 | 14 | 38 |
| GA_8674 | NM_000598 | insulin-like growth factorn binding protein | 3.16 | 3.59 | 1 | 15 | 4 | 3 |
| | | 3 (IGFBP3) | | | | | | |
| GA_3283 | NM_004484 | glypican 3 (GPC3) | 2.6 | 3.29 | 1 | 6 | 7 | 12 |
| GA_37735 | NM_058178 | neuronal pentraxin receptor (NPTXR) | 3.77 | 4.04 | 1 | 0 | 0 | 1 |
| GA_1280 | NM_001957 | endothelin receptor type A(EDNRA) | 3.05 | 6.37 | 2 | 2 | 1 | 0 |
| GA_37308 | NM_003068 | snail homolog 2 (Drosophila) (SNAI2) | 2.24 | 4.68 | 4 | 3 | 0 | 0 |
| GA_5909 | NM_014851 | KIAA0469 gene product | 2.77 | 2.03 | 3 | 3 | 0 | 1 |
| GA_23450 | XM_027313 | ATP synthase mitochondrial F1 complex | 2.48 | 3.55 | 3 | 1 | 1 | 1 |
| | | assembly factor 1 (ATPAF1), | | | | | | |
| GA_7286 | NM_020119 | likely ortholog of rat zinc-finger antiviral | 2.5 | 3.55 | 1 | 0 | 0 | 0 |
| | | protein (ZAP) | | | | | | |

### Example 4

### Specificity of Expression Confirmed by Real-time PCR

To verify the expression patterns of particular genes of interest at the mRNA level, extracts of undifferentiated hES cells and their differentiated progeny were assayed by real-time PCR. Cells were cultured for 1 week with 0.5% dimethyl sulfoxide (DMSO) or 500 nM retinoic acid (RA). The samples were amplified using sequence-specific primers, and the rate of amplification was correlated with the expression level of each gene in the cell population.

Taqman™ RT-PCR was performed under the following conditions: 1×RT Master Mix (ABI), 300 nM for each primer, and 80 nM of probe, and 10 pg to 100 ng of total RNA in nuclease-free water. The reaction was conducted under default RT-PCR conditions of 48° C. hold for 30 min, 95° C. hold for 10 min, and 40 cycles of 95° C. at 15 sec and 60° C. hold for 1 min. RNA was isolated by a guanidinium isothiocyanate method (RNAeasy™ kit, Qiagen) according to manufacturer's instructions, and subsequently DNAse treated (DNAfree™ kit, Ambion). Gene-specific primers and probes were designed by PrimerExpress™ software (Ver. 1.5, ABI). Probe oligonucleotides were synthesized with the fluorescent indicators 6-carboxytluorescein (FAM) and 6-carboxy-tetramethylrhodamine (TAMRA) at the 5′ and 3′ ends, respectively. Relative quantitation of gene expression between multiple samples was achieved by normalization against endogenous 18S ribosomal RNA (primer and probe from ABI) using the ΔΔC_{T} method of quantitation (ABI). Fold change in expression level was calculated as 2 ^{-ΔΔCT}.

The table below shows the results of this analysis. Since the cells have been cultured in RA and DMSO for a short period, they are at the early stages of differentiation, and the difference in expression level is less dramatic than it would be after further differentiation. Of particular interest for following or modulating the differentiation process are markers that show modified expression within the first week of differentiation by more than 2-fold (*), 5-fold (**), 10-fold (***), or 100-fold (****)

TABLE 9
Quantitative RT-PCR analysis of gene expression in hESC differentiation

| | | | | Fold Change | |
|---|---|---|---|---|---|
| | Geron ID | GenBank ID | Name | RA | DMSO |
| A. | GA_10902 | NM_024504 | Pr domain containing 14 (PRDM14)** | −1.9 | −8.3 |
| | GA_11893 | NM_032805 | Hypothetical protein FLJ14549*** | −2.3 | −10.0 |
| | GA_12318 | NM_032447 | Fibrillin3 | | |
| | GA_1322 | NM_000142 | Fibroblast growth factor receptor 3 precursor | 1.5 | 2.3 |
| | | | (FGFR-3)* | | |
| | GA_1329 | NM_002015 | Forkhead box o1a (foxo1a)* | −1.6 | −2.9 |
| | GA_1470 | NM_003740 | Potassium channel subfamily k member 5 (TASK-2) | −1.6 | 1.0 |
| | GA_1674 | NM_002701 | Octamer-binding transcription factor 3a (OCT-3A) | −3.7 | −7.7 |
| | | | (OCT-4)** | | |
| | GA_2024 | NM_003212 | Teratocarcinoma-derived growth factor 1 | −4.0 | −12.5 |
| | | | (CRIPTO)*** | | |
| | GA_2149 | NM_003413 | Zic family member 3 (ZIC3)** | −1.7 | −5.3 |
| | GA_2334 | NM_000216 | Kallmann syndrome 1 sequence (KAL1)* | −1.1 | −2.5 |
| | GA_23552 | BC027972 | Glypican-2 (cerebroglycan) | −1.5 | −1.2 |
| | GA_2356 | NM_002851 | Protein tyrosine phosphatase, receptor-type, z | −1.7 | −3.3 |
| | | | polypeptide 1 (PTPRZ1)* | | |
| | GA_2367 | NM_003923 | Forkhead box h1 (FOXH1)** | −1.8 | −5.6 |
| | GA_2436 | NM_004329 | Bone morphogenetic protein receptor, type Ia | −2.4 | −2.4 |
| | | | (BMPR1A) (ALK-3)* | | |
| | GA_2442 | NM_004335 | Bone marrow stromal antigen 2 (BST-2) | 1.1 | −1.9 |
| | GA_2945 | NM_005232 | Ephrin type-a receptor 1 (EPHA1) | −1.3 | −1.9 |
| | GA_2962 | NM_005314 | Gastrin-releasing peptide receptor (GRP-R)** | −6.3 | −9.1 |
| | GA_2988 | NM_005397 | Podocalyxin-like (PODXL)* | −2.6 | −4.3 |
| | GA_3337 | NM_006159 | Nell2 (NEL-like protein 2) | −1.3 | −1.3 |
| | GA_3559 | NM_005629 | Solute carrier family 6, member 8 (SLC6A8) | −1.1 | −1.1 |
| | GA_420 | X98834 | Zinc finger protein, HSAL2* | −1.4 | −2.8 |
| | GA_5391 | NM_002968 | Sal-like 1 (SALL1), | 1.4 | −1.3 |
| | GA_6402 | NM_016089 | Krab-zinc finger protein SZF1-1* | −1.8 | −3.1 |
| | GA_9167 | AF308602 | Notch 1 (N1) | 1.3 | 1.0 |
| | GA_9183 | AF193855 | Zinc finger protein of cerebellum ZIC2* | 1.0 | −2.9 |
| | GA_9443 | NM_004426 | Early development regulator 1 (polyhomeotic 1 | −1.8 | −5.6 |
| | | | homolog) (EDR1)** | | |
| B. | GA_9384 | NM_020997 | Left-right determination, factor b (LEFTB)** | −16.7 | −25.0 |
| | GA_12173 | BC010641 | Gamma-aminobutyric acid (GABA) A receptor, | −2.8 | −5.6 |
| | | | beta 3** | | |
| | GA_10513 | NM_033209 | Thy-1 co-transcribed*** | −12.5 | −11.1 |
| | GA_1831 | NM_002941 | Roundabout, axon guidance receptor, homolog 1 | 1.1 | 1.0 |
| | | | (ROBO1), | | |
| | GA_2753 | NM_000582 | Secreted phosphoprotein 1 (osteopontin)*** | −3.8 | −10.0 |
| | GA_32919 | NM_133259 | 130 kDa leucine-rich protein (LRP 130) | −1.9 | −1.9 |
| | GA_28290 | AK055829 | FLJ31267 (acetylglucosaminyltransferase-like | −2.3 | −4.5 |
| | | | protein)* | | |
| C. | GA_28053 | T24677 | EST**** | <−100* | <−100* |
| | GA_26303 | NM_138815 | Hypothetical protein BC018070*** | −3.2 | −10.0 |
| | GA_2028 | NM_003219 | Telomerase reverse transcriptase (TERT)* | −2.1 | −2.3 |

### Example 5

### Selection of Markers for Monitoring ES Cell Differentiation

Genes that undergo up- or down-regulation in expression levels during differentiation are of interest for a variety of different commercial applications, as described earlier. This experiment provides an example in which certain genes were selected as a means to monitor the ability of culture conditions to maintain the undifferentiated cell phenotype—and hence, the pluripotent differentiation capability of the cells.

Particular genes were chosen from those identified as having differential expression patterns, because they are khown or suspected of producing a protein gene product that is expressed at the cell surface, or is secreted. These attributes are helpful, because they allow the condition of the cells to be monitored easily either by antibody staining of the cell surface, or by immunoassay of the culture supernatant. Genes were chosen from the EST database (Groups 1), microarray analysis (Group 2), and other sources (Group 3).

TABLE 10
Additional Genes analyzed by real-time PCR

| | | GenBank or |
|---|---|---|
| | Name | ID No. |
| Group 1 | Bone marrow stromal antigen | NM_004335 |
| | Podocalyxin-like | NM_005397 |
| | Rat GPC/ glypican-2 (cerebroglycan) | TA_5416486 |
| | Potassium channel subfamily k member 5 (TASK-2) | NM_003740 |
| | Notch 1 protein | AF308602 |
| | Teratocarcinoma-derived growth factor 1 (Cripto) | NM_003212 |
| | Nel 1 like / NELL2 (Nel-like protein 2) | NM_006159 |
| | Gastrin releasing peptide receptor | NM_005314 |
| | Bone morphogenetic protein receptor | NM_004329 |
| | ABCG2-ABC transporter | AY017168 |
| | Solute carrier family 6, member 8 (SLC6A8) | NM_005629 |
| | hTERT | NM_003219 |
| | Oct 3/4 octamer-binding transcription factor 3a (oct-3a) (oct-4) | NM_002701 |
| Group 2 | Left-right determination factor b (LEFTB) | NM_020997 |
| | Secreted phosphoprotein 1 (osteopontin) | NM_000582 |
| | Gamma-aminobutyric acid (GABA) A receptor, beta 3 | NM_021912 |
| | Roundabout, axon guidance receptor, homologue 1 (ROBO1), | NM_002941 |
| | Glucagon receptor | NM_00160 |
| | Leucine-rich PPR-motif hum 130 kDa hum130leu 130 kd Leu | M92439 |
| | Thy-1 co-transcribed | NM_033209 |
| | Solute carrier family 21 | NM_016354 |
| | LY6H lymphocyte antigen 6 complex locus H | NM_002347 |
| | Plexin (PLXNB3) | NM_005393 |
| | ICAM | NM_000201 |
| Group 3 | Rhodopsin | NM_000539 |
| | Kallmann syndrome 1 sequence (KAL1) | NM_000216 |
| | Armadillo repeat protein deleted in velo-cardio-facial syndrome | NM_001670 |
| | (ARVCF) | |
| | Ephrin type-a receptor 1 (EPHA1) | NM_005232 |

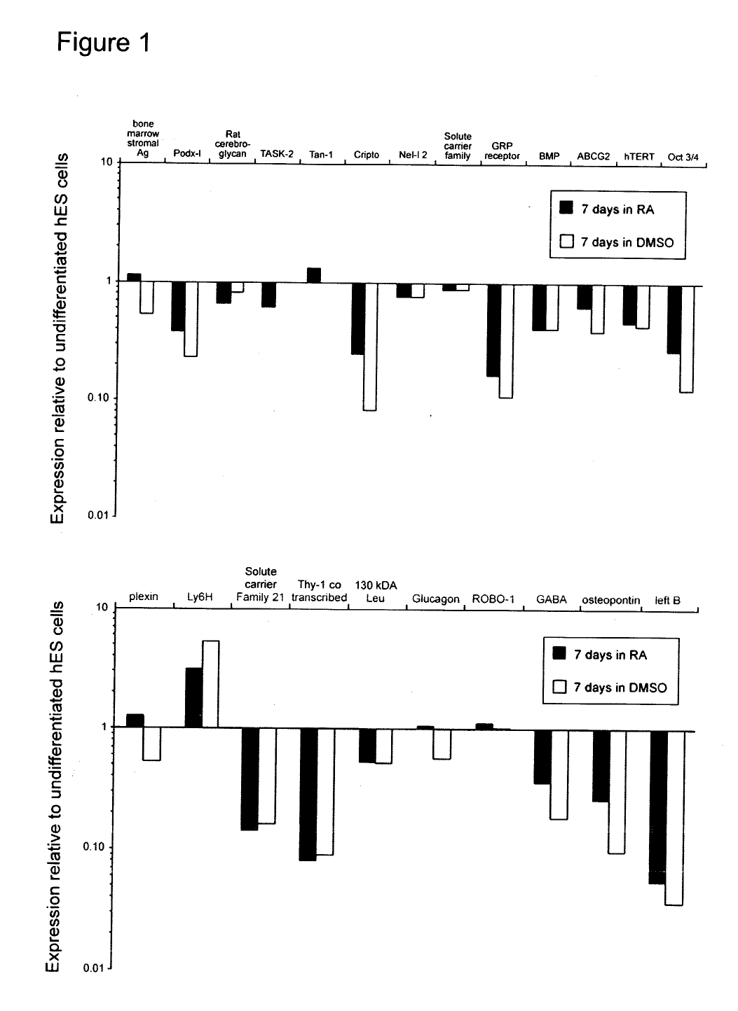

FIG. 1 shows the decrease in expression of the genes in Group I (Upper Panel) and Group II (Lower Panel) in H9 hES cells after culturing for 7 days with RA or DM. Gene expression of rhodopsin and ICAM was below the limit of detection in differentiated cells. KAL1 and EPHA1 were not tested.

Besides hTERT and Oct 3/4, three other genes were selected as characteristic of the undifferentiated hES cell phenotype. They were Teratocarcinoma-derived growth factor (Cripto), Podocalyxin-like (PODXL), and gastrin-releasing peptide receptor (GRPR).

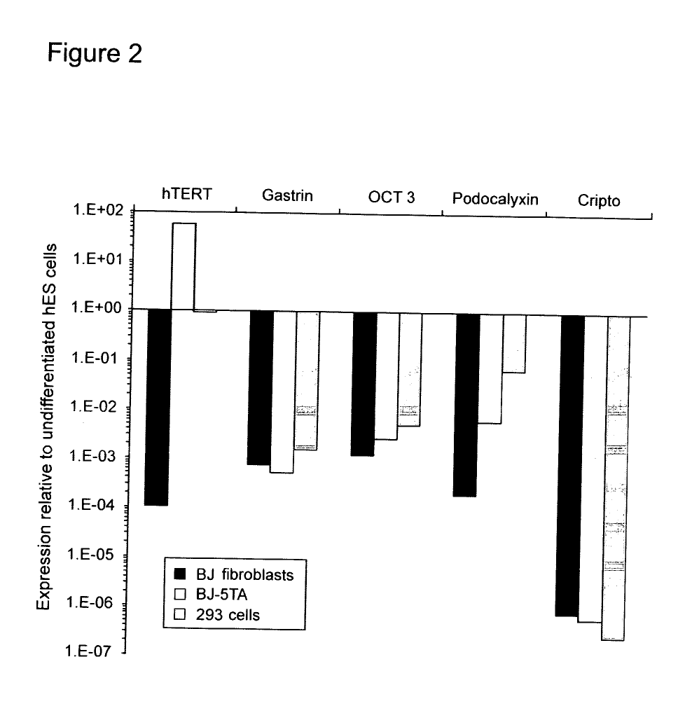

FIG. 2 compares the level of expression of these five genes in hES cells with fully differentiated cells: BJ fibroblasts, BJ fibroblasts transfected to express hTERT (BJ-5TA), and 293 (human embryonic kidney) cells. The level of all markers shown was at least 10-fold higher, and potentially more than 10², 10³, 10⁴, 10⁵, or 10⁶-fold higher in pluripotent stem cells than fully differentiated cells. All five markers retained a detectable level of expression in differentiated cultures of hESC. It is not clear if there is lower level of expression of these markers in differentiated cells, or if the detectable expression derived from the undifferentiated cells in the population. The one exception observed in this experiment was the hTERT transgene, expressed at an elevated level as expected in the BJ-5TA cells.

High-level expression of Cripto, GRPR and PODXL in undifferentiated hES cells reveals interesting aspects of the biology of these cells. Cripto has been implicated in normal mammalian development and tumor growth. Cripto encodes a glycosylphosphoinositol anchored protein that contains an EGF repeat and a cysteine rich motif, which makes it a member of the EGF-CFC family. It has been demonstrated that Cripto serves as a co receptor for Nodal, which is essential for mesoderm and endoderm formation in vertebrate development (Yeo et al., Molecular Cell 7:949, 2001). The finding that Cripto is expressed preferentially on undifferentiated hESC suggests that Nodal is an important signaling molecule for stem cells, perhaps to promote survival and/or proliferation.

PODXL encodes for transmembrane sialoprotein that is physically linked to the cytoskeleton. PODXL is suspected to act as an inhibitor of cell-cell adhesion and has been implicated in the embryonic development of the kidney podocyte. The anti-adhesion properties of PODXL when expressed on undifferentiated hESC may be an important feature related to stem cell migration.

The receptor for gastrin releasing peptide (GRP) is a G-protein coupled receptor that mediates numerous biological effects of Bombesin-like peptides, including regulation of gut acid secretion and satiety. A critical role has also been established for GRP and GRPR in control growth of cultured cells and normal mammalian development. GRP and GRPR may be oncofetal antigens that act as morphogens in normal development and cancer.

### Example 6

### Use of Cell Markers to Modify ES Cell Culture Conditions

This example illustrates the utility of the differentially expressed genes identified according to this invention in the evaluation of culture environments suitable for maintaining pluripotent stem cells.

FIG. 3 show results of an experiment in which hES cells of the H1 line were maintained for multiple passages in different media. Medium conditioned with feeder cells provides factors effective to allow hES cells to proliferate in culture without differentiating. However, culturing in unconditioned medium leads to loss of the undifferentiated phenotype, with an increasing percentage of the cells showing decreased expression of CD9 (a marker for endothelial cells, fibroblasts, and certain progenitor cells), and the classic hES cell marker SSEA-4.

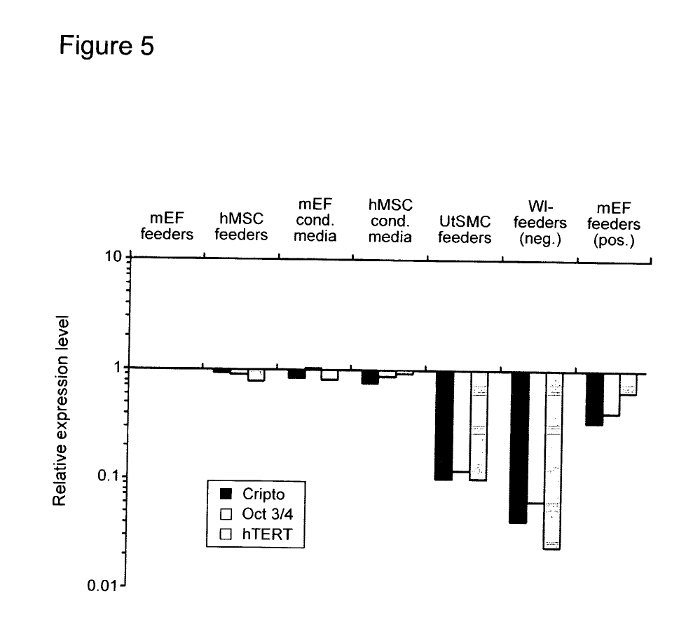

FIG. 4 illustrates the sensitivity of hTERT, Oct 3/4, Cripto, GRP receptor, and podocalyxin-like protein (measured by real-time PCR assay) as a means of determining the degree of differentiation of the cells. After 4 passages in unconditioned X-VIVO™ 10 medium containing 8 ng/mL bFGF, all 5 markers show expression that has been downregulated by about 10-fold. After 8 passages, expression has decreased by 10², 10³, or 10⁴-fold.

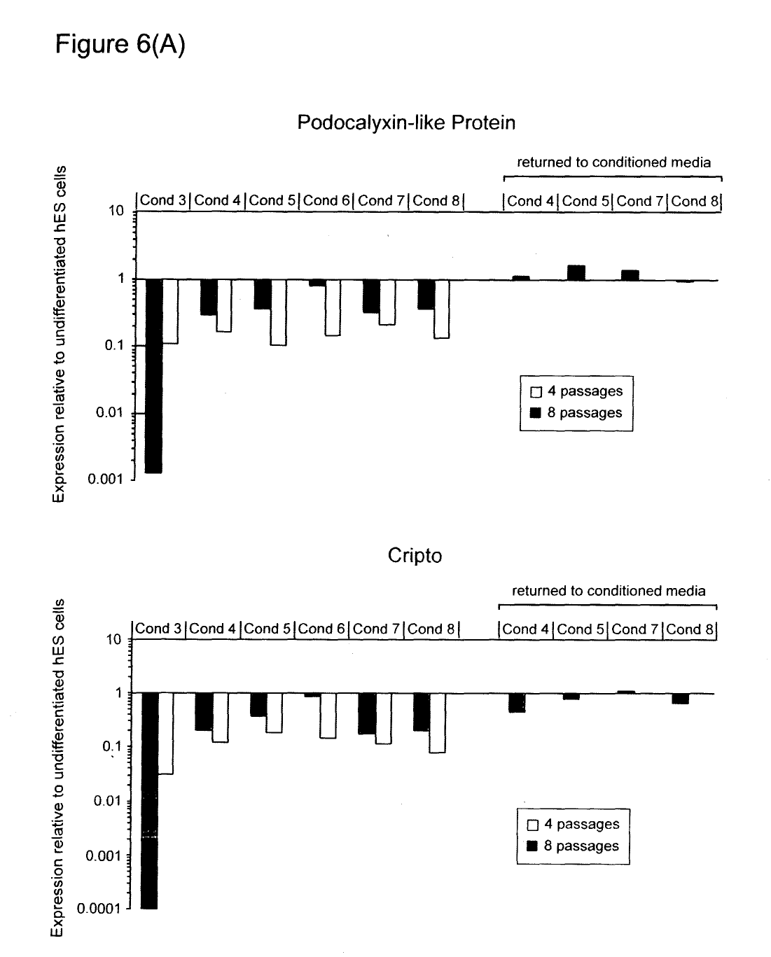

FIG. 5 shows results of an experiment in which the hES cell line H1 was grown on different feeder cell lines: mEF=mouse embryonic fibroblasts; hMSC=human mesenchymal stem cells; UtSMC =human uterine smooth muscle cells; WI-38=an established line of human lung fibroblasts. As monitored by RT-PCR assay of Cripto, Oct 3/4, and hTERT, at least under the conditions used in this experiment, the hMSC are better substitutes for mEF feeders than the other cell lines tested.

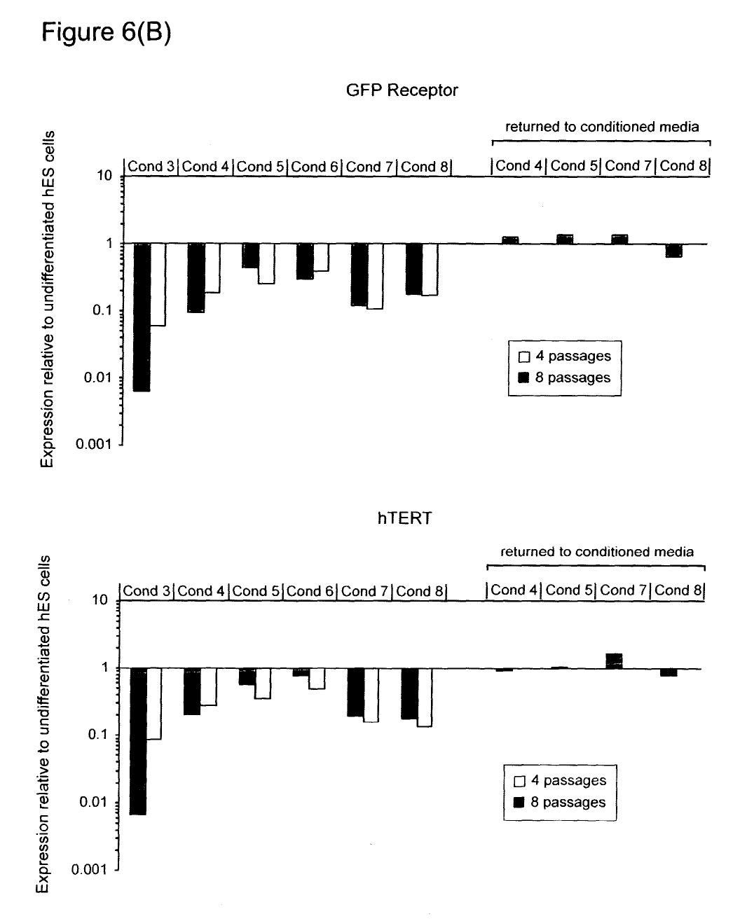

FIG. 6 shows results of an experiment in which different media were tested for their ability to promote growth of hES cells without differentiation. Expression of Podocalyxin-like protein, Cripto, GFP Receptor, and hTERT were measured by RT-PCR. The test media were not preconditioned, but supplemented with the growth factors as follows:

TABLE 11
Growth Conditions Tested for Marker Expression

| | DMEM preconditioned with |
|---|---|
| Standard conditions: | mEF + bFGF (8 ng/mL) |
| Condition 3 | X-VIVO ™ 10 + bFGF (8 ng/mL) |
| Condition 4 | X-VIVO ™ 10 + bFGF (40 ng/mL) |
| Condition 5 | X-VIVO ™ 10 + bFGF (40 ng/mL) + stem cell |
| | factor (SCF, 15 ng/mL) |
| Condition 6 | X-VIVO ™ 10 + bFGF (40 ng/mL) + FIt3 ligand |
| | (75 ng/mL) |
| Condition 7 | X-VIVO ™ 10 + bFGF (40 ng/mL) + LIF |
| | (100 ng/mL) |
| Condition 8 | QBSF ™−60 + bFGF (40 ng/mL) |

The results show that the markers selected to monitor the undifferentiated phenotype showed similar changes in each of these culture conditions. By all criteria, XVIVO 10™ supplemented according to Condition 6 was found to be suitable for culturing hES cells without having to be preconditioned. As shown on the right side, when cells were put back into standard conditioned medium after 8 passages in the test conditions, expression of all four markers returned essentially to original levels. This shows that alterations in expression profiles in media Conditions 4 to 8 are temporary and reversible—consistent with the cells retaining full pluripotency.

### Sequence Data

TABLE 12
Sequences Listed in this Disclosure

| SEQ. ID NO: | Designation | Reference |
|---|---|---|
| 1 | hTERT mRNA sequence | GenBank Accession NM_003129 |
| 2 | hTERT protein sequence | GenBank Accession NM_003129 |
| 3 | Oct 3/4 mRNA sequence | GenBank Accession NM_002701 |
| 4 | Oct 3/4 protein sequence | GenBank Accession NM_002701 |
| 5 | Cripto mRNA sequence | GenBank Accession NM_003212 |
| 6 | Cripto protein sequence | GenBank Accession NM_003212 |
| 7 | podocalyxin-like protein mRNA sequence | GenBank Accession NM_005397 |
| 8 | podocalyxin-like protein amino acid sequence | GenBank Accession NM_005397 |
| 9 | GRP receptor mRNA sequence | GenBank Accession NM_005314 |
| 10 | GRP receptor proteins sequence | GenBank Accession NM_005314 |
| 11 to 81 | Primers & probes for real-time PCR assay | This disclosure |
| 82-100 | Human telomeric repeats | U.S. Pat. No. 5,583,016 |
| 101 | Geron sequence designation GA_12064 | This disclosure |
| 102 | Geron sequence designation GA_23176 | This disclosure |
| 103 | Geron sequence designation GA_23468 | This disclosure |
| 104 | Geron sequence designation GA_23476 | This disclosure |
| 105 | Geron sequence designation GA_23484 | This disclosure |
| 106 | Geron sequence designation GA_23485 | This disclosure |
| 107 | Geron sequence designation GA_23486 | This disclosure |
| 108 | Geron sequence designation GA_23487 | This disclosure |
| 109 | Geron sequence designation GA_23488 | This disclosure |
| 110 | Geron sequence designation GA_23489 | This disclosure |
| 111 | Geron sequence designation GA_23490 | This disclosure |
| 112 | Geron sequence designation GA_23514 | This disclosure |
| 113 | Geron sequence designation GA_23515 | This disclosure |
| 114 | Geron sequence designation GA_23525 | This disclosure |
| 115 | Geron sequence designation GA_23572 | This disclosure |
| 116 | Geron sequence designation GA_23577 | This disclosure |
| 117 | Geron sequence designation GA_23579 | This disclosure |
| 118 | Geron sequence designation GA_23585 | This disclosure |
| 119 | Geron sequence designation GA_23596 | This disclosure |
| 120 | Geron sequence designation GA_23615 | This disclosure |
| 121 | Geron sequence designation GA_23634 | This disclosure |
| 122 | Geron sequence designation GA_23673 | This disclosure |
| 123 | Geron sequence designation GA_23683 | This disclosure |
| 124 | Geron sequence designation GA_23969 | This disclosure |
| 125 | Geron sequence designation GA_24037 | This disclosure |
| 126 | Geron sequence designation GA_32842 | This disclosure |
| 127 | Geron sequence designation GA_32860 | This disclosure |
| 128 | Geron sequence designation GA_32895 | This disclosure |
| 129 | Geron sequence designation GA_32913 | This disclosure |
| 130 | Geron sequence designation GA_32917 | This disclosure |
| 131 | Geron sequence designation GA_32926 | This disclosure |
| 132 | Geron sequence designation GA_32947 | This disclosure |
| 133 | Geron sequence designation GA_32979 | This disclosure |
| 134 | Geron sequence designation GA_32985 | This disclosure |
| 135 | Geron sequence designation GA_35405 | This disclosure |
| 136 | Geron sequence designation GA_38029 | This disclosure |
| 137 | Geron sequence designation GA_7542 | This disclosure |
| 138 | Geron sequence designation GA_8667 | This disclosure |
| 139 | Geron sequence designation GA_9014 | This disclosure |

| LOCUS | TERT 4015 bp mRNA linear PRI 31-OCT-2000 | SEQ. ID NO: 1 |
|---|---|---|
| DEFINITION | Homo sapiens telomerase reverse transcriptase (TERT), mRNA. | |
| ACCESSION | NM_003219 | |
| AUTHORS | Nakamura, T. M., Morin, G. B., Chapman, K. B., Weinrich, S. L., | |
| | Andrews, W. H., Lingner, J., Harley, C. B. and Cech, T. R. | |
| TITLE | Telomerase catalytic subunit homologs from fission yeast and human | |
| JOURNAL | Science 277 (5328), 955-959 (1997) | |
| CDS | 56..3454 | |
| LOCUS | POU5F1 1158 bp mRNA linear PRI 31-OCT-2000 | SEQ. ID NO: 3 |
| DEFINITION | Homo sapiens POU domain, class 5, transcription factor 1 (POU5F1), | |
| | mRNA. | |
| ACCESSION | NM_002701 | |
| AUTHORS | Takeda, J., Seino, S. and Bell, G. I. | |
| TITLE | Human Oct3 gene family: cDNA sequences, alternative splicing, gene | |
| | organization, chromosomal location, and expression at low levels in | |
| | adult tissues | |
| JOURNAL | Nucleic Acids Res. 20 (17), 4613-4620 (1992) | |
| CDS | 102..899 | |
| LOCUS | TDGF1 2033 bp mRNA linear PRI 05-NOV-2002 | SEQ. ID NO: 5 |
| DEFINITION | Homo sapiens teratocarcinoma-derived growth factor 1 (TDGF1), mRNA. | |
| ACCESSION | NM_003212 | |
| AUTHORS | Dono, R., Montuori, N., Rocchi, M., De Ponti-Zilli, L., Ciccodicola, A. | |
| | and Persico, M. G. | |
| TITLE | Isolation and characterization of the CRIPTO autosomal gene and its | |
| | X-linked related sequence | |
| JOURNAL | Am. J. Hum. Genet. 49 (3), 555-565 (1991) | |
| CDS | 248..814 | |
| LOCUS | PODXL 5869 bp mRNA linear PRI 01-NOV-2000 | SEQ. ID NO: 7 |
| DEFINITION | Homo sapiens podocalyxin-like (PODXL), mRNA. | |
| ACCESSION | NM_005397 | |
| AUTHORS | Kershaw, D. B., Beck, S. G., Wharram, B. L., Wiggins, J. E., Goyal, | |
| | M., Thomas, P. E. and Wiggins, R. C. | |
| TITLE | Molecular cloning and characterization of human podocalyxin-like | |
| | protein. Orthologous relationship to rabbit PCLP1 and rat | |
| | podocalyxin | |
| JOURNAL | J. Biol. Chem. 272 (25), 15708-15714 (1997) | |
| CDS | 251..1837 | |
| LOCUS | GRPR 1726 bp mRNA linear PRI 05-NOV-2002 | SEQ. ID NO: 9 |
| DEFINITION | Homo sapiens gastrin-releasing peptide receptor (GRPR), mRNA. | |
| ACCESSION | NM_005314 | |
| AUTHORS | Xiao, D., Wang, J , Hampton, L. L. and Weber, H. C. | |
| TITLE | The human gastrin-releasing peptide receptor gene structure, its | |
| | tissue expression and promoter | |
| JOURNAL | Gene 264 (1), 95-103 (2001) | |
| CDS | 399..1553 | |

| | |
|---|---|
| Bone Marrow Stromal antigen | |
| Forward primer: ACCTGCAACCACACTGTGATG | SEQ. ID NO: 11 |
| Probe: 6fam-CCCTAATGGCTTCCCTGGATGCAGA-tam | SEQ. ID NO: 12 |
| Reverse Primer: TTTCTTTTGTCCTTGGGCCTT | SEQ. ID NO: 13 |
| Podocalyxin-like | |
| Forward primer: GCTCGGCATATCAGTGAGATCA | SEQ. ID NO: 14 |
| Probe: 6fam-TCTCATCCGAAGCGCCCCCTG-tam | SEQ. ID NO: 15 |
| Reverse Primer: AGCTCGTCCTGAACCTCACAG | SEQ. ID NO: 16 |
| Rat GPC/glpican-2 (cerebroglycan) | |
| Forward primer: CTGGAAGAAATGTGGTCAGCG | SEQ. ID NO: 17 |
| Probe: 6fam-AGCGCTTAAGGTGCCGGTGTCTGAAG-tam | SEQ. ID NO: 18 |
| Reverse Primer: CATCAGAGCCTGGCTGCAG | SEQ. ID NO: 19 |
| Potassium channel subfamily k member 5 (TASK-2) | |
| Forward primer: ACCATCGGCTTCGGTGAC | SEQ. ID NO: 20 |
| Probe: 6fam-TGTGGCCGGTGTGAACCCCA-tam | SEQ. ID NO: 21 |
| Reverse Primer: TACAGGGCGTGGTAGTTGGC | SEQ. ID NO: 22 |
| Notch 1 protein | |
| Forward primer: TGAGAGCTTCTCCTGTGTCTGC | SEQ. ID NO: 23 |
| Probe: 6fam-CAAGGGCAGACCTGTGAGGTCGACA-tam | SEQ. ID NO: 24 |
| Reverse Primer: GGGCTCAGAACGCACTCGT | SEQ. ID NO: 25 |
| Teratocarcinoma-derived growth factor 1 (Cripto) | |
| Forward primer: TGAGCACGATGTGCGCA | SEQ. ID NO: 26 |
| Probe: 6fam-AGAGAACTGTGGGTCTGTGCCCCATG-tam | SEQ. ID NO: 27 |
| Reverse Primer: TTCTTGGGCAGCCAGGTG | SEQ. ID NO: 28 |
| Nel 1 like/NELL2 (Nel-like protein 2) | |
| Forward primer: CTTAAGTCGGCTCTTGCGTATGT | SEQ. ID NO: 29 |
| Probe: 6fam-ATGGCAAATGCTGTAAGGAATGCAAATCG-tam | SEQ. ID NO: 30 |
| Reverse Primer: AAGTAGGTTCGTCCTTGAAATTGG | SEQ. ID NO: 31 |
| Gastrin releasing peptide receptor | |
| Forward primer: CCGTGGAAGGGAATATACATGTC | SEQ. ID NO: 32 |
| Probe: 6fam-AGAAGCAGATTGAATCCCGGAAGCGA-TAM | SEQ. ID NO: 33 |
| Reverse Primer: CACCAGCACTGTCTTGGCAA | SEQ. ID NO: 34 |
| Bone morphogenetic protein receptor | |
| Forward primer: CAGATTATTGGGAGCCTATTTGTTC | SEQ. ID NO: 35 |
| Probe: 6fam-TCATTTCTCGTGTTCAAGGACAGAATCTGGAT-tam | SEQ. ID NO: 36 |
| Reverse Primer: CATCCCAGTGCCATGAAGC | SEQ. ID NO: 37 |
| ABC G2-ABC transporter | |
| Forward primer: GGCCTCAGGAAGACTTATGT | SEQ. ID NO: 38 |
| Probe: SYBR Green Detection Method | |
| Reverse Primer: AAGGAGGTGGTGTAGCTGAT | SEQ. ID NO: 39 |
| Solute carrier family 6, member 8 (SLC6A8) | |
| Forward primer: CCGGCAGCAT CAATGTCTG | SEQ. ID NO: 40 |
| Probe: 6fam-TCAAAGGCCTGGGCTACGCCTCC-tam | SEQ. ID NO: 41 |
| Reverse Primer: GTGTTGCAGTAGAAGACGATCACC | SEQ. ID NO: 42 |
| Oct 3/4 octamer-binding trasncription factor 3a (oct3a) (oct-4) | |
| Forward primer: GAAACCCACACTGCAGCAGA | SEQ. ID NO: 43 |
| Probe: 6fam-CAGCCACATCGCCCAGCAGC-TAM | SEQ. ID NO: 44 |
| Reverse Primer: CACATCCTTCTCGAGCCCA | SEQ. ID NO: 45 |
| Left-right determination factor b (LEFTB) | |
| Forward primer: TGCCGCCAGGAGATGTACA | SEQ. ID NO: 46 |
| Probe: 6fam-TGGGCCGAGAACTGGGTGCTG-tam | SEQ. ID NO: 47 |
| Reverse Primer: TCATAAGCCAGGAAGCCCG | SEQ. ID NO: 48 |
| Secreted phosphoprotein 1 (osteopontin) | |
| Forward primer: TTGCAGCCTTCTCAGCCAA | SEQ. ID NO: 49 |
| Probe: 6fam-CGCCGACCAAGGAAAACTCACTACCA-tam | SEQ. ID NO: 50 |
| Reverse Primer: GGAGGCAAAAGCAAATCACTG | SEQ. ID NO: 51 |
| Gamma-aminobutyric aci (GABA) A receptor, beta 3 | |
| Forward primer: CCGTCTGGTCTCGAGGAATG | SEQ. ID NO: 52 |
| Probe: 6fam-TCTTCGCCACAGGTGCCTATCCTCG-tam | SEQ. ID NO: 53 |
| Reverse Primer: TCAACCGAAAGCTCAGTGACA | SEQ. ID NO: 54 |
| Roundabout, axon guidance receptor, homologue 1 (ROBO1) | |
| Forward primer: GAGAGGAGGCGAAGCTGTCA | SEQ. ID NO: 55 |
| Probe: 6fam-CAGTGGAGGGAGGCCTGGACTTCTC-tam | SEQ. ID NO: 56 |
| Reverse Primer: GCGGCAGGTTCACTGATGT | SEQ. ID NO: 57 |
| Glucagon receptor | |
| Forward primer: CCACACAGACTACAAGTTCCGG | SEQ. ID NO: 58 |
| Probe: 6fam-TGGCCAAGTCCACGCTGACCCT-tam | SEQ. ID NO: 59 |
| Reverse Primer: CTTCGTGGACGCCCAGC | SEQ. ID NO: 60 |
| Leucine-rich PPR-motif hum 130 kda hum 130 kd leu | |
| Forward primer: GCAGCAGACCCCTTCTAGGTTAG | SEQ. ID NO: 61 |
| Probe: 6fam-ACCCGTGTCATCCAGGCATTGGC-tam | SEQ. ID NO: 62 |
| Reverse Primer: TGAACTACTTCTATGTTTTCAACATCACC | SEQ. ID NO: 63 |
| Thy-1 co-transcribed | |
| Forward primer: AGCCTCCAAGTCAGGTGGG | SEQ. ID NO: 64 |
| Probe: 6fam-CAGAGCTGCACAGGGTTTGGCCC-TAM | SEQ. ID NO: 65 |
| Reverse Primer: GGAGGAAGTGCCTCCCTTAGA | SEQ. ID NO: 66 |
| Solute carrier family 21 | |
| Forward primer: GCGTCACCTACCTGGATGAGA | SEQ. ID NO: 67 |
| Probe: 6fam-CCAGCTGCTCGCCCGTCTACATTG-tam | SEQ. ID NO: 68 |
| Reverse Primer: TGGCCGCTGTGTAGAAGATG | SEQ. ID NO: 69 |
| LY6H lympohocyte antigen 6 complex locus H | |
| Forward primer: CGAATCACCGATCCCAGC | SEQ. ID NO: 70 |
| Probe: 6fam-CAGCAGGAAGGATCACTCGGTGAACAA-tam | SEQ. ID NO: 71 |
| Reverse Primer: CGAAGTCACAGGAGGAGGCA | SEQ. ID NO: 72 |
| Plexin (PLXNB3) | |
| Forward primer: GAGAAGGTGTTGGACCAAGTCTACA | SEQ. ID NO: 73 |
| Probe: 6fam-CCTCAGTGCATGCCCTAGACCTTGAGTG-tam | SEQ. ID NO: 74 |
| Reverse Primer: CTTCGTCCGATAGGGTCAGG | SEQ. ID NO: 75 |
| ICAM | |
| Forward primer: ACTCCAGAACGGGTGGAACTG | SEQ. ID NO: 76 |
| Probe: 6fam-ACCCCTCCCCTCTTGGCAGCC-tam | SEQ. ID NO: 77 |
| Reverse Primer: CGTAGGGTAAGGTTCTTGCCC | SEQ. ID NO: 78 |
| Rhodopsin | |
| Forward primer: CCGGCTGGTCCAGGTACAT | SEQ. ID NO: 79 |
| Probe: 6fam-CCGAGGGCCTGCAGTGCTCG-tam | SEQ. ID NO: 80 |
| Reverse Primer: TTGAGCGTGTAGTAGTCGATTCCA | SEQ. ID NO: 81 |

The subject matter provided in this disclosure can be modified as a matter of routine optimization, without departing from the spirit of the invention, or the scope of the appended claims.

The claimed invention is:

1. A method for assessing a culture of undifferentiated primate pluripotent stem (pPS) cells or their progeny, comprising detecting or measuring expression of two or more of the markers in any of Tables 5 to 9, other than hTERT or Oct 3/4.

2. The method of claim 1, comprising measuring expression of two or more of the markers in Tables 2, 7, and 9(C), and correlating the expression measured with the presence of undifferentiated embryonic stem (ES) cells in the culture.

3. The method of claim 1, comprising measuring expression of two or more of the markers in Tables 3 and 8, and correlating the expression measured with the presence of differentiated cells in the culture.

4. The method of claim 1, comprising detecting or measuring expression of one or more of the following markers: bone marrow stromal antigen; Podocalyxin-like; Rat GPC/glypican-2 (cerebroglycan); Potassium channel subfamily k member 5 (TASK-2); Notch 1 protein; Teratocarcinoma-derived growth factor 1 (Cripto); Nel 1 like/NELL2 (Nel-like protein 2); Gastrin releasing peptide receptor; Bone morphogenetic protein; ABCG2- ABC transporter; Solute carrier family 6, member 8 (SLC6A8); hTERT; Oct 3/4 Octamer-binding transcription factor 3a (Oct-3a) (Oct-4); Left-right determination factor b (LEFT); Secreted phosphoprotein 1 (osteopontin); Gamma-aminobutyric acid (GABA) A receptor, beta 3; Roundabout, axon guidance receptor, homologue 1 (ROBO1); Glucagon receptor; Leucine-rich ppr-motif hum 130 kDa hum130leu 130 kd leu; Thy-1 co-transcribed; Solute carrier family 21; LY6H lymphocyte antigen 6 complex locus H; Plexin (PLXNB3); Armadillo repeat protein deleted in velo-cardio-facial syndrome; and Ephrin type-a receptor 1 (EPHA1).

5. The method of claim 1, comprising detecting or measuring expression of three or more of said markers.

6. The method of claim 1 further comprising detecting or measuring expression of hTERT and/or Oct 3/4.

7. A method for assessing a culture of undifferentiated primate pluripotent stem (pPS) cells or their progeny, comprising detecting or measuring:
a marker from the following list: Cripto, gastrin-releasing peptide (GRP) receptor, and podocalyxin-like protein; and
either hTERT and/or Oct 3/4, or a second marker from the list.

8. The method of claim 7, comprising detecting or measuring at least two markers from the list.

9. The method of claim 7, comprising detecting or measuring at least two markers from the list, and detecting or measuring hTERT and/or Oct 3/4.

10. The method of claim 7, comprising detecting or measuring Cripto, gastrin-releasing peptide (GRP) receptor podocalyxin-like protein, hTERT, and Oct 3/4.

11. The method of claim 1, wherein expression of the marker(s) is detected or measured at the mRNA level by PCR amplification.

12. The method of claim 1, wherein expression of the marker(s) is detected or measured at the protein or enzyme product level by antibody assay.

13. The method of claim 1, comprising quantifying the proportion of undifferentiated pPS cells or differentiated cells in the culture from said marker expression.

14. The method of claim 1, comprising assessing the ability of a culture system or component thereof to maintain pPS cells in an undifferentiated state from said marker expression.

15. The method of claim 14, comprising assessing the ability of a soluble factor to maintain pPS cells in an undifferentiated state from said marker expression.

16. The method of claim 14, comprising assessing the ability of a culture medium to maintain pPS cells in an undifferentiated state from said marker expression.

17. The method of claim 14, comprising assessing the ability of a preparation of feeder cells to maintain pPS cells in an undifferentiated state from said marker expression.

18. The method of claim 1, comprising assessing the ability of a culture system or component thereof to cause differentiation of pPS cells into a culture of lineage-restricted precursor cells and/or terminally differentiated cells.

19. The method of claim 1, comprising assessing the suitability of a pPS cell culture for preparing cells for human administration.

20. The method of claim 7, wherein the level of the marker is determined to be at least 100-fold higher than the level of the marker in BJ fibroblasts.

21. A method for assessing the growth characteristics of a cell population, comprising detecting or measuring expression of two or more of the markers in any of Tables 5 to 9, at least one of which is neither hTERT nor Oct 3/4.

22. The method of claim 21, comprising detecting or measuring:
a marker from the following list: Cripto, gastrin-releasing peptide (GRP) receptor, and podocalyxin-like protein; and
either hTERT and/or Oct 3/4, or a second marker from the list.

23. The method of claim 21, wherein the cell population has been obtained by culturing cells from a human blastocyst.

24. The method of claim 23, comprising determining whether the cell population is pluripotent from said marker expression.

25. The method of claim 21, wherein the cell population has been obtained from a human patient suspected of having a clinical condition related to abnormal cell growth.

26. The method of claim 25, comprising assessing whether the patient has a malignancy from said marker expression.

27. A method for maintaining pPS cells in a pluripotent state, comprising causing them to express one of the following markers at a higher level:
Forkhead box O1A (FOXO1A); Zic family member 3 (ZIC3); Hypothetical protein FLJ20582; Forkhead box H1 (FOXH1); Zinc finger protein, Hsal2; KRAB-zinc finger protein SZF1-1; or Zinc finger protein of cerebellum ZIC2; or
any other marker listed in Table 5 with the symbol “\mathring{x}”.

28. The method of claim 27, wherein the cells are caused to express the marker by genetically altering it with a gene that encodes the marker.

29. A method for causing pPS cells to differentiate into a particular tissue type, comprising causing them to express one of the following markers at a lower level:
Forkhead box O1A (FOX01A); Zic family member 3 (ZIC3); Hypothetical protein FLJ20582; Forkhead box H1 (FOXH1); Zinc finger protein, Hsal2; KRAB-zinc finger protein SZF1-1; or Zinc finger protein of cerebellum ZIC2; or
any other maker listed in Table 5 with the symbol “\mathring{x}”;
or by causing them to express one of the markers listed in Table 6 with the symbol “\mathring{x}” at a higher level.

30. The method of claim 29, wherein the cells are caused to express the marker by genetically altering it with a gene that encodes the marker.

31. A method for maintaining pPS cells in a pluripotent state, comprising culturing pPS cells or their progeny in the presence of a normally secreted protein that is encoded by a gene listed in Table 2, 5, 7, or 9.

32. A method for causing pPS cells to differentiate, comprising culturing pPS cells or their progeny in the presence of a normally secreted protein that is encoded by a gene listed in Table 3, 6, or 8.

33. A method for causing an encoding sequence to be preferentially expressed in undifferentiated pPS cells, comprising genetically altering pPS cells with the encoding sequence under control of a promoter for one of the markers listed in Table 2, 5, or 7.

34. The method of claim 33, further comprising selecting undifferentiated cells, wherein the encoding sequence is a reporter gene (such as a gene that causes the cells to emit fluorescence), or a positive selection marker (such as a drug resistance gene).

35. The method of claim 33, further comprising depleting undifferentiated cells from a population of differentiated cells, wherein the encoding sequence is a negative selection marker (such as a gene that activates apoptosis or converts a prodrug into a compound that is toxic to the cell).

36. A method for causing an encoding sequence to be preferentially expressed in differentiated cells, comprising genetically altering the pPS cells with the encoding sequence under control of a promoter for one of the markers listed in Table 3, 6, or 8.

37. The method of claim 36, further comprising selecting differentiated cells, wherein the encoding sequence is a reporter gene (such as a gene that causes the cells to emit fluorescence), or a positive selection marker (such as a drug resistance gene).

38. The method of claim 36, further comprising depleting differentiated cells from a population of undifferentiated cells, wherein the encoding sequence is a negative selection marker (such as a gene that activates apoptosis or converts a prodrug into a compound that is lethal to the cell).

39. A method for sorting differentiated cells from less differentiated cells, comprising separating cells expressing a surface marker in any of Tables 5 to 9 from cells not expressing the marker.

40. The method of claim 39, wherein the cells are sorted using an antibody or lectin that binds the marker or product thereof on the cell surface.

41. A method for causing pPS cells to proliferate without differentiation, comprising culturing them in a culture system assessed according to the method of claim 6.

42. A method for causing pPS cells to proliferate without differentiation, comprising culturing them with mesenchymal stem cells.

43. A method for identifying genes that are up- or down-regulated during differentiation of pPS cells, comprising:
a) sequencing transcripts in an expression library from undifferentiated pPS cells;
b) sequencing transcripts in one or more expression libraries from one or more cell types that have differentiated from the same line of pPS cells;
c) determining the frequency of transcripts from each gene sequenced in each of the libraries; and
d) identifying the gene as being up- or down-regulated during differentiation of the pPS cells if the frequency of transcripts in the library from the undifferentiated pPS cells is statistically different from the frequency of transcripts in one or more libraries from the differentiated cell types.

44. The method of claim 43, further comprising assessing a culture of pPS cells depending on the expression level in the culture of a marker identified in step d).

45. A kit for assessing a culture of pPS cells according to claim 1, comprising polynucleotide probes and/or primers for specifically amplifying a transcript for two or more markers in any of Tables 5 to 9, accompanied by written instructions for assessing the pPS cells or their progeny according to the expression of said markers measured using the probes or primers in the kit.

46. A kit for assessing a culture of pPS cells according to claim 1, comprising antibodies specific for each gene product of two or more markers in any of Tables 5 to 9, accompanied by written instructions for assessing the pPS cells or their progeny according to the expression of said markers measured using the antibodies in the kit.

47. The method of claim 1, wherein the pPS cells are obtained from a human blastocyst, or are the progeny of such cells.

48. The method of claim 1, wherein the pPS cells are human embryonic stem cells.


SEQUENCE LISTING (139 sequences)

SEQ ID NO: 1 - DNA, 4015 bp, Homo sapiens
  CDS (56)..(3454)
gcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 2 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - DNA, 1158 bp, Homo sapiens
  CDS (102)..(899)
gtagtcctttgttacatgcatgagtcagtgaacagggaatgggtgaatgacatttgtgggtaggttatttctagaagttaggtgggcagctcggaaggcagatgcacttctacagactattccttggggccacacgtaggttcttgaatcccgaatggaaaggggagattgataactggtgtgtttatgttcttacaagtcttctgccttttaaaatccagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtgccccaggctatgggagccctcacttcactgcactgtactcctcggtccctttccctgagggggaagcctttccccctgtctctgtcaccactctgggctctcccttgcattcaaactgaggtgcctgcctgcccttctaggaatgggggacagggggaggggaggagctagggaaagaaaacctggagtttgtgccagggtttttggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcttgggacacagtagataga

SEQ ID NO: 4 - protein, 265 aa, Homo sapiens
MHFYRLFLGATRRFLNPEWKGEIDNWCVYVLTSLLPFKIQSQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGAPGYGSPHFTALYSSVPFPEGEAFPPVSVTTLGSPLHSN

SEQ ID NO: 5 - DNA, 2033 bp, Homo sapiens
  CDS (248)..(814)
ggagaatccccggaaaggctgagtctccagctcaaggtcaaaacgtccaaggccgaaagccctccagtttcccctggacgccttgctcctgcttctgctacgaccttctggggaaaacgaatttctcattttcttcttaaattgccattttcgctttaggagatgaatgttttcctttggctgttttggcaatgactctgaattaaagcgatgctaacgcctcttttccccctaattgttaaaagctatggactgcaggaagatggcccgcttctcttacagtgtgatttggatcatggccatttctaaagtctttgaactgggattagttgccgggctgggccatcaggaatttgctcgtccatctcggggatacctggccttcagagatgacagcatttggccccaggaggagcctgcaattcggcctcggtcttcccagcgtgtgccgcccatggggatacagcacagtaaggagctaaacagaacctgctgcctgaatgggggaacctgcatgctggggtccttttgtgcctgccctccctccttctacggacggaactgtgagcacgatgtgcgcaaagagaactgtgggtctgtgccccatgacacctggctgcccaagaagtgttccctgtgtaaatgctggcacggtcagctccgctgctttcctcaggcatttctacccggctgtgatggccttgtgatggatgagcacctcgtggcttccaggactccagaactaccaccgtctgcacgtactaccacttttatgctagttggcatctgcctttctatacaaagctactattaatcgacattgacctatttccagaaatacaattttagatatcatgcaaatttcatgaccagtaaaggctgctgctacaatgtcctaactgaaagatgatcatttgtagttgccttaaaataatgaatacaatttccaaaatggtctctaacatttccttacagaactacttcttacttctttgccctgccctctcccaaaaaactacttcttttttcaaaagaaagtcagccatatctccattgtgcctaagtccagtgtttctttttttttttttttttgagacggagtctcactctgtcacccaggctggactgcaatgacgcgatcttggttcactgcaacctccgcatccggggttcaagccattctcctgcctaagcctcccaagtaactgggattacaggcatgtgtcaccatgcccagctaatttttttgtattttagtagagatgggggtttcaccatattggccagtctggtctcgaactctgaccttgtgatccatcgatcagcctctcgagtgctgagattacacacgtgagcaactgtgcaaggcctggtgtttcttgatacatgtaattctaccaaggtcttcttaatatgttcttttaaatgattgaattatatgttcagattattggagactaattctaatgtggaccttagaatacagttttgagtagagttgatcaaaatcaattaaaatagtctctttaaaaggaaagaaaacatctttaaggggaggaaccagagtgctgaaggaatggaagtccatctgcgtgtgtgcagggagactgggtaggaaagaggaagcaaatagaagagagaggttgaaaaacaaaatgggttacttgattggtgattaggtggtggtagagaagcaagtaaaaaggctaaatggaagggcaagtttccatcatctatagaaagctatataagacaagaactcccctttttttcccaaaggcattataaaaagaatgaagcctccttagaaaaaaaattatacctcaatgtccccaacaagattgcttaataaattgtgtttcctccaagctattcaattcttttaactgttgtagaagacaaaatgttcacaatatatttagttgtaaaccaagtgatcaaactacatattgtaaagcccatttttaaaatacattgtatatatgtgtatgcacagtaaaaatggaaactatattgacctaaaaaaaaaaaaa

SEQ ID NO: 6 - protein, 188 aa, Homo sapiens
MDCRKMARFSYSVIWIMAISKVFELGLVAGLGHQEFARPSRGYLAFRDDSIWPQEEPAIRPRSSQRVPPMGIQHSKELNRTCCLNGGTCMLGSFCACPPSFYGRNCEHDVRKENCGSVPHDTWLPKKCSLCKCWHGQLRCFPQAFLPGCDGLVMDEHLVASRTPELPPSARTTTFMLVGICLSIQSYY

SEQ ID NO: 7 - DNA, 5869 bp, Homo sapiens
  CDS (251)..(1837)
aaacgccgcccaggacgcagccgccgccgccgccgctcctctgccactggctctgcgccccagcccggctctgctgcagcggcagggaggaagagccgccgcagcgcgactcgggagccccgggccacagcctggcctccggagccacccacaggcctccccgggcggcgcccacgctcctaccgcccggacgcgcggatcctccgccggcaccgcagccacctgctcccggcccagaggcgacgacacgatgcgctgcgcgctggcgctctcggcgctgctgctactgttgtcaacgccgccgctgctgccgtcgtcgccgtcgccgtcgccgtcgccgtcgccctcccagaatgcaacccagactactacggactcatctaacaaaacagcaccgactccagcatccagtgtcaccatcatggctacagatacagcccagcagagcacagtccccacttccaaggccaacgaaatcttggcctcggtcaaggcgaccacccttggtgtatccagtgactcaccggggactacaaccctggctcagcaagtctcaggcccagtcaacactaccgtggctagaggaggcggctcaggcaaccctactaccaccatcgagagccccaagagcacaaaaagtgcagacaccactacagttgcaacctccacagccacagctaaacctaacaccacaagcagccagaatggagcagaagatacaacaaactctggggggaaaagcagccacagtgtgaccacagacctcacatccactaaggcagaacatctgacgacccctcaccctacaagtccacttagcccccgacaacccactttgacgcatcctgtggccaccccaacaagctcgggacatgaccatcttatgaaaatttcaagcagttcaagcactgtggctatccctggctacaccttcacaagcccggggatgaccaccaccctaccgtcatcggttatctcgcaaagaactcaacagacctccagtcagatgccagccagctctacggccccttcctcccaggagacagtgcagcccacgagcccggcaacggcattgagaacacctaccctgccagagaccatgagctccagccccacagcagcatcaactacccaccgataccccaaaacaccttctcccactgtggctcatgagagtaactgggcaaagtgtgaggatcttgagacacagacacagagtgagaagcagctcgtcctgaacctcacaggaaacaccctctgtgcagggggcgcttcggatgagaaattgatctcactgatatgccgagcagtcaaagccaccttcaacccggcccaagataagtgcggcatacggctggcatctgttccaggaagtcagaccgtggtcgtcaaagaaatcactattcacactaagctccctgccaaggatgtgtacgagcggctgaaggacaaatgggatgaactaaaggaggcaggggtcagtgacatgaagctaggggaccaggggccaccggaggaggccgaggaccgcttcagcatgcccctcatcatcaccatcgtctgcatggcgtcattcctgctcctcgtggcggccctctatggctgctgccaccagcgcctctcccagaggaaggaccagcagcggctaacagaggagctgcagacagtggagaatggttaccatgacaacccaacactggaagtgatggagacctcttctgagatgcaggagaagaaggtggtcagcctcaacggggagctgggggacagctggatcgtccctctggacaacctgaccaaggacgacctggatgaggaggaagacacacacctctagtccggtctgccggtggcctccagcagcaccacagagctccagaccaaccaccccaagtgccgtttggatggggaagggaaagactggggagggagagtgaactccgaggggtgtcccctcccaatccccccagggccttaatttttcccttttcaacctgaacaaatcacattctgtccagattcctcttgtaaaataacccactagtgcctgagctcagtgctgctggatgatgagggagatcaagaaaaagccacgtaagggactttatagatgaactagtggaatcccttcattctgcagtgagattgccgagacctgaagagggtaagtgacttgcccaaggtcagagccacttggtgacagagccaggatgagaacaaagattccatttgcaccatgccacactgctgtgttcacatgtgccttccgtccagagcagtcccgggcaggggtgaaactccagcaggtggctgggctggaaaggagggcagggctacatcctggctcggtgggatctgacgacctgaaagtccagctcccaagttttccttctcctaccccagcctcgtgtacccatcttcccaccctctatgttcttacccctccctacactcagtgtttgttcccacttactctgtcctggggcctctgggattagcacaggttattcataaccttgaaccccttgttctggattcggattttctcacatttgcttcgtgagatgggggcttaacccacacaggtctccgtgcgtgaaccaggtctgcttaggggacctgcgtgcaggtgaggagagaaggggacactcgagtccaggctggtatctcagggcagctgatgaggggtcagcaggaacactggcccattgcccctggcactccttgcagaggccacccacgatcttctttgggcttccatttccaccagggactaaaatctgctgtagctagtgagagcagcgtgttccttttgttgttcactgctcagctgatgggagtgattccctgagacccagtatgaaagagcagtggctgcaggagaggccttcccggggccccccatcagcgatgtgtcttcagagacaatccattaaagcagccaggaaggacaggctttcccctgtatatcataggaaactcagggacatttcaagttgctgagagttttgttatagttgttttctaacccagccctccactgccaaaggccaaaagctcagacagttggcagacgtccagttagctcatctcactcactctgattctcctgtgccacaggaaaagagggcctggaaagcgcagtgcatgctgggtgcatgaagggcagcctgggggacagactgttgtgggaacgtcccactgtcctggcctggagctaggccttgctgttcctcttctctgtgagcctagtggggctgctgcggttctcttgcagtttctggtggcatctcaggggaacacaaaagctatgtctattccccaatataggacttttatgggctcggcagttagctgccatgtagaaggctcctaagcagtgggcatggtgaggtttcatctgattgagaagggggaatcctgtgtggaatgttgaactttcgccatggtctccatcgttctgggcgtaaattccctgggatcaagtaggaaaatgggcagaactgcttaggggaatgaaattgccatttttcgggtgaaacgccacacctccagggtcttaagagtcaggctccggctgtagtagctctgatgaaataggctatccactcgggatggcttactttttaaaagggtagggggaggggctggggaagatctgtcctgcaccatctgcctaattccttcctcacagtctgtagccatctgatatcctagggggaaaaggaaggccaggggttcacatagggccccagcgagtttcccaggagttagagggatgcgaggctaacaagttccaaaaacatctgccccgatgctctagtgtttggaggtgggcaggatggagaacagtgcctgtttgggggaaaacaggaaatcttgttaggcttgagtgaggtgtttgcttccttcttgcccagcgctgggttctctccacccagtaggttttctgttgtggtcccgtgggagaggccagactggattattcctcctttgctgatcctgggtcacacttcaccagccagggcttttgacggagacagcaaataggcctctgcaaatcaatcaaaggctgcaaccctatggcctcttggagacagatgatgactggcaaggactagagagcaggagtgcctggccaggtcggtcctgactctcctgactctccatcgctctgtccaaggagaacccggagaggctctgggctgattcagaggttactgctttatattcgtccaaactgtgttagtctaggcttaggacagcttcagaatctgacaccttgccttgctcttgccaccaggacacctatgtcaacaggccaaacagccatgcatctataaaggtcatcatcttctgccacctttactgggttctaaatgctctctgataattcagagagcattgggtctgggaagaggtaagaggaacactagaagctcagcatgacttaaacaggttgtagcaaagacagtttatcatcaactctttcagtggtaaactgtggtttccccaagctgcacaggaggccagaaaccacaagtatgatgactaggaagcctactgtcatgagagtggggagacaggcagcaaagcttatgaaggaggtacagaatattctttgcgttgtaagacagaatacgggtttaatctagtctaggcrccagatttttttcccgcttgataaggaaagctagcagaaagtttatttaaaccacttcttgagctttatcttttttgacaatatactggagaaactttgaagaacaagttcaaactgatacatatacacatatttttttgataatgtaaatacagtgaccatgttaacctaccctgcactgctttaagtgaacatactttgaaaaagcattatgttagctgagtgatggccaagttttttctctggacaggaatgtaaatgtcttactggaaatgacaagtttttgcttgatttttttttttaaacaaaaaatgaaatataacaagacaaacttatgataaagtatttgtcttgtagatcaggtgttttgttttgtttttttaattttaaaatgcaaccctgccccctccccagcaaagtcacagctccatttcagtaaaggttggagtcaatatgctctggttggcaggcaaccctgtagtcatggagaaaggtatttcaagatctagtccaatctttttctagagaaaaagataatctgaagctcacaaagatgaagtgacttcctcaaaatcacatggttcaggacagaaacaagattaaaacctggatccacagactgtgcgcctcagaaggaataatcggtaaattaagaattgctactcgaaggtgccagaatgacacaaaggacagaattcctttcccagttgttaccctagcaaggctagggagggcatgaacacaaacataagaactggtcttctcacactttctctgaatcatttaggtttaagatgtaagtgaacaattctttctttctgccaagaaacaaagttttggatgagcttttatatatggaacttactccaacaggactgagggaccaaggaaacatgatgggggaggcaagagagggcaaagagtaaaactgtagcatagcttttgtcacggtcactagctgatccctcaggtctgctgcaaacacagcatggaggacacagatgactctttggtgttggtctttttgtctgcagtgaatgttcaacagtttgcccaggaactgggggatcatatatgtcttagtggacaggggtctgaagtacactggaatttactgagaaacttgtttgtaaaaactatagttaataattattgcattttcttacaaaaatatattttggaaaattgtatactgtcaattaaagt

SEQ ID NO: 8 - protein, 528 aa, Homo sapiens
MRCALALSALLLLLSTPPLLPSSPSPSPSPSPSQNATQTTTDSSNKTAPTPASSVTIMATDTAQQSTVPTSKANEILASVKATTLGVSSDSPGTTTLAQQVSGPVNTTVARGGGSGNPTTTIESPKSTKSADTTTVATSTATAKPNTTSSQNGAEDTTNSGGKSSHSVTTDLTSTKAEHLTTPHPTSPLSPRQPTLTHPVATPTSSGHDHLMKISSSSSTVAIPGYTFTSPGMTTTLPSSVISQRTQQTSSQMPASSTAPSSQETVQPTSPATALRTPTLPETMSSSPTAASTTHRYPKTPSPTVAHESNWAKCEDLETQTQSEKQLVLNLTGNTLCAGGASDEKLISLICRAVKATFNPAQDKCGIRLASVPGSQTVVVKEITIHTKLPAKDVYERLKDKWDELKEAGVSDMKLGDQGPPEEAEDRFSMPLIITIVCMASFLLLVAALYGCCHQRLSQRKDQQRLTEELQTVENGYHDNPTLEVMETSSEMQEKKVVSLNGELGDSWIVPLDNLTKDDLDEEEDTHL

SEQ ID NO: 9 - DNA, 1726 bp, Homo sapiens
  CDS (399)..(1553)
ccagattctaaatatcaggaaagacgctgtgggaaaatagcaggccaaaagttcttagtaaactgcagccagggagactcagactagaatggaggtagaaagaactgatgcagagtgggtttaattctaagcctttttgtggctaagttttgttgttgttaacttattgaatttagagttgtattgcactggtcatgtgaaagccagagcagcaccagtgtcaaaatagtgacagagagttttgaataccatagttagtatatatgtactcagagtatttttattaaagaaggcaaagagcccggcatagatcttatcttcatcttcactcggttgcaaaatcaatagttaagaaatagcatctaagggaacttttaggtgggaaaaaaaatctagagatggctctaaatgactgtttccttctgaacttggaggtggaccatttcatgcactgcaacatctccagtcacagtgcggatctccccgtgaacgatgactggtcccacccggggatcctctatgtcatccctgcagtttatggggttatcattctgataggcctcattggcaacatcactttgatcaagatcttctgtacagtcaagtccatgcgaaacgttccaaacctgttcatttccagtctggctttgggagacctgctcctcctaataacgtgtgctccagtggatgccagcaggtacctggctgacagatggctatttggcaggattggctgcaaactgatcccctttatacagcttacctctgttggggtgtctgtcttcacactcacggcgctctcggcagacagatacaaagccattgtccggccaatggatatccaggcctcccatgccctgatgaagatctgcctcaaagccgcctttatctggatcatctccatgctgctggccattccagaggccgtgttttctgacctccatcccttccatgaggaaagcaccaaccagaccttcattagctgtgccccatacccacactctaatgagcttcaccccaaaatccattctatggcttcctttctggtcttctacgtcatcccactgtcgatcatctctgtttactactacttcattgctaaaaatctgatccagagtgcttacaatcttcccgtggaagggaatatacatgtcaagaagcagattgaatcccggaagcgacttgccaagacagtgctggtgtttgtgggcctgttcgccttctgctggctccccaatcatgtcatctacctgtaccgctcctaccactactctgaggtggacacctccatgctccactttgtcaccagcatctgtgcccgcctcctggccttcaccaactcctgcgtgaacccctttgccctctacctgctgagcaagagtttcaggaaacagttcaacactcagctgctctgttgccagcctggcctgatcatccggtctcacagcactggaaggagtacaacctgcatgacctccctcaagagtaccaacccctccgtggccacctttagcctcatcaatggaaacatctgtcacgagcggtatgtctagattgacccttgattttgccccctgagggacggttttgctttatggctagacaggaacccttgcatccattgttgtgtctgtgccctccaaagagccttcagaatgctcctgagtggtgtaggtgggggtggggaggcccaaatgatggatcaccattatattttgaaagaagc

SEQ ID NO: 10 - protein, 384 aa, Homo sapiens
MALNDCFLLNLEVDHFMHCNISSHSADLPVNDDWSHPGILYVIPAVYGVIILIGLIGNITLIKIFCTVKSMRNVPNLFISSLALGDLLLLITCAPVDASRYLADRWLFGRIGCKLIPFIQLTSVGVSVFTLTALSADRYKAIVRPMDIQASHALMKICLKAAFIWIISMLLAIPEAVFSDLHPFHEESTNQTFISCAPYPHSNELHPKIHSMASFLVFYVIPLSIISVYYYFIAKNLIQSAYNLPVEGNIHVKKQIESRKRLAKTVLVFVGLFAFCWLPNHVIYLYRSYHYSEVDTSMLHFVTSICARLLAFTNSCVNPFALYLLSKSFRKQFNTQLLCCQPGLIIRSHSTGRSTTCMTSLKSTNPSVATFSLINGNICHERYV

SEQ ID NO: 11 - DNA, 21 bp, Homo sapiens
acctgcaaccacactgtgatg

SEQ ID NO: 12 - DNA, 25 bp, Homo sapiens
ccctaatggcttccctggatgcaga

SEQ ID NO: 13 - DNA, 21 bp, Homo sapiens
tttcttttgtccttgggcctt

SEQ ID NO: 14 - DNA, 22 bp, Homo sapiens
gctcggcatatcagtgagatca

SEQ ID NO: 15 - DNA, 21 bp, Homo sapiens
tctcatccgaagcgccccctg

SEQ ID NO: 16 - DNA, 21 bp, Homo sapiens
agctcgtcctgaacctcacag

SEQ ID NO: 17 - DNA, 21 bp, Homo sapiens
ctggaagaaatgtggtcagcg

SEQ ID NO: 18 - DNA, 26 bp, Homo sapiens
agcgcttaaggtgccggtgtctgaag

SEQ ID NO: 19 - DNA, 19 bp, Homo sapiens
catcagagcctggctgcag

SEQ ID NO: 20 - DNA, 18 bp, Homo sapiens
accatcggcttcggtgac

SEQ ID NO: 21 - DNA, 20 bp, Homo sapiens
tgtggccggtgtgaacccca

SEQ ID NO: 22 - DNA, 20 bp, Homo sapiens
tacagggcgtggtagttggc

SEQ ID NO: 23 - DNA, 22 bp, Homo sapiens
tgagagcttctcctgtgtctgc

SEQ ID NO: 24 - DNA, 25 bp, Homo sapiens
caagggcagacctgtgaggtcgaca

SEQ ID NO: 25 - DNA, 19 bp, Homo sapiens
gggctcagaacgcactcgt

SEQ ID NO: 26 - DNA, 17 bp, Homo sapiens
tgagcacgatgtgcgca

SEQ ID NO: 27 - DNA, 26 bp, Homo sapiens
agagaactgtgggtctgtgccccatg

SEQ ID NO: 28 - DNA, 18 bp, Homo sapiens
ttcttgggcagccaggtg

SEQ ID NO: 29 - DNA, 23 bp, Homo sapiens
cttaagtcggctcttgcgtatgt

SEQ ID NO: 30 - DNA, 29 bp, Homo sapiens
atggcaaatgctgtaaggaatgcaaatcg

SEQ ID NO: 31 - DNA, 24 bp, Homo sapiens
aagtaggttcgtccttgaaattgg

SEQ ID NO: 32 - DNA, 23 bp, Homo sapiens
ccgtggaagggaatatacatgtc

SEQ ID NO: 33 - DNA, 26 bp, Homo sapiens
agaagcagattgaatcccggaagcga

SEQ ID NO: 34 - DNA, 20 bp, Homo sapiens
caccagcactgtcttggcaa

SEQ ID NO: 35 - DNA, 25 bp, Homo sapiens
cagattattgggagcctatttgttc

SEQ ID NO: 36 - DNA, 32 bp, Homo sapiens
tcatttctcgtgttcaaggacagaatctggat

SEQ ID NO: 37 - DNA, 19 bp, Homo sapiens
catcccagtgccatgaagc

SEQ ID NO: 38 - DNA, 20 bp, Homo sapiens
ggcctcaggaagacttatgt

SEQ ID NO: 39 - DNA, 20 bp, Homo sapiens
aaggaggtggtgtagctgat

SEQ ID NO: 40 - DNA, 19 bp, Homo sapiens
ccggcagcatcaatgtctg

SEQ ID NO: 41 - DNA, 23 bp, Homo sapiens
tcaaaggcctgggctacgcctcc

SEQ ID NO: 42 - DNA, 24 bp, Homo sapiens
gtgttgcagtagaagacgatcacc

SEQ ID NO: 43 - DNA, 20 bp, Homo sapiens
gaaacccacactgcagcaga

SEQ ID NO: 44 - DNA, 20 bp, Homo sapiens
cagccacatcgcccagcagc

SEQ ID NO: 45 - DNA, 19 bp, Homo sapiens
cacatccttctcgagccca

SEQ ID NO: 46 - DNA, 19 bp, Homo sapiens
tgccgccaggagatgtaca

SEQ ID NO: 47 - DNA, 21 bp, Homo sapiens
tgggccgagaactgggtgctg

SEQ ID NO: 48 - DNA, 19 bp, Homo sapiens
tcataagccaggaagcccg

SEQ ID NO: 49 - DNA, 19 bp, Homo sapiens
ttgcagccttctcagccaa

SEQ ID NO: 50 - DNA, 26 bp, Homo sapiens
cgccgaccaaggaaaactcactacca

SEQ ID NO: 51 - DNA, 21 bp, Homo sapiens
ggaggcaaaagcaaatcactg

SEQ ID NO: 52 - DNA, 20 bp, Homo sapiens
ccgtctggtctcgaggaatg

SEQ ID NO: 53 - DNA, 25 bp, Homo sapiens
tcttcgccacaggtgcctatcctcg

SEQ ID NO: 54 - DNA, 21 bp, Homo sapiens
tcaaccgaaagctcagtgaca

SEQ ID NO: 55 - DNA, 20 bp, Homo sapiens
gagaggaggcgaagctgtca

SEQ ID NO: 56 - DNA, 25 bp, Homo sapiens
cagtggagggaggcctggacttctc

SEQ ID NO: 57 - DNA, 19 bp, Homo sapiens
gcggcaggttcactgatgt

SEQ ID NO: 58 - DNA, 22 bp, Homo sapiens
ccacacagactacaagttccgg

SEQ ID NO: 59 - DNA, 22 bp, Homo sapiens
tggccaagtccacgctgaccct

SEQ ID NO: 60 - DNA, 17 bp, Homo sapiens
cttcgtggacgcccagc

SEQ ID NO: 61 - DNA, 23 bp, Homo sapiens
gcagcagaccccttctaggttag

SEQ ID NO: 62 - DNA, 23 bp, Homo sapiens
acccgtgtcatccaggcattggc

SEQ ID NO: 63 - DNA, 29 bp, Homo sapiens
tgaactacttctatgttttcaacatcacc

SEQ ID NO: 64 - DNA, 19 bp, Homo sapiens
agcctccaagtcaggtggg

SEQ ID NO: 65 - DNA, 23 bp, Homo sapiens
cagagctgcacagggtttggccc

SEQ ID NO: 66 - DNA, 21 bp, Homo sapiens
ggaggaagtgcctcccttaga

SEQ ID NO: 67 - DNA, 21 bp, Homo sapiens
gcgtcacctacctggatgaga

SEQ ID NO: 68 - DNA, 24 bp, Homo sapiens
ccagctgctcgcccgtctacattg

SEQ ID NO: 69 - DNA, 20 bp, Homo sapiens
tggccgctgtgtagaagatg

SEQ ID NO: 70 - DNA, 18 bp, Homo sapiens
cgaatcaccgatcccagc

SEQ ID NO: 71 - DNA, 27 bp, Homo sapiens
cagcaggaaggatcactcggtgaacaa

SEQ ID NO: 72 - DNA, 20 bp, Homo sapiens
cgaagtcacaggaggaggca

SEQ ID NO: 73 - DNA, 25 bp, Homo sapiens
gagaaggtgttggaccaagtctaca

SEQ ID NO: 74 - DNA, 28 bp, Homo sapiens
cctcagtgcatgccctagaccttgagtg

SEQ ID NO: 75 - DNA, 20 bp, Homo sapiens
cttcgtccgatagggtcagg

SEQ ID NO: 76 - DNA, 21 bp, Homo sapiens
actccagaacgggtggaactg

SEQ ID NO: 77 - DNA, 21 bp, Homo sapiens
acccctcccctcttggcagcc

SEQ ID NO: 78 - DNA, 21 bp, Homo sapiens
cgtagggtaaggttcttgccc

SEQ ID NO: 79 - DNA, 19 bp, Homo sapiens
ccggctggtccaggtacat

SEQ ID NO: 80 - DNA, 20 bp, Homo sapiens
ccgagggcctgcagtgctcg

SEQ ID NO: 81 - DNA, 24 bp, Homo sapiens
ttgagcgtgtagtagtcgattcca

SEQ ID NO: 82 - DNA, 24 bp, Homo sapiens
ttagggttagggttagggttaggg

SEQ ID NO: 83 - DNA, 24 bp, Homo sapiens
ttagggttagggttagggttaggg

SEQ ID NO: 84 - DNA, 24 bp, Homo sapiens
ttagggttagggttagggttaggg

SEQ ID NO: 85 - DNA, 24 bp, Homo sapiens
ttagggttagggttagggttaggg

SEQ ID NO: 86 - DNA, 24 bp, Homo sapiens
ttagggttagggttagggttaggg

SEQ ID NO: 87 - DNA, 24 bp, Homo sapiens
ttagggttagggttagggttaggg

SEQ ID NO: 88 - DNA, 24 bp, Homo sapiens
ttagggttagggttagggttaggg

SEQ ID NO: 89 - DNA, 24 bp, Homo sapiens
ttagggttagggttagggttaggg

SEQ ID NO: 90 - DNA, 24 bp, Homo sapiens
ttagggttagggttagggttaggg

SEQ ID NO: 91 - DNA, 24 bp, Homo sapiens
ttagggttagggttagggttaggg

SEQ ID NO: 92 - DNA, 24 bp, Homo sapiens
ttagggttagggttagggttaggg

SEQ ID NO: 93 - DNA, 24 bp, Homo sapiens
ttagggttagggttagggttaggg

SEQ ID NO: 94 - DNA, 24 bp, Homo sapiens
ttagggttagggttagggttaggg

SEQ ID NO: 95 - DNA, 24 bp, Homo sapiens
ttagggttagggttagggttaggg

SEQ ID NO: 96 - DNA, 24 bp, Homo sapiens
ttagggttagggttagggttaggg

SEQ ID NO: 97 - DNA, 24 bp, Homo sapiens
ttagggttagggttagggttaggg

SEQ ID NO: 98 - DNA, 24 bp, Homo sapiens
ttagggttagggttagggttaggg

SEQ ID NO: 99 - DNA, 24 bp, Homo sapiens
ttagggttagggttagggttaggg

SEQ ID NO: 100 - DNA, 24 bp, Homo sapiens
ttagggttagggttagggttaggg

SEQ ID NO: 101 - DNA, 769 bp, Homo sapiens
catcagtatagagaacgttagcctgtggagctgtgaatgtgatggagacaagatttagtgtatagctctgctacctgcctggtgttcctttgagtttctttatccttagatttgacagctgagaaatctaggtggattcatattcgtaatcattgattaacatgcacatttgggtttgcacatttttgtttatcatacatttttctccgttttctattaaagaacatgctctaggggaactattaatagcccaccagtcgggtaggcagcattcaatccttctatgccttctttcgccacctgttgaggtctttcttctgaaacaaagaagaaatagacaaatcagacttgccctcttggaaatgtggtccagatttctctactcccaagctccaaaaaaggcatacattggatgggctagatcaactcctcctgagagccataaatccgccaagagttgttttccatgtaagggtgtggtacaatggggaacgcctgatgttggaggaaagcaggaggactttagagtggagttgcattctaatctctctgccgcttcaactatgtgacctggggcaaatgatataaactctatgagcctctttccttatctttaaaatgaagagaagtaatacctaccttgtagggctgttgtgaggattaaatgaagtaatgcatacagtgcctaacaaagtatttaacatcatattttttaaaagctcatgaaatattagtttttcttccttcccctctttctattttctctcct

SEQ ID NO: 102 - DNA, 1683 bp, Homo sapiens
ggcctccaagcacctcccgcctgcccatcatcgatgtggsccccttggacgttggtgccccagaccaggaattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccaagcacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttggggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcaragacaacttcccaaagcacaaagcagtttttccccctaggggtgggaggaagcaaaagactctgtacctattttgtatgtgtataataatttgagatgtttttaattattttgattgctggaataaagcatgtggaaatgacccaaaaaaaaaaaaaaaaaaaaaaaaaaaaaaaaaaaaaaaaaaaaaaaaaaaaaaaaaaaaaaaaaaaaaccccaaaaaaaaaaaaaggggg

SEQ ID NO: 103 - DNA, 377 bp, Homo sapiens
cgcgtccgggcggctcccgcgctcgcagggccgtgccacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcc

SEQ ID NO: 104 - DNA, 844 bp, Homo sapiens
  misc_feature (108)..(109): any nucleotide
cccacgcgtccgcccacgcgtccgggtcgccctccgtcgtggtctggcgtgtattccgagcsttggtgtctggcggtttccgagcgttggtgtctggcggtttccganngttnnngaccgttggtgtctggcggtttccgaccgttggtgtctggcacgcgccaccctctcttgctttggttgcgccatgccgatgtaccagacaagaagacaagaaaatgatttgaggacagcttcaatcgcggtgtgaagaagaaagcagcaaaacgaccactgaaaacaacgccggtggcaaaatatccaaagaaagggtcccaagcggtacatcgtcatagccggaaacagtcagagccaccagccaatgatmttttcaatgctgcgaaagctgccaaaagtgacatgcagggatgtccttcctgagatccgtgctatctgcattgaggaaattgggtgttggatgcaaagctacagcacgtctttcctcaccgacagctatttaaaatatattggttggactctgcatgataagcaccgagaagtccgcgtgaagtgcgtgaaggctctgaaagggctgtacggtaaccgggacctgaccgcacgcctggagctcttcactggccgcttcaaggactggatggtttccatgatcgtggacagagagtacagtgtggcagtggaggccgtcagattactgatacttatccttaagaacatggaaggggtgctgatggacgtggactgtgagagcgtctaccccattgtgtaggcgtctaattgaggcctggcctctgctgtgggtgaatttctgtactggaaacttttctaccctgagtgcgagataaga

SEQ ID NO: 105 - DNA, 3357 bp, Homo sapiens
  misc_feature (1554)..(1554): any nucleotide
ggccccctgtggtgcccaaccccatacactcttttgtcctsaataccttcctycacwactcactattccgtgcytgatcttaaagatgcttttttcactattcccctgcayccctcrtyccagcctctcyttgctttcacttrgactgaccckgrcacccattaggctcagcaaattaccaaggctgtactgccrcaaggcttcayagacagcccccattacttcagtcaagcccaaatttcatcctcatctgttacctatytcggcataattctcmtaaaaacacacrtgctttccctgctgatcgtgtccgattaatctcccaaacctcaatcccttacaaaacaacaactcctttccttcctaggcatggttmgtgcggtcagaattcttamacaagagccaggactgaaccctgtagcctttctgtccaaacaacttgaccttactgttttagcctagccctcaggtctgcgtacagaggctgccgctgctttaatacttttagaggccctaaaaatcacaaactacgctcaactcactctctacatttctcataacttccaaaatctattttcttcctcatacctgacgcatatactttctgctccccggctccttcagctgtactcactctttcttaagtcccacaattaccgttgttcctggccgggacttcaatctggcctcccacattattcctgataccacacctgacccccacgattgtatctctctgatccacctgatattcaccccatttccccatatttccttctttcctgttcctcaccctgatcacacttgatttattgatggcagttccaccaggcctaatcgccacataccagcaaaggcaggctatgctatagtacaagccactagcccgcctctcagaacctctcatttcctttccatcatggaaatctatcctcaaggaaataacttcccagtgttccatctgctattctactactcctcagggattattcaggccccctcccttccctacacatcaagctcraggatttgcccccacccaggactggcaaaytagctttactcaacatgcctgagtcaggaaactaaaatacctcttagtctaaatagacactttcactgaataagtaaaggcctttcctacagggtctgagaaggcctccgcagtcatttcttccattctgtcagacataattcctcagtttagccttcccacctcaatacagtctgataacagatgagcctttattagtcaaatcagccaagcagtttttcaggctcttagtattcagtgaaacctttatatcccttacrgtcctccrtcttcaagaaargtagaatggactraaggtcttttaaaaacacacctcaccaagctcagccaccaaaaaggactggacaatacttttaycactttcccttctcagaattcaggcctgtcctcggaatgctacarggtacagcccatttaagctcctgtatagaygctcctttttattaggccccagtctcattccagacaccrgaccaacttagactgtgcccccaaaaaancttgtcatccctactatyttctgtctagtcatactcctattywccrttctcaactactcatacatgccctgctcttgtttacactgccggtttacactgtttytccaagccatcacagctgatatctcctggtgctatccccaaacygccactcttaactcttgaagtaaataaataatctttgctggcaggactatgctgaatctccttaggcactctctaatcagatrtcctnggtcntcccaattcttagaccttttatacctgtttttctccttctgttattccatttagtttytcaattcatmcaaaaccgtatccaggccatcaccaatcattctatacracaaatgtttcttctaacawccccacaatatcaccccttaccacaagacctcccttcagcttaatctctcccactctaggttcccacgccgcccctaatcccgcttgaagcagccctgagaaacatcgcccattctctctccataccaccccncaaaaattttcgccgccccaacacttcaacactattttgttttatttttcttattaatataagaaggcaggaatgtcaggcctctgagcccaagccaagccatcgcatcccctgtgacttgcacgtataygcccagatggcctgaagtaactraagaatcacaaaagaagtgaatatgccctgccccaccttaactgatgacattccaccacaaaagaagtgtaaatggccrgtccttgccttaastgatgacattaccttgtgaaagtccttttcctggctcatcctggctcaaaaagcacccccactgagcaccttgcgacccccactcctrcccgccagagaacaaaccccctttgactgtaattttcctttacctacccaaatcctataaaacggccccacccttatctcccttcgctgactctcttttcggactcagcccgcctgcacccaggtgaaataaacagccttgttgctcacacaaagcctgtttggtggtctcttcacacagacgcgcatgaaagggaagacatacaaaaacaaggtaaataagtaaactacgttatatgtttgataatggtgatgttaagggtggggaaagaagaaagcaaagaaggataagaaatgggagggggcaattctagaaaccatagtcagggaagacctcactgagaaggtgacatttgagttatacctgagagatgtgagtatctgagggaaagatattccaggaagggcaaacgttaagtgcaaaggcactgagtgggagtgtgcctggcaggttcaatctattgaaccatgacactggggagggatggtggctactcttggctttgctggctggccactggtgaatgagagacgtaataaagcattcaaattaaagatattaatgcctagtcttcaggcacttagacatctgatgtggagtctgaagttgcagtaacttgagagaagaccatacataactggatagatgcatagatagataaatggatgaatggaattgccttatggccatactgagacacagcaaagccaactcgaatcacgcacggggtaccatggcataggggaaagcactctatgtcatctcagcaacacagctgtgtgcctgggataagtttccttccggagctttcattcttccacagacaagataagaataacatccttaagtggttggtacaccacaggttaaatgttcaatgtttgttatatgccaggctacgtgtattaatacgaatttacttaatccttacaggcctctgaggtaggtactactgagacagccaggtggg

SEQ ID NO: 106 - DNA, 1252 bp, Homo sapiens
tcaatcccctgtcctcctgctctttgctccatgagaaagatccacctacgacctcgggtcctcagaccgaccagcccaagaaacatctcaccaatttcaaatccggtatatgcccagatggcctgaagtaactgaagaatcacaaaagaagtgaatatgctttgtcccaccttaactgatgacattccatcacaaaagaagtgtaaatggccggtccttgccttaactgatgacattaccttgtgaaagtccttttcctggctcatcctggctcaaaaagcacccccactgagcaccttgtgacccccactcctgcccactgagcaccttgcgacccccactcctacccaccagaaaacaaaccccctttgactgtaattttcctttacctwcccaaatcctataaaacggccccacccttatctccgtttgctgactcttttcggactcagcccgcctgcacccaggtgaaataaacagcctcgttgctcacacaaagcctgtttggtggtctcttcacacggacgcgcatgaaatttggtgccgtgactcggatcgggggacctcccttgggagatcaatcccctgtcctcctgctctttgctccgtgagaaagatccacctacgacctcaggtcctcagaccaaccagcccaagaaacatctcaccaatttcaaatccggtaagcggcctctttttactctgttctccaacctccctcactatccctcaacctctttctcctttcaatcttggcgccacacttcaatctctcccttctcttaatttcaattcctttcattttctggtagagacaaaagagacatgttttatccgtgaacccaaaactccggcgccggtcacggactgggaaggcagtcttcccttggtgtttaatcattgcagggacgcctctctgatttcacgtttcagaccacgcagggatgcctgccttggtccttcacccttagcggcaagtcccgctttcctggggcaggggcaagtacccctcaaccccttctccttcacccttagcggcaagtcccgcttttctggggcaggggcaagtacccctcaaccccttctccttcacccttagcagcaagtcccgctttcctagggggcaagaaccccccaatcgcttattttcacgccccaacctcttatctctgtgccccaatcccttatttccacgccccaatctcttatctctgcgccccaatcccttatttctgtgccccaaccccttctc

SEQ ID NO: 107 - DNA, 1501 bp, Homo sapiens
caaagcctgtttggtggtctcttcacatggatgcgcatgaaatttggtgcggtgactcggatcgggggacctcccttgggagatcaatcccctgtcctcctgttctttgctccgtgagaaagagccacctacgacctcaggtcctcagaccaaccaggccaagaaacatctcaccaatttcaaatccggctgctcctcgccaggccgagctagttcccaattcttcctcagcctctcctcctccaccctrtaatctttttatcacctcccctcctcacacctggtccgrcttacagtttcgttcygtgactagccctcccccwcctgcccagcaayttactcttraaaakgtggckggagccaaaggcatagtcaaggttaatgctcctttttctttatcccaaatcrgatagygtttaggctctttttcatcaaatataaaaayccagcccagttcatgrcttgttysgcagcaaccctgagacrctttacagccctagaccctaaaargtcaaaaggccrtcttattctcaaaatacattttattacccaatctkctcccgacattarataaaactccaaaaattaaattccrgccctcaaaccccacaacaggatttaattaacctcgccttcaaggtgtacmataatagaaaaaagttgcaattccttgcctccactgtgagacaaaccccagccacatctccagcacacaagaacttccaaacgcctgaaccgcagckgccaggsgttcctccagaacctcctcccmcakgagcttgctacatgtgccggaaatctggccactgggccaaggaakgcccgcagccygggattcctcctaagccgygtcccatctgtgtgggaccccactgaaaatcggactgttcaactcacctggcagccactcccagagcccctggaactctggcccaaggctctctgactgactccttcccagatcttctcggcttascggytgaagactgacactgcccgatcrcctcggaagccccctagaccatcacgaacgccgagctttgggtaactctcacagtgaaaggcccatccatctggcagagaaagggatgctcaggacacagaacaaccatgctaccttaacaagacttccgtgagcaccaactttggatgcggtctactctctacagaggtctctggcaacctcacaacctgcagttccttgccctcatgcagcacttcctgagaggcagagacgtggactaggagaaacctgagagacacggtctcgctctacacctcaggctggagtgcagtggcacaaacacagctcagtgtaatctagaactcctgggctcaagagatcttcctgccttagcctccggagtagccaggactacaggtatgcaccaccacatccagctgagaatatgcagtcctgctaggatgtaatgaaaatggtactttatcttggtggtattcctccaaaaaacatacaactccaggttaaccatgagagaaa

SEQ ID NO: 108 - DNA, 5507 bp, Homo sapiens
  misc_feature (2144)..(2144): any nucleotide
tttttttttttggaaaataaaaatttatttttaagtcaaagtatgcaacaaataaacctacagaaaacattttcccatcccaatttgttgctttaccaaataatattttgaaaacacattccttcagtcattataaagtttttaaaatacaaaagaaattaaatttgtaagaaagtttagtagaccagatgctgttgtcaagacttgtaaggtggggtttttgctttcagtacatcccacgccatccacctccactcatgccgccttgagaacaaaccccctttgactgtaatttttttttacytacccaaatcctrtaaaacggccccmcccttatytcccttcgctgactytyttttcggactcagcccrcctgcacccaggtgaaataaacagccwtgttgctcacacaaagcctgtttggtggtctcttcacasggacgcgcatgaaatttggtgycgtgactcggatcgggggacctcccttrggagatcaatcccctgtcctcctgctctttgctccgtgagaaagatccacctacgacctcaggtcctcagaccgaccagcccaagaaacatctcaccaatttcaaatccggtaagcggcctctttttactctcttctccarcttccctcactatccctcaacctctttctcctttcaatcttggygccacacttcaatctctcccttctcttaatttcaattcctttcattttctggtagagacaaaggagacacrttttatccgtggacccaaaactcyggcgycggtcacggactgggaaggcagccttcccttggtgtttaatcattgcaggggcrcctctctgattattcacccacgtttcaaaggtgtcagaccacgcagggaygcytgccttggtccttcacccttagcggcaagtcccgcttttctggggaaggggcaagtaccccaaccccttctctccttgtctctaccccttctctgcttttctgggggagggacaagtacccctcaaccccttctccttcacccttaatggcaagtcccgcttttctgggggaggggcaagtacccctcaaccccttctccttcacccttagtggcaagtccygykttyctagggggcaagaacccccaatcccttatttccgcaccccaacctcttatctctgtgccctaattccttatttccatgccccaaccctttctctgcttttctggagggcaaraaacccctaccgcttctccgtgtctctactcttttctctgggcttgcctccttcactatgggcaagtttccaccttccattcctccttcttctcccttagcctrtattcttaagaacttaaaacctcttcaaytctcacctgacctaaaatctaagcrtcttattttcttctgcaatgccgcttgaccccaatacaaactcgacagtagttccaaatagcyrgaaaayggcactttcaatttttccatcctrcaagatctaaataattcttgtwgtaaaatgggcaaatggtctgaggtgcctgacrtccaggcattcttttacacatcagtcccytcctagtctctgtgcccagtgcaactcstcccaaatcttcyttctttccctccckcctgtcccctcagtaccaaccccaagtgtcgctgagtctttctaatcttccttttctacagacccatctgacctctcccctcctcgacaggctgagctaggtcccaattcttcctcagcctccactcctccaccctataatctttttatcgcctcccctcctcacaccygktcyrgcttacagtttcrttccgtgacyagccctcccccacctgcccagcaatttaytcttaaaaaggtggctggagccaaagtcataatcaaggtgaatgctcctttttctttatcccaaatcagatagcgtttaggctctttttcatcaaatataaaaatccagcccagttcatgacttgtttggcagcaaccctgagacgctttacagccctggaccctaaaaggtcaaaaggctgtcttattctcaatatacgttttattacccaatctgctyccgayattaaataaaactccaaaaattrgaatctggccctcaaaccccacaacaggatttaattaacctcrccttcaaggtgtacnataayagaaaaaagttgcaattccttgcctccwctgtgagacaaaccccagccacatctccarcacacaagaacttccaaacgcctraaccgcagcrgccaggcgttcctccagaacctcctcccmcaggagcttgctacaygtgccggaaatctggccacygggccaaggaatgcccgcagscygggattcctcctaagcygygtcccatctgtgtgggaccccactgaaaatcggactgttcaactcacctggcagccaytcccagagcccctggaactctggcccargsctctctgactgactccttcccagatcttctcggcttagcggctgaagacygacactgccsgatcacctcggaagccccstagaccatyatggacgccragctttrggtaactctcacagtggaaggtargcccrtccccttcttaatcaatayggaggctacccactccacattaccttcttttcaagggcctgtttcccttgcctccataactgttgtgggtattgacagcyaggcttctaaacytcttaaaactccccaactctggtgccaacttagacaatactcttttaagcactcctttttagttayccccacctgcccagttcccttattaggctgagacactttaactaaattatctgcttccctgactattcctgggctacagccacacctcattgctgccttttcccccartycaaagcctccttcrcatcctccccttgtatcyccccaccttaacccacaagtataagatacctctactccctccttrgcgaccgaccatgcrccccttaccatctcattraaacctaatcaccyttaccycactcaacgccaatatcccatcccgcagcacgctttaaaaagattaaagcctgttatcactcgcctgctacagcatggccttttaaagcctataaactctccttacaattcccccattttacctgtcctaaaaccagacaagccttacaagttagttcaggatctgcrccttatcaaccaaattgttttgcctatccaccccgtggtgccaaacccatatactctcctatcctcaatacctgcctcyacaacccattattctgttctagatctcaaacatgctttctttactattcctttgcacccttaatcccagcctctcttcgctttcacttggactgaccctgacacccatcaagctcagcaaattacctaggctgtactgcygcaaagcttcacagacagcccccattacttcaatcaagcccaaatttcttcctcatctgttacctatctcggcataattctcataaaaacacacgtgctctccctgccaatcgtgtcygactgatctctcaaacccmagcaccttctacaaaacaacaactcctttccttcctaggcatggttagcntggtcagaattcttacacaagagccaggaccacaccctgtagcctttctgtccaaacaacttgaccttactgttttagcctagccctcatgtctgcgtgcagcrgctgccrctgctttaatacttttagaggccctcaaaatcacaaactatgctcaactcactctctacagttctcataacttccaaaatctattttcttcctcatacctgacrcatatactttctgcttcccggctccttcagctrtactcactctttgttgagtctcccacaattaccattgttcctggcccrgacttcaatccggcctcccacattattcctgataccacacctgacccccatgactgtatctctctgatccacctgacattcaccccatttccccaaatttccttctttcctgttcctcaccctgatcacrcttgatttattgatggcggttccaccaggcctaatcgccacacaccagcaaaggcaggttatgctatagtacaagccactagcccgcctcttagaacctctcatttcctttccatcgtggaaatctatcctcaaggaaataacttctcagtgttccatctgctattctactactcctcagggattattcaggccccctcccttccctacacatcaagctcraggatttgccccacccaggactggcaaattagctttactcaacatgccctgagtcmsataactaaaatacctcttagtctaggtagatactttcactggatagrtasaggcctttcctacagggtytgagaaggccaccrcagtcatttcttccrttctgtcagacataattcctcagtttagccttcccacctcaatacagtctgataacagacsagcctttattagtcaaatcagccaagcagtttttcaggctcttagtattcagtgaaacctttatatcccttatggtcctccgtcttcaagaaaagtagaatggactaaaggtcttttaaaaacacacctcaccaagctcagccaccaacttaaaaaggactggacaatacttttaccactttcccttctcagaattcaggcctgtcctcrgaatgctacagggtacagcccatttaagctcctgtatagacgctcctttttattaggccccagtctcattccagacaccagaccaacttagactgtgccccmaaaaaacttgtcatccctactatcttctgtctagtcatactcctattcaccgttctcaactactcatacatgccctgctcttgtttacactgcyggtttacactgtttttccaagccatcacagctgatatctcctggtgctatccccaaactgccactcttaactcttgaagtaaataaayaatctttgctggcaggactatgctgaatctccttargcactctctaatyagatrtcctrrgtcntcccaattcttagaccttttatacctgtttttctccttctgttattccatttagtttytcaattcatccaaaaccrtatccaggccatcaccaatcattctataygacaaatgtttcttctaacatccccacaatatcaccccttaccacaagacctcccttcagcttaatctctcccactctaggttcccacrccgcccctaatcccgcttgaagcagccctgagaaacatcgcccattctctctccataccaccccccaaaaattttcrccgccccaacacttcaacactattttgttttrtttttcttattaatataagaaggcrggaatgtcaggcctctgagcccaagccaagccatcgcatcccctgtgacttgcayrtatacryccagatggcctgaagtaactgaagaatcacaaaagaagtgaatatgccctgccccaccttaactgatgacattccaccacaaaatggccggtatttatttattccactggtaaatggccgggccttgccttaa

SEQ ID NO: 109 - DNA, 1997 bp, Homo sapiens
  misc_feature (1063)..(1063): any nucleotide
gacccacgcgtccgcccacgcgtccgccccactcaatgccaatatcccatcccgcagcacactttaaaaagattaaagcctgttatcactcgcctgctacagcatagtcttttaaagcctataaactctccttacaattcccccattttacctgtcctaaaaccagacaagccttacaagttagttcaggacctgcacattatcaatcaaattgttttgcctatcgaccctgtggtgcccaacccatacactcttttgtcctcaataccttcctccacaactcactattccctgcttgatcttaaagatgcttttttcactattcccctgcacccctcgtcccagcctctctttgctttcatttggactgaccctgacaccatcaagctcagcaaactacctaggctgtactgccgcaaagcttcacagacagcccccattacttcaatcaagcccaaatttcttcctcatctgttacctatctyggcataattctcataaaaacacacgtgctctccctgccaatcgtgtccgactgatctctcaaacccmarcaccttctacaaaacaacaactcctttccttcctrggcatggttagcacagtcagaattcttacacaagarccaggaccacaccctgtagcctttctgtccaaacaacttgaccttactgttttagccyagccctcatgtctgygtgcagcggctgccrctgctttaatacttttaraggccctcaaaatcacaaactrtgctcaactcactctctacagttctcataacttccaaaatctattttcttcctcatacctgacgcatatactttctgcttcccggctccttcagctgtactcactctttgttragttcccacaattactgttgttcctgrcccagacttcaatccggcctcccacattattcctgataccacacctgacccccatgactgtatctctstgatccacctgacattcaccccatttccccaaatttccttctttcctgttcctcacyctgatcacgcttgatttattgatggtggttccaccaggcctaatngccacacaccagcaaaggcaggttatnctatagtacaagccactagccyrcctcttagaacctctcatttcctttccatcgtggaaatctatcctcaaggaaataacttctcagtgttccatctgctattctactactcctcagggattattcaggccccctyccttccctacacatcaagctcgaggatttgccccacccaggactggcaaattagctttactcaacatgccctgagtcagataactaaaatacytcttagtctaggtagatactttcactrgataggtagaggcctttcctacagggtctgagaargccaccacagtcatttcttcccttctgttagacataattcctcagtttagccttcmgcacctcaatasagtctgataacagatgagcctttattagtcaaatcagscaagcagtttttcaggctcttagtattcagtgaaacctttatatcccttacggkcctccrtcttcaagaaaagtagaatggactaaaggtcttttaaaaacacacctyaccaagctcagycaccaacttaaaaaggactggacaatacttttaccactttcccttctcagaattcaggcctgtcctyggaatgctacagggtacagcccatttaagctgctgtatagacataacttggcccatgatagctagtattcagttcttccttttatgcacaaccacagccagcaggaagctaccagagaatatgcaccagtgaaataaggtgtgtaaataaaaaagatatgcaatccatgaaacagaacatccagccaaggatcataacagcaaatgccagctctggtgagcacgttatattgaaaagggtgtgactgtggtgaaagacttgccacaaatcatgaaacaaaaccaaccagcactgacagatcatttaaaatgtttaaatacttg

SEQ ID NO: 110 - DNA, 1920 bp, Homo sapiens
ccgcctgcacccaggtgaaataacagccatgttgcttacacacagcctgtttggtggtctcttcacatggacgcgcatgaaatttggtgccgtgactcggatcgggggacctcccttgctagatcaatcccccgtcctcctgctctttgctccgtgagaaagatccacccacgacctcaggtcctcagaccaaccagcccaaggaacatctcaccaattttaaatcagatcttctcggcttagcggctgaagactgrcactgccssatcrcctyggaagccccctagaccrtcacwgacgccgagcttcaggtaactctcacagtggaaggtaagcccgtcyccttcttaatcaatacrgaggstacccactccacrttaccttcttttcaagggcctgtttcccttgcctccataactgttgtgggtattgacrgccaggcttctaaacctcttaaaactccccaactctggtgccaacttagacaatactcttttaagcactcctttktagttatcccyacctgcccagttcccttattaggctgagacactttaactaaattatctgcttccctgactattcctggactacagctatatctcattgccgcccttcttcccaatccaaagcctcctttgcgtcctcctcttgtatccccccaccttaacccacaagtataagatacstctactccctccttggygaccgatcatgcaccccttaccatctcattaaaacctaatcacccttacccyactcaacgccaatatcccatcccgcagcacrctttaaaaagattaaagcctgttatcactckyctgctacagcatggccttttaaagcctataaactcyccttacaattcycccattttacctgtcctaaaaccrgacaagccttacaagttagttcmggatctgtgccttatcaaccaaattgttttgcctatccacccygtggtgccaaacccrtatmctctcctatcctcaatacctscctctacwacccattaktctgttctagawctcaaacatgctttctttactattcctttgcacccttcatcccagcctctctyyrctttcacttrgactsaccctgacacysatyargctcagcaaattacctrggctgtactgccrcaargcttcacagacagcccccattacttcartcaagcccaaatttcwtcctcatctgttacctatctcggcataattctcataaaaacacacgtgctytccctgcyratcgtgtccgaytratctcycaaacccaakccctttacaaaacaacaactcctttccttcctaggcatggttagcgcggtcagaattcttacacaagagccaggaccacaccctgtagcctttctgtccaaacaacttgaccttactgktttagcctagccctcatgtctgcgtgcagmggctgccgctgctttaatacttatagaggccctcaaaataagtagaggcctttcctacagggtctgagaaggccaccgcagtcatttcttcccttctgtcagacataattcctcagtctagccttcccacctcaatacagtctgataacagacgagcctttattagtcaaatcagccaagcagtttttcaggctcttagtattcagtgaaacctttatatcccttatagtcctccatcttcaagaaaacacmcctcaccaagctcagccaccaacttaaaaaggactggacaatacttttaccactttcccttctcagaattcaggcctgtcctcagaatgctacagggtacagcccatttaaggtcctgtatagatgctcctttttattaggccccagtctcattccagacaccagaccaacttagactgtgcctcaaaaaaaaaaaaaaaaaaaaaaaaactcgagactagttctctctctctccc

SEQ ID NO: 111 - DNA, 1943 bp, Homo sapiens
gggagagagagagagagagagagagagagagagagagagagagagagagagagagagagagagagagagagagagagagagagagagagagagagagagagagagagagagagagagagagagagagagagagcgtgtctctactcttttctctgggcttgcctccttcactatgggyaagyttccaccttccattcctttcttctcccttagcmtgtrttctyaaraaytwaaaayctcttcaactcwcacctgacctaaaayctaarygycttattttcttctgcaatgccrcttgaccccaatacaaactcracagtagttccaaatagccagaaaatggcactttsaatttttccamcctrcaaratctaaataattcttgkcrtaaaatrggcaaatggtgtgaggtgcctgacgtccaggcattcttttacacatcagtcccttcctagtcyctgtgcccagtgcaactcgtcccaaatcttccttctttccctcccgcctgtcccctcagtaccaaccccaagcgtcactgagtctttctaatcttccttttctacagacccatctgacctctcccttcctccccaggctgctccttgccaggccgagctaggtcccaattcttcctcagcctctgctcctccaccctataatctttttatcacctcccctcctcacacctgctccggcttacagtttcattccgtgactagccctccccgacctgcccagcaatttattcttaaaaaggtggctggagctaaacgcatagtcaaggttaatgctcctttttctttatcccaaatcagatagtgtttaggctctttttcatcaaatataaaaatctagcccagttcatggctcgtttggcagcaaccctaagacactttacagccctagcccctaaaaggtcaaaaggccatcttattctcaatatacattttattacccaatctgctcccgacattaaataaaactccaaaaactggaatctggccctcaaaccccacaacaggacttaattaacctcaccttcaaggtgtgaaataacagaaaaaagttgcaaytccttgcctccactgtgagacaaaccccagccacatctccagcacacaagaacttccaaacgcctgaactgtagcagccagacgtttctccagaacctcctcccccaggaacttgctacacatgccggaaatctggccactgggccaaggaacgcccgcagcccgggattcctcctaagccgcgtcccatctgtgtgggaccccactgaaaatcggactgttcaactcacctggcagccactcccagagctcctggaactctggcccaaggttctctgactgactccttcttggcttactggctgaagactgacgctgcctgatcgcctcagaagccccgcagaccatcatggacgccgagctttagcccgcctgcacccaggtgaaataaacagccttgttgctcacacaaagcctgtttggtggtctcttcacacagacgcgcatgaaagggaagacatacaaaaacaaggcctctgaggtaggtactactgagacagccaggtgggaaggactccttggcaaaactccaaccagcctgtacactgggaggaatgtgcactgggatggagccatagaagtttgtgtcgtttgcagtggggaggagcctggtccctcctcttcctgtgaggaacctggaattcaatctgtgaggaacttcttgaaagacccatcaattcttcaatagaaagcatcaaaggacaatttacaccctaagactgaacccctgacctcaaaatctttcccttgctatgttcaccaacctcaacagaaatattaggattcttacctgatcctagccaagccccctccctcatctcccattaaagggtccatcttcaaccaaacttaagtctcaataaatatctg

SEQ ID NO: 112 - DNA, 2286 bp, Homo sapiens
gggtgagccccgtgcccggcccaatttttgtatttttagtagagacgggttcaccatgttggccaggctagtcttgaactcctgacctcaggtgatctgcctacctcagcctcccgagtagctgggattacaggtgcctgccaccacgcctggctaattttttgtatttttagtagagaaggggtttcaccatattagccacaatggtctcaatctcctgacctcgtgatccatctgccccgccctcccagagtgctgggattacaggcgtcagccaccgtgaccggctcagactgtactcttatagccatctgaaatacgttttctaggtagagatagattgtgtaagggtacagttgtgaggataacagaaacatggcagattatttaaaatcatcctgaaagtggtgctttatctgatgaaagtgattgtaatccataggaaaatgtttcaacgtgcgcaagagttgcggcggcgggcagaggactaccacaaatgcaaaatccccccttctgcaagaaaggctctttgcaactgggtaagtttgcttgttttccttgcttttggacatagtctgccaggtcaggacatggatacatttttctccctacagctctgtgctcaagccctgcagagggagatggcagagagaaaggctgcctacaagcatcacagtcccatccctgtkggkaaccgtgttgygcaaaaacaccttcatccccacccagtggggcccctgatctaatattctaagtgtcagaggttccgtatttgtaatagcaratgggccctgactgtaaaytagtgaagagtgaatgtaacttattacccacagggacaattccaaatgarggccttaaatgatgctcagctaagctggttcttgtgtggcctctgtaccttcaaaagctgccgagtcctatgattgcacgcgatgggacttgtacacttgaagtgaaacacagttttaaaacttgctttgtttagaattcccacctcatttttccatggacaaaagtattctttatgtcctagtgcacttacaatttggtattacctgggagtgaaaagaaatattacagccatgcctaastgacttcttgaggtaagattgttctgtcagaaaaccctctcccagttcccctgcagctcttcaggaatccacatctctccagagctctttgttctcatgggtggcacctccagagtgaagaagatcctttgtcaagaagggaaacagaggggaaatgagagggtcctgcaggcagagctggaatcaacttccactctgcctcttgcaagctgtgtgaccctgggcacaatttctccttcctctggaaacctctgttttcttagatttggagcaggrtggtcacactgaccttgcagagttctgagaatcagagacagaacataaaaggcctggaaaacattctccaaaaagaagctgcaacatgtgtggacaatgggcttttcatgcctctcttactgtctcttactgkctattgacctggtgcaagaaacatgctctggtgatggctgtgagggaggaatgaggatagacatagacactcctgtgtctcaaacatgcttctttattactctgttatgactctgtcttccctggggcaggaccccagcctgcctacatttgcagacagacacagtggcatgtggagacaacagtgtgtcccartgacttttctttacccccyagctgtcggcagtactcagtggaagggtgatatgacactgayactgctattttgaaacctggaggatggaaaggtgcaaaaatctatcaccagcaacagaaggtgcagactgtgttggtggcggtaattttgtccatcaaatgaatatgtgtgaaaacattccctcctttggccctacaggtcagaatggcggcagyrgagcatcgtcattcttcaggattgccctrctggccctacctcacagctgaaactttaaaaaacaggatgggccaccagccacctcctccaactcaacaacattctataattgataactccctgagcctcaagacaccttccgagtgtgtgctctatccccttccaccctcagcggatgataatctcaagacacctcccgagtgtctgctcactccccttccaccctcagctctaccctcagcggatgataatctcaagacacctgccgagtgcctgctctatccccttccaccctcagcggatgataatctcaagacacctcccgagtgtctgctcactccccttccaccctcagctccaccctcagcggat

SEQ ID NO: 113 - DNA, 1280 bp, Homo sapiens
cagcattcagattgccttttctctcaaccaggatctttaaagtcgatgacaagagttccagtcctgaatcatggcaaagtgcagtagtgaactgcggggttattctggaaggatctctctatggctgatggtctcagttccggcatcagcctctgactgagaatcaggtctcacacaggaggagtcagatgaggagcaatcctctgcttccgatggagttagttgtgatgaattggtgaggtctggtttttcacactgaactaaaatgagctttcgctgtgtcaagcacaagactgaccccagagacacacatagtgcacctcatagaagcttttaatagtctttatatttactaaagaataggactaactatggaactatgaagatgagctggaaatgacaggtgacttgccagcaggccagagtgtgayttttttttgtccctcaatgggaggtgtcyattctcccttygsttgtgagaatcagttggttcatttgtgggaaggttgcaggggggatctttgaatcacagccttcagatgccagaagggcagagggaatcccacacgggctggtggatcatgtgtgtgcatttctctcccttctartctgaggaaactaagcrtgaaagaaygtgagcaygsagaaaaggagaggcaggtrtcagaggcagaggaaaaygggaaattggatatgaaagaaatacacacctacaagtgagttcagaaactgaaccccaccctcytgggaaacgcccattggagtgttgtttttaacctytgtacaatgtttagacccagtaaatgcagaaatagaaacaaatggtcagaagacatatcgtgagagagagagagagagttcacaaaacagaaaacaaagtaccttaatatttaccagtgaccaaaagatgtgaagcagcaaaaggtctcctgaccccattgccagctagactgtgtagaaactcggttcataccagccattctaggggtggggtgagtttgttgtcatccttaggaaagtgtgttgttgtaggatcaaccacatccttcaaaaggactatgcctgtttataagcccagctgtttctgccctgtgaaacacggtaaggatattaatacaaagagaatacagctttatgataaaagatgctcagtgaaggatgaattagggatatactgagaatggggaaggaaactatcatctcagaagtcagcaggcagtaagcaagaggaggaatcaatatagcaacagtttggatcagactgtacagtttttttttgt

SEQ ID NO: 114 - DNA, 2247 bp, Homo sapiens
ggcgtgaggcgccgcccgggtgtccccgcggcgcaggaggcggtggagcgcagagcgggcgagcgcgaaaaatcactaccaatataatggattttatatatcagattgctttattctggatatcatggtaacaatacagaaagctcctacgtgtacctggagggccgctgcctcaattgcagcagcggctccaagcgagggcggtgggctgcacgtacgttcagcaacaagacactggtgctggatgagaccaccacatccacgggcagcgcaggcatgtgactggtgctgcggcggggcgtgctgcgggacggcgagggatacaccttcacgctgacggtgctgggccgctctggcgaggaggagggctgcgcctccatccccctgtcccccaaccgcccgccgctggggggctcttgccgcctcttcccactgggcgctgtgcacgcyctcaccaccaaggtgcacttcgaatgcaygggctggcatgacgcggaggatgctggcgccccgctggtgtacgccctgctgctgcagcgctgtcgccagggccactgcgaggagttctgtgtctacaagggcagcctctccggctacggagccgtgctgcccccgggtttcaggccacacttcgaggtgggcctggccgtggtggtgcaggaccagctgggagccgctgtggtcgccctcaacaggtctctggccatcaccctcccagagcccaacggcagcgcaatggggctcacagtctggctgcacgggctcaccgctagtgtgctcccggggctgctgcggcaggccgatccccagcacgtcatcgagtactcgctggccctggtcactgtgctgaacgagtacgagcgggccctggacgtggcggcagagcccaagcacgagcggcagcgccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgcgcatcctgcaggcagagaccaccgcgggcaccgtgacgcccaccgccatcggagacagcatcctcaacatcacaggagacctcatccacctggccagctcagacgtgcgggcaccacagcgctcagagctgggagccgagtcaccatcgcggatggtggcgtcccaggcctacaacctgacctctgccctcacgcccatcstcacgcgctcccgcgtgctcaacgaggagcccctgacgctggcgggctttsagsagggccccggscaacctcrgtgaygtggtgcagctcatctttctggtggactccaatccctttccctttggctatatcagcaactacaccgtctccaccaaggtggcctcgatggcgttccagacacaggccggcgcccagatccccatcgagcggctggcctcagagcgcgcctcaccgtgaaggtgcccaacaactcggactgggctgcccggggccaccgcagctccgccaactccgttgtggtccagccccaggcctccgtcggtgctgtggtcaccctggacagcagcaaccctgcggccgtgctgcatctgcagctcaactatacgctgctggacggtgcatgcagcggttggggcacacgcggccccctggccttgttcttggggggaaggcgtttctcgtagggcttccatgggtgtctctggtgaaatttgctttctgtttcatgggctgctgggggcctggccggagaggagctgggggccacggagaarcaggccgctacctgtctgaggaacccgagccctacctggcagtctacctgcactcggagccccggcccaatgagcgcaactgctcggctagcaggaggatccgcccagagtccctccagggtgccgaccaccggccctacaccttcttcatttccccggggaccagagacccagtggggagttaccgtctgaacctctccagccacttccgctggtcggcgctggaggtgtccgtgggcttgtacacgtccctgtgccagtacttcagcgaggaggacgtggtgtggcggacagaggggctgctgcccctggaggagacctcgccccgccaggccgtctgcctcacccgcacctcaccggcttcggcaccagcctcttcatgcccccaagccatgtacgcttttgtgtttcctg

SEQ ID NO: 115 - DNA, 684 bp, Homo sapiens
ggccggcaggcagcgatggcggccgtacggggcctgcgggtgtcggtgaaggcggaggccccggcggggccggccctggggctcccgtcccctgaggcggagtccggtgttgaccgtggcgagccggagcccatggaggtggaggagggcgagctggaaatcgtgcctgtgcggcgctcgctcaaggaactgatcccggacacgagcagaagatatgaaaacaaggctggcagcttcatcactggaattgatgtcacctccaaggaagcaattgaaaagaaagagcagcgagccaagcgcttccattttcgatcggaagtaaatcttgcccaaagaaatgtagccttggaccgagacatgatgaagaaagcaatccccaaggtgagactggagacaatctatatttgcggagtagatgagatgagcacccaagatgtcttttcctattttaaagaatatcctccagctcacatcgaatggttggatgatacctcctgtaatgtagtttggctggatgaaatgacagccacacgagcacttatcaatatgagctccctgcctgcacaggataagatcagaagcagggatgccagtgaggacaagtcagctgagaaaaggaaaaaagacaagcaggaagacagttcagatgatgatgaagctgaagaaggagaggttgaagatgag

SEQ ID NO: 116 - DNA, 613 bp, Homo sapiens
ggcggtgccacccctccccccggcggccccgcgcgcagctcccggctccctcccccttcggatgtggcttgagctgtaggcgcggagggccggagacgctgcagacccgcgacccggagcagctcggaggcggtgaagtcggtggctttccttctctctagctctcgctcgctggtggtgcttcagatgccacacgcgtcccgggggcccggttctccgctcccctcccctccccttctcgccggaccccgcgccgggagctgcgggaaggagtggagggtcgggcggtggcctcgcggctggcctggcgcgcggccagcgccggtagttagtggggggactgctctgccctcgagggggtagggagctgtggcgacggttgccccatttcgagacaaagcgcatttccccctcccctcccccacccgcgttccggcggaggcgccccctcccccagccgccacgcggggctgggtcgagacttgggcctcccggagggcggcgcgtggtcccgcgtccgcgaggcctggcggcgcgcggccggctgtcccgaggctgcggcgaccgcccagttaacgtggccgccgcgggggtaggcgcgtgcggtgtggcg

SEQ ID NO: 117 - DNA, 1006 bp, Homo sapiens
caagcaatagcgcaaaatttaggagacaggatccttgcaaatttaaaaggtgaatgtagtgagggggatggcaagtggctggtacaggctgtggtgattccttttactcaagggtttttgtggagtatagggagaaggggttgatatttatggacacctatgtgtcaggcactgtgcatcattttatccttacaggatgttgtgaggtaggtattattgttttcatttttacaggtgaagaaagcaggtctcagagggactaaaatcctgcccaaggttagtggtagagctgggatccaaaaatctgtcagaatcctgagactgcgctgttccactgtgccacgcagacagttcattcagtttagatgtcacatagtcaagagggaactctatgcatcctttaattttttagactatgatattctttttaaaaattagcctttattttctaactaccaaaagaaatatgaaagcattacagaaacactggaaaatagaaaagaaaaaataaaatcacttacaaccactttttgttttttggagtctcgctttgccacccaggctggagtgcagtggtgtgatcatggctcattgtagcctcaacctcccaggctcaggtaatcctcctgtctcagcctcctgaatagctggaaccacacacacacacgcacacacaggtgtgtgccaccacacccagctatttttttgtattttcttttgtaaagacaaggtttcaccatgttgcccaggctggtctcagagtcctgagctcaaacgatctgcctgccttggcctcccaaaatgttgggattacaggcatgagccaccacatctgacctacaaccactttttaatgtgwgacttaaaaatcttagataaataaggctgtgaagcaaaaccagggatttttttgtttgtttttgatttgcaaaacaagtgactgacaattattgagaaattaaagatagctatgtgtaggtcttgcccctgcgggtttggaggtttc

SEQ ID NO: 118 - DNA, 1916 bp, Homo sapiens
cccacgcgtccgcacgaaagaagtgccttttgcctcccgtcatgattctgaggcctccccagccatgtggaactgtttgaggcacagagctgtatatacaataacagtgaaattgatcccactactaattatgacaaaaatgatcttccacgtaaacaggtggtgaagctccttatggtcctgaccctacagttcctgtcccatgaccagggccagatcaccaaggagctgcagcagttcgtcgtcagtggcagccccatgcgagcacccgaggaaggcaagtacgtgggtgatatattcctgtattcttggacaagtacactggtgacatgtagctgtattcagagtcacaggtgcccaggccggagtgcagtggcgtgatctcggctcgctacaaccaccacctcctagcagcctgccttggccttccaaagtgctgagattgcagcctctgcccrgccgccaccccgtctgggaagtgaggagcgtctctgcctggccgcccatcgtctgggatgtgaggagcccctccgcccagcagccgccccgtctgagaagtgaggagcccctcagcccggcagccaccccatctgagaagtgaggagcccctccacctggcagccaccccgtctgggagggctgkgaccgtctatgacaagccagcatctttctttcaagagacctctggacctgcagcaccaactcttcatgaagctgggcggcacgcactctccgttcagggcctgaacctgaggacccagacacggagcggtcggccttcatggagcgggatgctgggagcgggctggtgatgcgcctccgcgagcggccagccctgctggtcagcagcacaggctggacagaggacgaagacttctccatctgctggcagctttagaaagagtttgaacaactgactcttgatggacacaaccttccttctctcgtctgtgtgataacaggcaaagggcctccgagggagtattacagccgcctcatccaccagaagcatttccagcacatccaggtctgcaccccttggctggaggccgaggactacccccgcttctagggtcggtggatctgggtgtctgtctgcacacgtcctgcagtggcctggacctgcccatgaaggtggtggacatgttcgggtgctgtttgcctgtgtgtgccgtgaacttcaagtggcaggagcagaacccgaatctttctggggatagcttcacagatccaccgctgaggaggaaacagtgcagagcgagctgcccacagtgaggccctgctcctggtttacatgagctggkgaaacatgaagaaaatggcctggtctttgaggactcagaggaactggcagctcagctgcaggtgcttttctcaaactttcctgatcctgcgggcaagctaaaccagttccggaagaacctgcgggagtcgcagcagctccgatgggattagagctgggtgcagactgtgctccctttggttatggacacataactcctgggccagaggctaaaaccccgggacccctgctgtccttcccacagcttcttctcagagtctcagggcaaatcctttcgagcagcgcctcccagtggccagaagctgaaatgatggcagtagtgccacctggtgaatgaattggttctgtgacccgggaagctgtgcttggctctgatttcttttctggaggctcggaaacacttcctctcttcttctgttcttcacgccccatgcccctgctagcgtattactgttctgtgacttccctgtgacctctgcagtactcctcatcctgcgtttggtctccaggtgtcacctttctgccgtgttcctaacattttgattcctgtcttgaaaaaagcacctgctgcaccataagcccagggatgtggcagctgcagcgggcttggctt

SEQ ID NO: 119 - DNA, 1168 bp, Homo sapiens
ctgccatcctctgggcctgaggctgcctggcccagcccctcctaactccctggactcttccacggtgtcttcaggcccctacaccatcctttgtgtaaggggaggtggcagcatagagatgatgggggaactgccccatgtgccaaggaaagctcacccatctgtgcgaaatgctctggttgacattgggtttttgcgcaccaaactgggccatgaccaaggtttataaccaaggtgtctccgggcatgggcactttggctcttgtagaaaccaccccactggcaggagacggcggtagctgtggtcattgaaaacaagctcctgctgataaatctcagacaccagacacagaagaacctggagaccctgccagagagcttgaggcaaatggatggactgttggagcagctgagggtgaagcagcacaaactcctcaaagttgaatagcaaagcagccaccagagatggacaagaaaaatgaacaaagaaaattagcagaaatcaaaggcagatgctaaagcagtgcaaaatcattcattcaatgatagaaatgaaattgatgaaggagtctggaaaatgaatgacagaagagaattaaacagcagtgaccatagtaaggtcctgacgattctggtccactgaatcccatcatccctaagacagtaaatatcatcacagtcaccaccmgcaagttaccaccacagcatttcctgtttgttccaaaatgaataaagatgattctcatcacaagggcaaatacaaagtagtttagtatgtttttaactaaacttcaggtgtttggtttactttttctaagttctcataattctgaaaatgcagttgacacttgtgtggctcatgatgtttttaatagtctaatgctacttgaattgttcaaaaaccactgtattttaaattaagatgaataaacggtcctttgaaaactggcacaaggcaaggatgccctctgtcaccactcctattcaacacagtattggaagttctggccagggcaatcaggcaagggaaagcaatacagcgtatcaaaataggaagagaggaagtcaaattgtctctgtttgcagatgacatgattgcatatttagaaaaccccatcttctcagcccaaaacctccttaagctgataagccaccttcagcagtctca

SEQ ID NO: 120 - DNA, 475 bp, Homo sapiens
ctgtggggaagcggggccgctggtccggaggtagcggtgccggccgagggggtcggggcggctggggcggtcggggccggcgtcctcgggcccagcggtctccatcccggggcacgctggacgtagtgtctgtggacttggtcaccgacagcgatgaggaaattctggaggtcgccaccgctcgcggtgccgcggacgaggttgaggtggagcccccggagcccccggggccggtcgcgtcccgggataacagcaacagtgacagcgaaggggaggacaggcggcccgcaggacccccgcgggagccggtcaggcggcggcggcggctggtgctggatccgggggaggcgccgctggttccggtgtactcggggaaggttaaaagcagccttcgccttatcccagatgatctatccctcctgaaactctaccctccaggggatgaggaagaggcagagctggcagattcgagtgg

SEQ ID NO: 121 - DNA, 1770 bp, Homo sapiens
gggttcttccttttctcttagcgactcctgtgtgtgtctgctgaggtgccctgtccgctggtgctgtgctctgacttactaacccagcccctactaaccctgttttctcttcttactaaccccagccctgccgagctctgggctccccccgggggctggtccccctccttttggcaagcagatgacctggggctactggccctgtagacagatgtcccactttgctgccccatattggctgtaagatcagagtccactgggccaggtctaaggcaggggatggccctattaacaagactcagaggaggaagaggtggtcctgtggatgtgggaggctggactctgagtatgacatctctcctatgtgcagaagtctggttgccactgggagtaggtgggaccagggaaatctctgggacgtgagtgtggaggcctgttggtctagactctagactgtggagctctgagcttttgtgtcctctggaaggaagctggggaagaatcctctccattgttaagtgacagggatagaagctgtcctgcacaggaagtcacgaggggggcgtatcccacgaggaaggcaggagggggcgtgcccctcaccggaaattagcagaggggcgtgtcccacacaggaagtcagaaagcggagcctttcttacaccggaagtcaatgaagcgggtctttcctacgctaaaaaccactgagtggagtatttagtacacaggaagtcggccagagaaacatttctcatatttgaaggccggaaagagggacatttctgacaccggaagtcagtgagaggactctttcccacacaggaagtcagctagagagccgtctcccctctctggagccgagagaggccggtttcccccaccgkaagtagacgtggggccgtgaccggaagtccttgggaaagatccgtyccattcccggaagctagagggcgttagttgtcgggttgaaaaggggtgtggggaggggaagcagctttaccccgggctcggagtttgcaggagagagaagtggggagcaagaagtgaacctcaggggctcacagggttcccgcagatgctcaggccggccaggaatgcatctctggctctctgttcccacggacgtcactgcctcagccagcctcccccagagcccgccagccgctaagccggggccacacctgggggtgatttcatgcctcacctccagtaggcaccttggtttctttgggctaatctctggctcccttgcgctaactcttgctctcacccagctaatccctgcctcaccctgactgccccaggggctgaccactaacaaccaacctggccctgtytgggggttccaggctcctggcctggccctgaccagttcttaattaacctttccttcaccttgactaactcctgccttcctggtctgttcctttcagcagaaactaatggtttgtggatttttttctgactaacaacaggtctaacattcctcgttactgttaacagcttggatgtcggcatggctgggaaggggctaacacagctttgaacttggctaacacaggtttgaacttggctaacacaggtttgaacttgactaacacagggaaaagcatagctaacaattttgggcgtggtggctgctctgagtcagaacaatcagaagtcggtaaagatggtagttttctaaaggaggtgccagggctctggtgtggaccaagcctgatggagcagtggtacccaccaaggtggggtcagaagtatagccagtct

SEQ ID NO: 122 - DNA, 1579 bp, Homo sapiens
cccgtgtcatgagggatggtcatcatcttgtgtgatccttggagatggcaggaagccctggacatacatggtgtgggggctcctccagaggctgttgggatcctcctggatgtggtgtgggcatggaaggaaggccagtggagacaatggatgatcttgttcttagcagatcactggatgtggcagggagtcctaggacatgtgtggtgtgggcttcttcaggtgctgcacactcgtatttccgctgcacttcccaggtggtgttggcatgaggaaaggaggtatcttcgagggacaatcttcttcttgtgcgatccttggagatgccatgaggcccctggacacatgtggtgtgggctcctttggaggctgttgtatcccttctgaatgtggcgtgggcatagaaggaaggccagtggccacgagggacaatcttggtcttgggagatcctggaaatgatagggagtcccttgatatgtgtggcatgggctccttcaggtgctagcggattccttaggatgggacaaacactgtgcgtggatcgatgatgacttccatatatacattccttggaaagctgaacaaaatgagtgaaaactctataccgtcatcctcgtcgaactgaggtccagcacattactccaacaggggctagacagagagggccaacatcygttttttgacatgggttataccaaggcatccgttcaggcttaggatggggtcttttatgggtgatgggggtcacaggagagtggtggctcccatgtataggaaatttcttgtttgaaggactgtcagtgagggtgggtaacacatgcattgtctgcaggactaggtgaatgtccatgtggcctagcaagagttagctggtagcccgcctctggttgccaatttgttcttgagtccttgttctgagttcctggaaggaaacagatttgtctggttgggaggagaatacaaggccacatctttgtcgtttgttggctaactttgtccttggttgaggacattagagttttggtcaccaggcatagcctatgtgcctgtgtgcccgtgttgtatcccatgtgtttgggggacatgtacattgcatgaactagtgagctcctgctcattgcttctgatacccaaggagtccctggcttatcctaaacccaatataggttaaagcctttctcattaggggcccagggtcccaaggcttttgtgagtatcattgtaggtattgaagcaacgatgttgagaaggatgctgaacatgctctttagtgggatgacgtactctgaaggctcctgacccccagatgagcatccttgtgtccgttaacttctgtgtttatgaacaggtgaggccagagacaggcagacagcagatgtattgcagggagctggatgacatggcccttggaacctgtgcacatgcctgcctttctgatgcacgtccatgttttctctgcacctccccggtggtgttggtataaaaagcaggcttacatcagcaagggatgattgtcgtctcatgcgatcctgggagatggcagaagtcccgggacacatggagtgtgggctctttcgg

SEQ ID NO: 123 - DNA, 1595 bp, Homo sapiens
acctcagcacagaccctttatgggtgtcgggctcggggacggtcaggtctttctcatcccacgaggccacttttcagactatcacatggggagaaaccttggacaataaacggctttcaagggcagggctccctgcagctttccacagtgtatcgtgcccctggtttattgagactagagaatggcgatgacttttaccaagtatactgcttggaaacatcttgttaacaaggcatgtcctgcacagtcctagatcccttaaaccttgatttcctacaacacatgtttttgtgagcttcaggttgggtcaaagtggctggggcaaagctacacattaacaacatctcagcaaagcaattgttgaaagtacaggtctttttcaaaatggagtctcttatgtctttcctttctacatagacacagtaacagtctgatcgctctttcttttgcctacactcactgaactgcccttcccctttgctgggccatgaccacggggaacaggtccactgtcctccctgcgtggtgcacgatggatgctcagactccatcctcaaggctggcaagaagacacgttgagacatgtgcctcctgatacaggtgatggctgtggagcccacaggactggaacctcacactgcagggctggaggcacagaccatttactgttctgtgccctggggggctcaaggcacagagctcctcattagccaaagtcacccaagttccccaacctcttaaagatttcctcatcatcatgcaagaagaagagaaaagtgagtgtccatagaagctttggggctcttcctctaatcaggagaaagctggtgtgtattcttcrcttctttctttkctttttaaasatccaactgctttaattttcatcttttattrtgggaaaatataccaygtataaatattaaaaattataaatatatattagtkcatatagaatggccagtataaacatttacartttccactstttttcagtttacagtttmatgacattaartaygttcacattgtttagcaaccatcaccgycatcrtctccggaacagttttatytttcaaaatggaaattgcamccattcrccaagctctccactcctctctctygccyacccctgggggccacctttctagtttgcaactctakgagtytaactactctagacacttgatagataagtggaatcataccgtgtttaatttttttttttagaggtagaatctttctctgtcacccaggctggagtgcagtggcgtgatctcggctcactgcaacttccacttcgggggctcaagcaattcttatgtctcagtctcccgagtagctgggattacaggcgtgcgctatcatgcccagctaatttttgtatttttaatagagacgagctttcaccatattggccaggctggtctcgaactcctgagcttaagggatccacctgtctcagcctcccaaaatgctggggttacaggtgtgagccactgagcctgggcatgtttatccttttgggatttatttatttcactgacgataatgtcttcaagggtcatccatgttgcggcctgcatcaaaagtgcctg

SEQ ID NO: 124 - DNA, 1459 bp, Homo sapiens
cgggagtctaacacgtgcgcgagtcgggggctcgcacgaaagccgccgtggcgcaatgaaggtgaaggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgccggccctgcgggccgccggtgaaataccactactctgatcgttttttcactgacccggtgaggcgggggggcgagccccgaggggctctcgcttctggcgccaagcgcccggccgcgcgccggccgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcaccaagcgttggattgttcacccactaatagggaacgtgagctgggtttagaccgtcgtgagacaggttagttttaccctactgatgatgtgttgttgccatggtaatcctgctcagtacgagaggaaccgcaggttcagacatttggtgtatgtgcttggctgaggagccaatggggcgaagctaccatctgtgggattatgactgaacgcctctaagtcagaatcccgcccaggcggaacgatacggcagcgccgcggagcctcggttggcctcggatagccggtcccccgcctgtccccgccggcgggccgcccccccctccacgcgccccgcgcgcgcgggagggcgcgtgccccgccgcgcgccgggaccggggtccggtgcggagtgcccttcgtcctgggaaacggggcgcggccggaaaggcggccgccccctcgcccgtcacgcaccgcacgttcgtggggaacctggcgctaaaccattcgtag

SEQ ID NO: 125 - DNA, 2071 bp, Homo sapiens
cgcgtccgattaaattacatacttagtaaatagatattaattattttttgaaactcttgttagtgggaagaatatggtaaattttttgttaaataaaatagacccttatgtttagcattttgtttttagagaactattctggtactatcagaacaaatacataaaataacttcccatagagaacaggatatagcaataatagctccttagatactcagtggcttctgactccaatcaaggtcttgttgatattatatagtaaaaataaaaccaaaaataaatattattcaagtggctcttctaagcatgtgaatcatgaagcactgaaatatgtattttaatgatgatcttatttattcccatttttgcccttagttaacatttactggtgctcacctaggattggctattctgagggattgcatagaaaccaagctccacttgctgtccttgggaaggttataactgaatgcagctctttatttrgactaaagtgtcaggatatgcattagattctctcctgaaccaaaaacacaacagtcattatctgtgaaccataatttaaaaatctttctagaataacaacagcagactccactcttgtttgtctaaaagagccctactgggtatggatcattctgatgacagatttatacaaaatgattcaaaccagtaacttagtaaaattgaccttcgcaaaacctcactgggggagtgccttgtagagctgtgggtgggactgcacattcttctcctcttagtaaaagataggcccactttattccaagaataacacttagcacataaactcttcttccagctcgttagcagcattagcaccttctgaattccaccctctcagaagaatccacagtgtttgaacaatttgcataaaggtcagctagcatcctgctgccaagccactgcatagcatttgtgataagaaggaccaactctaggctcaatatgaagggatttagttctgtaagcagcaaaaaagcttctttatcaagtcatcttacctctaattcttttccagtrtgccaactccaaagtcaacattaaaaatgtaaatggacctgtgtaaatatcacagagagcttttccttatacatctcaatgctgagagttaaaatattcccaggttaaaatttttttaaagtaccaataatagagctaaatacaatgacatttgcttttaaaaggtggatattttatttctgctttttgaaaatacttatttagtattgacttggaagccaatttggtcctttaataagtaaagaaaataatatgtttaaaaatgtaaatgktttacaaatttgaaactttcataattgtattaatcagaaaacaagcacattgccattctttgaaactcatgtttctagacatgacagcagtaataaaaggatgaaaacaagtgtcttcactaagcgtatggccaataaatgggacccaaacgttcaatctgttcagtttaccaaggttcagaaatacgtaatttagcaggaaactataaataccagtgctatcacagccacacatacacacacacagacataaaataaccaaacatctcatttctaggaaagagataacactaaaggcatcataggtttaactgaaatacgttatatgaagttttacaaaaaggtcaacagaaagctcatttgtgaaaacatactctcatgggagcttctttaacattagttcagaggttaatatatttcctggaggtgttttcctagaattgattgcactattgcatggtaataacatttaattgttaaggaaacattatatataggttcaaattatcccttaatgttgatttctccccttttccatggattttgatactaagaaacaaaatgctttgagattttggtaactattttgattttgataaaacatgttaaaatagaaggacatgatatttttctatagtttccatcaggaagagtacatcagaaacttctccataaggaaagaaaactgactctctcttgaactaggtgttgataaaatacactaatggctttcttaattttattttattaggagaaaa

SEQ ID NO: 126 - DNA, 477 bp, Homo sapiens
gggaggttacggccgaggcggcggcggcggcgagcccgggggcgaggcgcggacgggaacaggaaaagcctccggcagcccctgcgggcggcggcgcagccacggccgcgctccgaggtgaagccgcgcgcggagaggaagcgggtgttttcccctctgcctttcggcccccgcccttcctttcagtttctgcccgctcgctcggaagttggcggttgacaaaaatggcaggagccggggcccgggccggttgccgcagcgccgcggggaccttctgagttggcccggtggcagggagactcgtgcaggggcgtccgatgcgcggggcccggggcctcgggagagctcagctgctgcgggccccagacgaggcgacagggatggacttgcgtagacagccagcgccgggccgccgggcgcgcggtctgggagggcgtgccgccgcggcgccgggccgcgctctgtgaaccggcgagg

SEQ ID NO: 127 - DNA, 1446 bp, Homo sapiens
taatccccaggtccctgggaggggtgctcatgctttgggtgggggaagcaatggtgacaggtctggtgggcctgatctcagggcatcagggtgtgcagagctccaggaggtagtaggcagggcaggcagtctgtggtgttggttgtggagagcctgacctctgggctggtgctagagtgtggtgatcctgctgttgagtatgggtggggttgctatcagtggtcccctgcagggagctctcaggttctgaggggtgtacactttcaactctggcagtagcagtgtccacagtggtgtgtgtgaagagcctgcactcatgacatgcactagagcacagaggccatgcttttgaagggggcagggttgctattcagagccccaaacaggcacttctcagtttctgggtagtgtttgctttgtctcctggctgcagtcagtgactgctatcatgttcaaaggggtcagatggatcctgcctttctgggtgtgaactcaagcacagaggctgtgttgttggtgggaatggggttactatttgcatcctcagacaggcagctgtcaggctcactcactttggctccccgtggcagcagcactattgtgatatgcagaaaggggaagggatccattttcacatgagcccaagtactgagaacatactgctaatagggatgtggttactgtttacatacccagactctcagatttaaggtttgcttgctttggctttcagaggcagcagtggctgcarcartgtggagagttggggaagggatcttgacctctgtgcataagctagagcacaaaggccatgctgctagttagggcagggtggttccctgccctaatggtaccaggtaccattggtatcattataccaggcagggagctcttgggttctgccaagcacatgcactggttccctttgtctcaggagaagcctccttgatgtactgcgctatcatttccttgaggagttgtactccctgtgggttagagtgctggggaccccacaacaccatcgggtccagccaccattgtgccactgaagccctccaggtggatgccagggaattctactgggggttcacagggtgtgaagatgtggaattgttggttctcagaagaggatgcagtctggtggaagctggactctggccatagtgccctactgcagctgcttatgtcttgctatgtgatgtggtgcaagtttcccgcttgcagcaatgccctggcaggcctctagatcaccacgctgtagagtccccacctatgctaatctcagagctgtatagatggaagaggtctcctgtggttaggattgcagtagtctaaggtaagactgtgtacccctaacggctcacactgaccctttccctataatagggagccgttccaggatcccagctggtcctggctgagctagctgctagcttcctctccttc

SEQ ID NO: 128 - DNA, 472 bp, Homo sapiens
gagggcgcattcggccccggacgaaggtactcgcagcacttggagcgcagaaccggccgcgcccgatcctccgagcggcggcgacggctgttgctaagggaggggacgcgcgaggaagcgcgacccgggcggcagacggcacccagcgccaccagccgagcggcgccccctccccaggacccttaaccgcgccgcgtcccggtcgcgcccgccgccctttgaaggagaagcaagtgccgtccccacccccggaaggcgcccccaggagccggagcgacctcggagcgccactcggattttggatttcggtctcgcattccgcggccgggactttctcgaggaggacgcgcgctgctccgcgcccccgagtgcccggaggacccggcatccggggagcctctcgcccctgtcccggaggcgcggcgaggattggcggcgcccgccgcccccagccccccagcgcgcgccgggg

SEQ ID NO: 129 - DNA, 1102 bp, Homo sapiens
ttcggcacgagggtggggcccaagagggaagatgaagcgagagatgccsrgaccagtgggagacgccaggacttcggaagctcttctgcgccacggtgggtggtgagggcggctgggaaagtgagctccagggccccaggagcagcctgctcgtgggtgcggaaggaaaaaggcacaggggcttggtgtgggcggcttttggctgggagaagtttgcacgtagggagaatagtagccagtgtttgcagagcacttactatgcaggaaggcctgtcctaagtattgtaagtgtattacatcatgtacaagtgtctgtgattaaccccgtcttgcagagaaggaaacaaaagtacaaacagaaaatgtaactaagcatgcaattaataaaaagggaccaggttttgaacgcgagcaatctggctcaagaatctgcgcccaaccaccggctcctgttcttagagatgaacgtggagtcctggagactgctcaacattgtgacttgactgtgagcgtacgcgctccctgtccccaggagacagatttccagtgcaatcatagaaagtgcctgtgtgggcttcgggagatgtgtctgccttggggagaattttccttttcagctagagccaggcccaggatgttgacgtcagtgagacgctggtgacgttctctgctccagtggctgatgagaaaagttcctccaagccagctcagttgagaagaattaagttctctgggtcccactggcttcacctacagatgccaactttgaggccagtgaactgtgaggccagctgggctgattgccatggcaacaggaattggaccaaagtcaccggaggatggagagggaagacacagtggtggcttccccaggtcttggaccacaaggcacagccgtggcctccaggaaccctgagataacccgttagtgggtcctgcactccaacagagctcatgcaatcagcctctggtcctcaccctcctcccattggtggccgttgtgctctctaacattgacattgagcagtgagtgctccagatcttgttccactgattttttccactggtctccagtctagcactttctgaaattcatccaagc

SEQ ID NO: 130 - DNA, 1243 bp, Homo sapiens
gcgtccgacactggtgacatgttgctgtatgcttggatgagtacgctggtgacacgttgctgtattcttgggcgtgtacactggtgacatgttgctgtattcttgtgtgaatacgctggtgacatattgctgtattcttgggcgtgtacactggtgacatattgctatgttcttaggcaagtacattgttgacatgttgctgcattcttaggcaagtacgtgggtgatatattcctgtattcttggacaagtacactggtgacatgtagctgtattcagaggtgagtacactggtgatgtattgctgtattctagggtgagtacactgttgaaatgttgctgtattcttaggtgagtacactggtgacatattgctatattcttgttcttcgtgtctagcaactcatacatgtttaccagaatattcctaaaggttcattttcaccatcaattctacccaaaactcggttagcccttttaacaggcagattcagcttttcctttgtttcaggaaattttctttttttgtgcttaatcacggcctctcctccatctacctcttttcctccccctgaaactcctatgttatttgcacctgatgtcctgggtctgttttcaaatcttttctctcatgttttcaatttctttgtattcctgtcaattcaagatttttcttctacttaatctttgaggccattaatttgaatcttaatgatcaccttcaattcatttgcaaccgtttttcagtaggctttattttttggaacaatttctgcttcacagcaaaattaagcagaaagtgcaaagagctcccataaccacctgaccccacacatgcacagcctctcctactatcagcatgccacacctactatcaacatgccacaccagagcagtacattgcttacaatcaatgggcccgtgtggacacatcataatcaccccaagtccattgtccacattggagttaacattccgtgttgtacatttttttggattttgataagtataatgggaagaggacagacactgatcttcactgtgttctgtggctctttgtggtccaagtttttcttcagacccatcacattccaatcttctcccagaccatggtctccaatgctgttacccaagttctatcccacccagagtttcaagtgaagcctaaaaccttatccacaaccttacgacctctctgcccactgtgctgcagagcagaggctgaaatgggttggagtgaaag

SEQ ID NO: 131 - DNA, 764 bp, Homo sapiens
ggcagaggagaaggggaggagcgcgattgcgcccgggatgggttgccagaccagctggggcggtggtggtccagaggcccgaggtcggcgggacctgatcgaaggcagcgccgcgtcgaccaccccgggagccggacgcttgggagccccagcccggcagcggcgcccggtcactgaagttgcgccccaactcccagccgcctccaagcttctcgagctaagtttcctgacccctccaagggagtctcacagagctcggtggccctcggccttgccaacgtcactttaactgtttggaactcgtgagcaagaaccgagaagtggagagcccagccggggagttttcagcttttctgtttcacttcgggcttcttctattcaaatggctctgcgctggccaccgaatcctgaatgaggcggggctcctctgccccaactccagcagcgggaacttggttcccctgggcagccggggcagggggcgccaaggccgtggcgataatgaaggctgagacggccaaggccagcgggtcggcgcggggcactctcgggccggagtggccatcggccggagttcaggaggtctgtgacaagcagggaacaaggcaacggacggcgcarcccagccccggctgacggacgctggcgactcagacatggacagtagctgccacaacgcgactaccaaaatgttagcgactgctccagctcggggcaacatgatgagcacgtccaaacccttggctttctccattgaacgaatc

SEQ ID NO: 132 - DNA, 486 bp, Homo sapiens
ggaggcagagttcggggaaagcgtcggagttcgggagaccagggtccagcatgggtttcagcacagcagacggcgggggcggcccaggcgcccgggatctggaatctcttgatgcctgtatccagaggacgctctctgccttgtacccaccgtttgaagccacggcagccacggtgctctggcagctgttcagcgtggccgagaggtgccacggtggggacgggctgcactgcctcaccagcttcctcctcccagccaagagggccctgcagcacctgcagcaggaagcctgtgccaggtacaggggtctggtcttcctgcacccaggctggccgctgtgcgcccatgagaaggtggtggtgcagctggcgtccctgcacggagtcaggctccagcccggggacttctacctgcaggtcacgtcggcggggaagcagtcagctagactggtcttgaaatgcctgtcccggctgggaagaggcacag

SEQ ID NO: 133 - DNA, 1238 bp, Homo sapiens
ccccgcgtccgcacctggccaggtccaaagtattaaaggatggataggatgttaggtaaagatacaaagttcaatttgtggagatgcatagtaacttccacaggcatcaagtggaagagtgagaatgggtcgtaatgttagtttgttactcagcagatgccagctgttttaattatacataaacgctactggcagtaaagggagagcttgaacagatgtccacgtgaaactccagggagaggagcatgggagtcagagtcagttacctgacctcactgagcctgtttctcctgtgaaatgggtaatgaggctgcttactcacagtggtggcaagactcagagatggttaccacctgcacagcatttaggactctggagaagtgtttgtgagccattttggaggggtgaacctttgtccttcaagaggggctggatttttggcaggacctgaagaaccaaggatgaccgcacagtcacaagctgtctccctgggctcaaggtggctcccactgagggaaggggacggaggtatcagccagtgcatcaggacctggggtcgtcactcccaaggggccattaccctgttcagtctccgtggccactctgggggagggaggtaaacctttacaggtaaggcccagagtgaggcccagagacagagtcatttgtgagcacgccaggctgatgagcggcagggggaaaattcaaatctggggagggtctgaccccaaagtccaacatctctggagcctcctgcccatgtcaggtgtttggattaatgggatatcccagaaatagtgtgtgcagcctcccaggggacaacttctgctgtcagccacccagaccagtcagccgcggagagcagcagcctgcagatgggacaccagtgctgagtgggacaggtgctggcttggccttgggatgtcacatgcataccctcccagtggacgtgaggattccaggggctcatgggatctgcctgctgcacccacaggtgtggcaggcgtgcttgtgggacacccgtttgacacggtcaaggtgagtctcatcgctgcttttttttcctcggcgcgtacattggagagaggctcacagggttggggtggcttggaagcctgtttccgtgtacagccccaggtgggcagcttgcttttacaccaggccgggttgaaccttcctcactgctttgtcctggcatctcccagctggggctgatccacatgctgggttcatggcca

SEQ ID NO: 134 - DNA, 1205 bp, Homo sapiens
ttgcaaaattaaaaaaaaatctcaacagtacagcatgttctttatatattatctgaaagataattttcagaaaaaggtraaacaatgacttgcaccaagatattaaaatacacaactcttaaagattttattttacacatrtgatagaagggaactaggcagatgttagaaatagtttaaaggaaaagtgaaaacaatacaaatttatatggagtaaaggaattttgaaatgagttgcaaatggaaagaaaacttttttatttatttattttcaaattttttacaggagaaagaagccagtaaaaatcactactagacagggcagaagatagatagatagatagatagatagatagatagatcgatctatgtctatatatctccatcagttacctgcaatttgcaaagaattgtaaaatagttcaaagacaatgaacaacccagaagtatgtgttacagttttccattgaaatacattttttaaacatatctaataggtatgtcttaactagcgaattcacaccactcttcagtgagaggactatttattgatcatctgcctgtgtgttgcaggttgctgtctacctttttcaaatttgaagcaaagattttcattaaaagattttcactagaattaattaaaaatcaaagcccaaatcaaaacagaatacacagcaagctgtgctagtgacatggatgacaacttctcctggggattacaactctcagggtgacatccgtgtagatgattctgtaactgttaaaatgaaaaactcccaccctgtgggaacagagccgggtgagccctggcttccacacagtgccaccctgagaaggcgaggkctccccagcgtctgtctgcagtgcagccagggcrgaggaatgaagtgtcacagcaggaagcagatggctgcatttgcagataatcaatctagagacttgcagccctgagtttcaggggaacttgtctaagtagcatcctgtcgctggaaggcatctaatgaactaagttactggtgttcttgcttgtcagatagccctggaacactgtctggattttataatcattttcttgagattgacaaagtctaaattcttgctgatcattgacgagtctaagttgtaaagaatgctacccatggatggaactttttgcttaaacttaagaaagggaggagaaataacagcagcggtgccccgtgaag

SEQ ID NO: 135 - DNA, 1414 bp, Homo sapiens
cgcgtccgctgggagctcaggaaggaaggagcgcccagaagcagggacagggagctggttggggaggaccagaaatcaggttatcaatactctggctgaccatcatcatcgtgggactgactttggtggaagtccttggttacatgtcattattgcgtttccgacaagttataaagttgtcattaccctctggatagtttacctttgggtgtctctcctgaagactatcttctggtctcgaaatggacatgatggatccacggatgtacagcagagagcctggaggtccaaccgccgtagacaggaagggctgaggtccatttgtatgcacacaaagaaaagagtttcttcctttcgaggaaataaaattggcctgaaagacgtcattactctacggagacatgtggaaacaaaagttagagctaaaatccgtaagaggaaggtgacaacgaaaatcaaccatcatgacaaaatcaatggaaagaggaagaccgccagaaaacagaaaatgtttcaacgtgcgcaagagttgcggcggcgrgcagaggactaccacaaatgcaaaatccccccttctgcaagaaaggctctttgcaactgggtcagaatggcggcagcggagcatcgtcattcttcaggattgccctactggccctacctcacagctgaaactttaaaaaacaggatgggccaccagccacctcctccaactcaacaacattctataactgataactccctgagcctcaagacacctcccgagtgtctgctcactccccttccaccctcagcggatgataatctcaagacacctcccgagtgtgtgctcactccccttccaccctcagcggatgataatctcaagacacctcccgagtgtgtgctcactccccttccaccctcagcggatgataatctcaagacacctcctgagtgtctgctcactccccttccaccctcagcggatgataatctcaagacacctcccgagtgtctactcactccccttccaccctcagctctaccctcagctccaccctcagcggatgataatctcaagacacgtgccgagtgtctgctccatccccttccaccctcagcggatgataatctcaagacaccttccgagcgtcagctcactccccttccaccctcagctccaccctcagcagatgataatatcaagacacctgccgagcgtctgcgggggccgcttccaccctcagcggatgataatctcaagacaccttccgagcgtcagctcactccccttccaccctcagctccaccctcagcagatgataatatcaagacacctgccgagcgtctgcgggggccgcttccaccctcagcggatgataatctcaagacaccttccgagcgtcagctcactccccttccaccctcagctcca

SEQ ID NO: 136 - DNA, 1218 bp, Homo sapiens
gagacggagtctcgctctgtcacccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgccctcggcctcccaaagtgctgggattacaggcgtgagccactgcgcccggccacatttcacttcttaagtcttctgtgtttttgggtatcaaatattcccggagagatgctcttgaggatctaagatccagctgtgggatgaggtgtacttcccaccctgccacaatcactgggcctgcccagacgggcagaggccctgtgcgccccacctgcctctctcacgtggactctgggggtcagagctgggtggggtgtgccgcgtgtgggtcctgagtggccagggcagggtcagcagcacaggaagctgcccagggggtccttgcaagcgtgggctctggccagcgtctgggggaggctgtgctaggcggggcctcccgtgggcatgtccctggagctcacaggctggcgccctatgcccatctccagatagcctgggctggaagctcttctacgtcacaggctgcctgtttgtggctgtgcaraacttggaggactgggaggtaaggccggctcgggtgcgggacagagtccagggctgttcagctcctgggttttttgcaatgggaatgaaaggaggaggaagggccctgggtggcctagcgcctccccgtcctgaagcgttggtccctgcttggaggtctccgttcatcaggacatggcccctgcactcatctgggaccgttcttggccaaggaattccccgaaggcatttttctcttagaagctctccatgactatcttcaccaaagtgctttcttcccagagttgccacaatgggatgcgagtcagctttccccgtggccggccctcccacctcggagcccctcatgagtcctttcagcctggcccagtgctgccctctgacctccatgccctcgtttgctggttccactgcctccctgcacttgttttgcctgcaggggtggagcaagcgcctgctgcacctgcccacctctccatttcccaacaggagtcgggttggctgccg

SEQ ID NO: 137 - DNA, 2588 bp, Homo sapiens
ggaagaatgttaaccccagaggcaacaaaagaaattaaattagtggaagaaaaaattcagtcagcgcaaataaatagaatagatcccttagccccactccarcttttgatttttgccactgcacattctccaacaggcatcattattcaaaatactgatcttgtggagtggtcattccttcctcacagtacagttaagacttttacaytgtacttggatcaaatrgctacattaatyggtcagacaagattacgaataataaaattatgtggaaatgaccmagacaaaatagttgtccctttaaccaaggaacaagttagacaagcctttatcaattctggtgcatggcagattggtcttgctaattttgtgggaattattgataatcattacccaaaaacaaagatcttccagttcttaaaattgactacttggattctacctaaaatwaccagacgtgaacctttagaaaatgctctaacagtatttactgatggttccagcaatggaaaagcagcttacacagggccgaaagaacgagtaatcaaaactccatatcaatcggctcaaagagcagagttggttgcagtcattacagtgttacaagattttgaccaacctatcaatattatatcagattctgcctatgtagtacaggctacaagggatgttgagacrgctctaattaaatatagcatggatgatcagttaaaccagctattcaatttattacaacaaactgtaagaaaaagaaatttcccattttatattactcatattcragcacacactaatttaccagggcctttgactaaagcaaatgaacaagctgacttactggtatcatctgcactcataaaagcacaagaacttcatgctttgactcatgtaaatgcagcaggattaaaaaacaaatttgatgtcacatggaaacaggcaaaagatattgtacaacattgcacccagtgtcaagtcttacacctgcccactcaagaggcaggagttaatcccagaggtctgtgtcctaatgcattatggcaaatggatgtcacgcatgtaccttcatttggaagattatcatatgttcatgtaacagttgatacttattcacatttcatatgggcaacttgccaaacaggagaaagtacttcccatgttaaaaaacatttattgtcttgttttgctgtaatgggagttccagaaaaaatcaaaactgacaatggaccaggatattgtagtaaagctttccaaaaattcttaagtcagtggaaaatttcacatacaacaggaattccttataattcccaaggacaggccatagttgaaagaactaatagaacactcaaaactcaattagttaaacaaaaagaagggggagacagtaaggagtgtaccactcctcagatgcaacttaatctagcactctatactttaaattttttaaacatttatagaaatcagactactacttctgcagaacaacatcttactggtaaaaagaacagcccacatgaaggaaaactaatttggtggaaagataataaaaataagacatgggaaatagggaaggtgataacgtgggggagaggttttgcttgtgtttcaccaggagaaaatcagcttcctgtttggatacccactagacatttgaagttctacaatgaacccatcggagatgcaaagaaaagggcctccgcggagatggtaacaccagtcacatggatggataatcctatagaagtatatgttaatgatagcgaatgggtacctggccccacagatgatcgctgccctgccaaacctgaggaagaagggatgatgataaatatttccattgggtatcgttatcctcctatttgcttagggacagcaccaggatgtttaatgcctgcagtccaaaattggttggtagaagtacctattgtcagtcccatcagtagattcacttatcacatggtaagcgggatgtcactcaggccacgggtaaattatttacaagactttycttatcaaagatcattaaaatttagacctaaagggaaaccttgccccaaggaaattcccaaagaatcaaaaaatacagaagttttagtttgggaagaatgtgtggccaatagtgcggtgatattacaaaacaatgaattcggaactattatagattgggcacctcgaggtcaattctaccacaattgctcaggacaaactcagtcrtgtccaagtgcacaagtgagtccagctgttgatagcgacttaacagaaagtttagacaaacataagcataaaaaattgcagtctttstacccttgggaatggggagaaaaaggaatctctaccccaagaccaaaaatartaagtcctgtttctggtcctgaacatccagaattatggaggcttaytgtggcctcacaccacattagaatttggtctggaaatcaaacttcagaaacaagagatcgtaagccattttatactatcgacctaaattccagtctaacggttcctttacagagttgcgtaaagcccccttatatgctagttgtaggaaatatagttattaaaccagactcccaaactataacctgtgaaaa

SEQ ID NO: 138 - DNA, 1863 bp, Homo sapiens
cccacgcgtccgtggtctcttcacatggacgtgcatgaaatttggtgccgtgactcagattgggggacctcccttcggagatcaatcccctgtcctcctgctctttgctccgtgagaaagatccacctacgacctcaggtcctcagaccgaccagcccaagaaacatctcaccaatttcaaatccagactccactggaaatcggactgttcaactcacctggcagccactcccagagcccctggaactctggcccaaggctctctgactgactccttcttggcttagcggctgaagactgatgctgcctgatcgcctcggaagccccgtagaccatcacggatgccgagctttaggtaactctcacagcggaaggtatacgcccagatggcctgaactaactgaagaatcacaaaagaagtgaaaatgccctgccccaccttaactgatgacattccaccacaaaagaagtgtaaatggccggtccttgccttaagtgatgacattaccttgtgaaagtccttttcctggctcatcctggctcaaaaagcacccccactgagcaccttgcgacccccmctcctrcycgccagagaacaaaccccctttgactgtaattttcctttacctamccaaatcctataaaacggccyyacccttatctcccttcgctgactctcttttcggactyagcccgcctgcacccaggtgaaataaacagcctcgttgctcacacaaagcctgtttggtggtctcttcacacggacgcgcatgaaatttggtgccgtgactcggatcgggggacctcccttgggagatcaatcccctgtcctcctgctctttgctccgtgagaaagatccacctacgacctcaggtcctcagaccaaccagcccaagaaacatctcaccaatttcaaatccggaacttgctacacatgccggaaatctggccactgggccaaggaacgcccgcagcccgggattcctcctaagccgcgtcccatctgtgtgggaccccactgaaaatcggactgttcaactcacctggcagccactcccagagctcctggaactctggcccaaggttctctgactgactccttcttggcttactggctgaagactgacgctgcctgatcgcctcagaagccccgcagaccatcatggacgccgagctttagcccgcctgcacccaggtgaaataaacagccttgttgctcacacaaagcctgtttggtggtctcttcacacagacgcgcatgaaagggaagacatacaaaaacaaggcctctgaggtaggtactactgagacagccaggtgggaaggactccttggcaaaactccaaccagccwgtgcacattcctcccagtgtacaggctggttggaatgtgcactgggatggagccatataagtttgtgtcgtttgcagtggggaggagcctggtccctcctcttcctgtgaggaacctggaattcaatctgtgaggttgttctggagatgttctggggagactgcattaaacacagcttcgcaccattgaataaactcagcaacaagccaatgcataaaagtaatctatgcttcaggtcacagaagcttcaaggggaaaaaaacagaatactctagggccattgttcacaaactcatctgaaaacatcctggaaaaattttcccaaacacatggaaagaaagagaggaaaaaagaagatatctgaataatgtggactagaataaagagctgccaggagctgtttatttaaaaacagtactttcttctctggctgagtccctggtattctctgctgcaatctgtagctgtagaattttgaaaaatgcaattaaattcaaatggtttgatga

SEQ ID NO: 139 - DNA, 717 bp, Homo sapiens
tcgacccacgcgtccgggcggccgggagggacgcggagccacagcccgacgcacggacggagggacgccggagcccgcctgaccatgtggaagctgggccggggccgagtgctgctggacgagccccccgaggaggaggacggcctgcgtggggggccgccaccggccgccgccgccgccgcccaggcgcaggttcagggagcaagtttccgaggttggaaagaagtgacttcactgtttaacaaagatgatgagcagcatctcctggaaagatgtaaatctcccaagtccaaaggaactaacttacgattaaaagaagagttgaaggcagagaagaaatctggattttgggacaatttggttttaaaacagaatatacagtctaaaaaaccagatgaaattgaaggttgggagcctccaaaacttgctcttgaagacatatcggctgaccctgaggacaccgtgggtggccacccatcctggtcaggctgggaggatgacgccaagggctcgaccaagtacaccagcctggccagctctgccaacagctccaggtggagcctgcgcgcggcagggaggctggtgagcatccgacggcagagtaaaggccacctgacagatagcccggaggaggcggagtgaggggggctgtgtggcaagtgtgccccgacatggtggccttttatgagtataccatgtagttgttgagtcttttcc